(19) United States
(12) Patent Application Publication
(10) Pub. No.: US 2022/0168403 A1
(43) Pub. Date: Jun. 2, 2022

(54) SUBCUTANEOUS TELOMERASE INHIBITOR COMPOSITIONS AND METHODS FOR USING SAME

(71) Applicant: Geron Corporation, Foster City, CA (US)

(72) Inventors: Anil Kapur, Foster City, CA (US); Patrick Murphy, Foster City, CA (US)

(21) Appl. No.: 17/376,517

(22) Filed: Jul. 15, 2021

Related U.S. Application Data
(60) Provisional application No. 63/128,708, filed on Dec. 21, 2020.
(60) Provisional application No. 63/053,455, filed on Jul. 17, 2020.

(51) Int. Cl. A61K 38/47; C12N 9/24; C12N 15/09; A61K 31/7125

(52) U.S. Cl. CPC A61K 38/47; C12N 9/2402; C12Y 302/01036; A61K 31/7125; C12N 15/09

(57) ABSTRACT

Aspects of the disclosure include telomerase inhibitor compositions formulated for subcutaneous administration. Compositions according to certain embodiments include a hyaluronidase enzyme and a telomerase inhibitor having an oligonucleotide and a lipid moiety linked to the 5′ and/or 3′ end of the oligonucleotide. Methods for subcutaneously administering the telomerase inhibitor compositions, such as in the treatment of a neoplasm are also described. Kits having or not having a subcutaneous injector are also provided.

### CROSS-REFERENCE TO RELATED APPLICATIONS

This application claims the benefit of U.S. Provisional Application No. 63/053,455, filed Jul. 17, 2020, and U.S. Provisional Application No. 63/128,708, filed Dec. 21, 2020, the disclosures of which are incorporated herein by reference in their entirety.

### INTRODUCTION

Hematologic malignancies are forms of cancer that begin in the cells of blood-forming tissue, such as the bone marrow, or in the cells of the immune system. Examples of hematologic cancer are acute and chronic leukemias, lymphomas, multiple myeloma and myelodysplastic syndromes.

Myeloproliferative Neoplasms (MPNs) are a group of blood disorders that occur when the body makes too many white or red blood cells, or platelets. This overproduction of blood cells in the bone marrow can create problems for blood flow and lead to various symptoms. MPNs can arise from precursors of the myeloid lineages in the bone marrow. MPNs are characterized by myeloproliferation without dysplasia, bone marrow hypercellularity, and predisposition to thrombosis, hemorrhage, and bone marrow fibrosis. There are various types of chronic myeloproliferative disorders. Included in the MPN disease spectrum are Essential Thrombocythemia (ET), Polycythemia vera (PV), Chronic Myelogenous Leukemia (CIVIL), myelofibrosis (MF), chronic neutrophilic leukemia, chronic eosinophilic leukemia, and acute myelogenous leukemia (AML). A myelodysplastic syndrome (MDS) is a group of symptoms that includes cancer of the blood and bone marrow. Myelodysplastic syndromes (MDS) includes diseases such as refractory anemia, refractory anemia with excess blasts, refractory cytopenia with multilineage dysplasia, refractory cytopenia with unilineage dysplasia, and chronic myelomonocytic leukemia (CMML).

Imetelstat or imetelstat sodium is a telomerase inhibitor that binds with high affinity to the template region of the RNA component of telomerase. Studies have shown that imetelstat or imetelstat sodium inhibits telomerase activity and is effective against cell proliferation in a multitude of different cancer cell lines and human tumors, such as described in U.S. Pat. No. 7,494,982. Imetelstat or imetelstat sodium has been used in clinical trials of patients with hematologic malignancies, including myelodysplastic syndromes and myeloproliferative neoplasms, and solid tumors. A clinical trial of patients with lower risk myelodysplastic syndrome showed that imetelstat or imetelstat sodium administered intravenously was able to achieve durable transfusion independence in certain patients. In addition, a clinical trial of patients with myelofibrosis showed that imetelstat or imetelstat sodium administered intravenously was able to achieve improvement in overall survival and bone marrow fibrosis that correlated with reduction of mutation burden and cytogenetic abnormalities of malignant clones.

For subcutaneous administration, a large volume of a liquid composition of a telomerase inhibitor is administered in order to achieve an effective dose. The term subcutaneous refers to a hypodermal layer of the skin, such as the subcutis layer. The subcutaneous interstitial matrix is composed of fibrous proteins in a viscoelastic gels of glycosaminoglycans. Glycosaminoglycans in the subcutaneous tissue include glycoHyaluronan (HA), a non-sulfated repeating linear disaccharide. Delivery of a large volume of liquid into the subcutaneous tissue may be limited and/or painful. Hyaluronidase enzyme (e.g., soluble hyaluronidase glycoprotein) facilitates rapid depolymerization of hyaluronan in the extracellular space of the subcutis and reduces the viscosity of the interstitium, increasing hydraulic conductance and allowing for larger volumes of liquid to be administered into the subcutaneous tissue.

### SUMMARY

Aspects of the disclosure include telomerase inhibitor compositions formulated for subcutaneous administration. Compositions according to certain embodiments include a hyaluronidase enzyme and a telomerase inhibitor having an oligonucleotide and a lipid moiety linked to the 5′ and/or 3′ end of the oligonucleotide. Methods for subcutaneously administering the telomerase inhibitor compositions for the treatment of cancer and the alleviation of symptoms associated with cancer are described. Methods for subcutaneously administering the telomerase inhibitor compositions in the treatment of hematologic malignancies are also described. Methods for subcutaneously administering the telomerase inhibitor compositions in the treatment of myelodysplastic syndromes (MDS) including diseases such as, refractory anemia, refractory anemia with excess blasts, refractory cytopenia with multilineage dysplasia, refractory cytopenia with unilineage dysplasia, and chronic myelomonocytic leukemia (CMML) are also described. Methods for subcutaneously administering the telomerase inhibitor compositions in the treatment of myeloproliferative neoplasms, such as Essential Thrombocythemia (ET), Polycythemia vera (PV), ChronicMyelogenous Leukemia (CIVIL), myelofibrosis (MF), chronic neutrophilic leukemia, chronic eosinophilic leukemia, and acute myelogenous leukemia (AML) are also described. Methods for subcutaneously administering the telomerase inhibitor compositions in the treatment of lymphoid neoplasms are also described. Methods for subcutaneously administering the telomerase inhibitor compositions in the treatment of solid tumors are also described. A liquid or lyophilized unit dosage form comprising a hyaluronidase enzyme and a telomerase inhibitor are described. Kits having a combination of telomerase inhibitor and hyaluronidase enzyme are also provided. Kits with a combination of telomerase inhibitor and hyaluronidase enzyme and a subcutaneous injector are also provided.

In some embodiments, the hyaluronidase enzyme is a recombinant human hyaluronidase. In certain embodiments, the hyaluronidase enzyme is rHuPH20. In some embodiments, telomerase inhibitor subcutaneous compositions include one or more soluble hyaluronidase glycoproteins. In embodiments, the hyaluronidase enzyme is present in the composition in an amount of from 100 U to 50,000 U. In some instances, the hyaluronidase enzyme is present in the composition in an amount of from 100 U/mL to 50,000 U/mL. In some instances, the hyaluronidase enzyme is present in the composition in an amount of from 100 U/mL to 2,000 U/mL. In some embodiments, the hyaluronidase enzyme (e.g., soluble hyaluronidase glycoprotein) is present in an amount that facilitates subcutaneous administration of the subject telomerase inhibitor composition. In some instances, the hyaluronidase enzyme is present in an amount that facilitates rapid depolymerization of hyaluronan in the extracellular space of the subcutis and thereby reduces the viscosity of the interstitium, increasing hydraulic conductance and allowing for larger volumes of the telomerase inhibitor composition to be administered into the subcutaneous tissue.

In some embodiments, the hyaluronidase enzyme is a variant of a recombinant human hyaluronidase. In certain embodiments, the hyaluronidase enzyme is a variant of hyaluronidase PH20 having one or more amino acid residue additions, deletions or substitutions to the amino acid sequence of wild-type PH20, such as a variant hyaluronidase PH20 having one or more amino acid residue additions, deletions or substitutions to the amino acid sequence of mature wild-type PH20. In certain embodiments, the hyaluronidase enzyme is a fragment of wild-type hyaluronidase PH20. In certain embodiments, the hyaluronidase enzyme is a fragment of wild-type hyaluronidase PH20 having one or more amino acid residue additions, deletions or substitutions to the amino acid sequence of wild-type hyaluronidase PH20.

The subject compositions may include one or more pharmaceutically acceptable excipients. In some embodiments, the composition includes one or more saccharides. In some instances, the saccharides include one or more monosaccharides. The monosaccharides may be present in the composition in an amount of from 1 mM to 1000 mM, such as from 10 mM to 500 mM. In some instances, the saccharides include one or more polysaccharides. In some instances, the polysaccharides include sucrose. In other instances, the polysaccharides include trehalose. The polysaccharides may be present in the composition in an amount of from 1 mM to 1000 mM, such as from 10 mM to 500 mM. In certain instances, the polysaccharides are present in the composition in an amount of from 100 mM to 300 mM.

In some embodiments, the composition includes one or more amino acids. In certain embodiments, the composition includes an amount of methionine. In other embodiments, the composition includes an amount of histidine. In these embodiments, the amino acids may be present in the composition in an amount of from 1 mM to 200 mM, such as from 1 mM to 100 mM. In certain embodiments, the amino acids are present in the composition in an amount of from 1 mM to 50 mM.

Compositions may also include one or more buffers. In some instances, the buffers are present in the composition in an amount to maintain the composition at a predetermined pH. For example, the one or more buffers may be present in the composition to maintain the composition at a pH of from 3.0 to 9.0, such as a pH of from 5.5 to 7.5. In some embodiments, the buffer is present in the composition in an amount of from 1 mM to 250 mM, such as from 1 to 100 mM and including from 1 mM to 50 mM. In certain instances, the buffer is a histidine buffer. In certain instances, the composition comprises a methionine and polysorbate 80 or polysorbate 20.

In any of the embodiments herein, the telomerase inhibitor comprises an oligonucleotide. In some embodiments, the oligonucleotide is complementary to the RNA component of telomerase. In some embodiments, the oligonucleotide is 10-20 base pairs in length. In some embodiments, the oligonucleotide comprises the sequence TAGGGTTAGACAA. In some embodiments of any of the embodiments herein, the oligonucleotide comprises at least one N3′→P5′ thiophosphoramidate internucleoside linkage. In some embodiments of any of the embodiments herein, oligonucleotide comprises all N3′→P5′ thiophosphoramidate internucleoside linkages. The telomerase inhibitor according to certain embodiments is an oligonucleotide that includes a lipid moiety linked to the 5′ and/or 3′ end of the oligonucleotide. In certain embodiments, the lipid moiety of the telomerase inhibitor is linked to the 5′ and/or 3′ end of the oligonucleotide via a linker. In some instances, the linker is a glycerol or aminoglycerol linker. In certain instances, the lipid moiety of the telomerase inhibitor is a palmitoyl (C16) moiety. In some embodiments, the subject compositions include imetelstat or a pharmaceutically acceptable salt thereof. In certain embodiments, compositions include imetelstat sodium.

In some embodiments, the amount of telomerase inhibitor in the subcutaneous composition is from about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, about 450 to about 500 mg, about 500 mg to about 600 mg, about 600 mg to about 700 mg, about 700 mg to about 800 mg, about 800 mg to about 900 mg, about 900 mg to about 1000 mg, about 1100 mg to about 1200 mg, about 1200 mg to about 1300 mg, about 1300 mg to about 1400 mg, about 1400 mg to about 1500 mg, about 1500 mg to about 1600 mg, about 1600 mg to about 1700 mg, about 1700 mg to about 1800 mg, about 1800 mg to about 1900 mg, about 1900 mg to about 2000 mg, about 2000 mg to about 2100 mg, about 2100 mg to about 2200 mg, about 2200 mg to about 2300 mg, about 2300 mg to about 2400 mg, about 2400 mg to about 2500 mg.

In some embodiments, the amount of a telomerase inhibitor is in a unit dosage form having an amount in the range of from about 5 mg to about 1000 mg, 5 mg to about 500 mg, such as about 30 mg to about 300 mg or about 50 mg to about 200 mg. In some embodiments, the amount of a telomerase inhibitor is in a unit dosage form having an amount in the range of from about 500 mg to about 3000 mg, 750 mg to about 2500 mg, such as about 1000 mg to about 2000 mg or about 50 mg to about 200 mg. The unit dosage form may be liquid or lyophilized.

In some embodiments, the concentration of the telomerase inhibitor in the composition is dilute (about 0.1 mg/ml) or concentrated (about 300 mg/ml), including, for example, any of about 0.1 to about 300 mg/ml, of about 0.1 to about 200 mg/ml, about 0.1 to about 180 mg/ml, about 0.1 to about 160 mg/ml, about 0.1 to about 140 mg/ml, about 0.1 to about 120 mg/ml, about 0.1 to about 100 mg/ml, about 0.1 to about 80 mg/ml, about 0.1 to about 60 mg/ml, about 0.1 to about 40 mg/ml, about 0.1 to about 20 mg/ml, about 0.1 to about 10 mg/ml about 2 to about 40 mg/ml, about 4 to about 35 mg/ml, about 6 to about 30 mg/ml, about 8 to about 25 mg/ml, about 10 to about 20 mg/ml, about 12 to about 15 mg/ml, or any of about 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2 mg/ml, 2.1 mg/ml, 2.2 mg/ml, 2.3 mg/ml, 2.4 mg/ml, or 2.5 mg/ml. In some embodiments, the concentration of the telomerase inhibitor is at least about any of 0.1 mg/ml, 0.2 mg/ml, 0.3 mg/ml, 0.4 mg/ml, 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 33.3 mg/ml, 34 mg/ml, 35 mg/ml, 36 mg/ml, 37 mg/ml, 38 mg/ml, 39 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml, 200 mg/ml, 210 mg/ml, 220 mg/ml, 230 mg/ml, 240 mg/ml, 250 mg/ml, 260 mg/ml, 270 mg/ml, 280 mg/ml, 290 mg/ml, or 300 mg/ml. In certain embodiments, the composition is formulated to include the telomerase inhibitor, such as imetelstat or imetelstat sodium, at a dosage of from about 2.0 mg/kg to about 20.0 mg/kg, about 3.0 mg/kg to about 15.0 mg/kg, such as from about 4.0 mg/kg to about 10 mg/kg, from about 7.5 mg/kg to 9.4 mg/kg, from about 9 mg/kg to about 11 mg/kg, and from about 11 mg/kg to about 14 mg/kg. In certain embodiments, the composition is formulated to include the telomerase inhibitor, such as imetelstat or imetelstat sodium, at a dosage of 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9.0 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10.0 mg/kg, 10.5 mg/kg, 11.0 mg/kg, 11.5 mg/kg, 12.0 mg/kg, 12.5 mg/kg, 13.0 mg/kg, 13.5 mg/kg, 14.0 mg/kg, 14.5 mg/kg, 15.0 mg/kg, 15.5 mg/kg, 16.0 mg/kg, 16.5 mg/kg, 17.0 mg/kg, 17.0 mg/kg, 17.5 mg/kg, 18.0 mg/kg, 18.5 mg/kg, 19.0 mg/kg, 19.5 mg/kg, or 20.0 mg/kg.

Aspects of the disclosure also include methods for subcutaneously administering a telomerase inhibitor composition to a subject. In practicing methods according to certain embodiments, a composition having a telomerase inhibitor and a hyaluronidase enzyme is subcutaneously administered to the subject. In some embodiments, the composition is administered to the subject by subcutaneous injection. In other embodiments, the composition may be administered to the subject from an implanted device, such as a subcutaneously implanted catheter. In certain embodiments, the telomerase inhibitor composition is administered to the subject with a subcutaneous bolus injector configured to subcutaneously deliver a predetermined amount of the composition to the subject. In certain embodiments, the telomerase inhibitor and the hyaluronidase enzyme are administered simultaneously. For example, the telomerase inhibitor and the hyaluronidase enzyme can be mixed together prior to administration. In some instances, the telomerase inhibitor and the hyaluronidase enzyme are administered sequentially. For example, the hyaluronidase enzyme can be administered before the telomerase inhibitor. In some cases, the hyaluronidase enzyme is administered first followed by administration of the telomerase inhibitor. For instance, the hyaluronidase enzyme can be administered immediately followed by administration of the telomerase inhibitor.

In certain embodiments, the subject methods include treating a neoplasm. In some embodiments, the neoplasm may be a solid tumor cancer. Examples of cancers for treatment according to embodiments of the present disclosure may include but are not limited to, e.g., Adrenocortical Carcinoma, Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Carcinoid Tumor (e.g., Childhood, Gastrointestinal, etc.), Carcinoma of Unknown Primary, Cardiac (Heart) Tumors, Cervical Cancer, Colon Cancer, Colorectal Cancer, Craniopharyngioma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, etc.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hypopharyngeal Cancer, Intraocular Melanoma, Islet Cell Tumors (e.g., Pancreatic Neuroendocrine Tumors, etc.), Kidney Cancer (e.g., Renal Cell, Wilms Tumor, Childhood Kidney Tumors, etc.), Langerhans Cell Histiocytosis, Laryngeal Cancer, Lip and Oral Cavity Cancer, Liver Cancer (Primary), Lobular Carcinoma In Situ (LCIS), Lung Cancer (e.g., Non-Small Cell, Small Cell, etc.), Malignant Fibrous Histiocytoma of Bone and Osteosarcoma, Melanoma, Merkel Cell Carcinoma, Mesothelioma, Metastatic Squamous Neck Cancer with Occult Primary, Mouth Cancer, Multiple Endocrine Neoplasia Syndromes, Nasal Cavity and Paranasal Sinus Cancer, Nasopharyngeal Cancer, Neuroblastoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sézary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like. In certain embodiments, methods include treating a subject having a neoplasm as described in U.S. Pat. No. 7,494,982.

In some embodiments, the neoplasm may be a hematological neoplasm. In certain embodiments, the subject methods include treating a subject having a myeloproliferative neoplasm. In some instances, the myeloproliferative neoplasm is myelofibrosis (MF), such as primary myelofibrosis, or myelofibrosis following previous ET or PV (post-ETMF or post-PVMF). In other embodiments, the myeloproliferative neoplasm includes Essential Thrombocythemia (ET), Polycythemia vera (PV), Chronic Myelogenous Leukemia (CIVIL), chronic neutrophilic leukemia, chronic eosinophilic leukemia, and acute myelogenous leukemia (AML). In other instances, the hematologic neoplasm is myelodysplastic syndromes (MDS). In still other embodiments, the hematologic neoplasm is myelodysplastic syndromes (MDS) with isolated non-del (5q). Myelodysplastic syndromes (MDS) include diseases such as, refractory anemia, refractory anemia with excess blasts, refractory cytopenia with multilineage dysplasia, refractory cytopenia with unilineage dysplasia, and chronic myelomonocytic leukemia (CMML). Methods according certain embodiments also include diagnosing a subject has having a myeloproliferative neoplasm. In one example, methods include diagnosing the subject has having myelofibrosis (MF), such as primary myelofibrosis. In some embodiments, the subject has not previously been administered a telomerase inhibitor (e.g., is telomerase inhibitor naïve). In some embodiments, the subject is a subject with lower risk transfusion dependent MDS who is relapsed or refractory to an erythropoietin stimulating agent (ESA). In some embodiments, the subject has not received prior treatment with a hypomethylating agent (HMA). In some embodiments, the subject has not received prior treatment with lenalidomide. In some embodiments, the subject is a subject who is non-del(5q). In some embodiments, the subject is a subject who is relapsed or refractory to a Janus kinase (JAK) inhibitor. In some embodiments, the subject methods include treating a subject having a myeloproliferative neoplasm, such as described in U.S. Pat. No. 9,375,485 and International Patent Publication Nos. WO 2019/023667 and WO 2020/028261, the disclosures of which are herein incorporated by reference.

In certain embodiments, the subject methods include treating a subject having a lymphoid neoplasm. In some embodiments the lymphoid neoplasm (e.g., lymphoma) is a B-cell neoplasm. In some embodiments the lymphoid neoplasm (e.g., lymphoma) is a T-cell and/or putative NK-cell neoplasm.

In certain embodiments, a telomerase inhibitor and a hyaluronidase enzyme are subcutaneously administered to the subject. The telomerase inhibitor composition and the hyaluronidase enzyme may be subcutaneously administered to the subject one or more times per day, such as two times per day or more, such as three times per day or more, such as four times per day or more and including five times per day or more. In some embodiments, the composition is subcutaneously administered to the subject for 1 day or more, 2 days or more, 3 days or more, 4 days or more, 5 days or more, 6 days or more, 7 days or more, such as for 10 days or more, such as for 14 days or more, such as for 21 days or more. The dosing may be administered in cycles of administration of the telomerase inhibitor composition and the hyaluronidase enzyme. In some embodiments, the cycle is once every day. In some embodiments, the cycle is once every other day (i.e., once every 2 days). In some embodiments, the cycle is once every 3 days. In some embodiments, the cycle is once every 4 days. In some embodiments, the cycle is once every 5 days. In some embodiments, the cycle is once every 6 days. In some embodiments, the cycle is once every 7 days. In some embodiments, the cycle is once every 14 days, in some instances the cycle is once every 21 days, in other instances the cycle is once every 28 or more days. The cycles of administration of the telomerase inhibitor composition may be repeated for 1, 2, 3, 4, 5, 6, 7, 8 or more than 8 dosage cycles, for a total period of 6 months or 1 year or 2 years or 3 years or 4 years or 5 years or 6 years or 7 years or 8 years or 9 years or 10 years or more. For example, a cycle of administration can be performed, followed by no administration of the composition for 1 or more days, and then a subsequent cycle of administration can be performed. The time between the cycles of administration can be 1 or more days, 2 or more days, 3 or more days, 4 or more days, 5 or more days, 6 or more days, 7 or more days, 10 or more days, 14 or more days, 21 or more days, or 28 or more days.

Without wishing to be bound by theory, the present invention provides compositions, unit dosage forms, and kits comprising a telomerase inhibitor and a hyaluronidase enzyme both as defined herein with compatibility and stability for use in methods of treating a subject by subcutaneous administration having a neoplasm. The invention also provides for such compositions formulated for subcutaneous administration where the composition is safe and tolerable for the subject being treated and enabling an effective pharmacokinetic profile for the telomerase inhibitor when co-formulated with the hyaluronidase enzyme. The invention further provides dosage forms with appropriate liquid volumes containing sufficient doses of the telomerase inhibitor for subcutaneous administration.

### BRIEF DESCRIPTION OF THE DRAWINGS

FIG. 1 depicts plasma concentration time profiles of imetelstat sodium after subcutaneous injection and intravenous delivery in rats according to certain embodiments.

FIG. 2 depicts telomerase activity inhibition (%) vs. treatment concentration (04) for various sample formulations according to certain embodiments.

### SELECT DEFINITIONS

The term “nucleoside” refers to a moiety having the general structure:

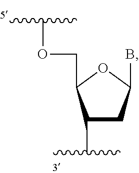

where B represents a nucleobase and the 2′ carbon can be substituted as described below. When incorporated into an oligomer or polymer, the 3′ carbon is further linked to an oxygen or nitrogen atom. Nucleosides may include 2′-deoxy and 2′-hydroxyl (i.e. deoxyribose and ribose) forms, and analogs thereof. In certain instances, a 5′-NH group can be substituted for the 5′-oxygen. “Analogs”, in reference to nucleosides, includes synthetic nucleosides having modified nucleobase moieties (see definition of “nucleobase” below) and/or modified sugar moieties, such as 2′-fluoro sugars, among other analogs. Such analogs are typically designed to affect binding properties, e.g., stability, specificity, or the like. The term nucleoside includes the natural nucleosides, including 2′-deoxy and 2′-hydroxyl forms, e.g., as described in Komberg and Baker, DNA Replication, 2nd Ed. (Freeman, San Francisco, 1992), and analogs. “Analogs”, in reference to nucleosides, includes synthetic nucleosides having modified nucleobase moieties (see definition of “nucleobase,” infra) and/or modified sugar moieties, e.g., described generally by Scheit, Nucleotide Analogs (John Wiley, New York, 1980). Such analogs include synthetic nucleosides designed to enhance binding properties, e.g., stability, specificity, or the like, such as disclosed by Uhlmann and Peyman, Chemical Reviews 90:543-584, 1990). An oligonucleotide containing such nucleosides, and which typically contains synthetic nuclease-resistant internucleoside linkages, may itself be referred to as an “analog”.

A “polynucleotide” or “oligonucleotide” refers to a ribose and/or deoxyribose nucleoside subunit polymer or oligomer having between about 2 and about 200 contiguous subunits. The nucleoside subunits can be joined by a variety of intersubunit linkages, including, but not limited to, phosphodiester, phosphotriester, methylphosphonate, P3′→N5′ phosphoramidate, N3′→P5′ phosphoramidate, N3→P5′ thiophosphoramidate, and phosphorothioate linkages. The term also includes such polymers or oligomers having modifications, such as to the sugar (e.g., 2′ substitutions), the base, and the 3′ and 5′ termini. In embodiments where the oligonucleotide moiety includes a plurality of intersubunit linkages, each linkage may be formed using the same chemistry, or a mixture of linkage chemistries may be used. When an oligonucleotide is represented by a sequence of letters, such as “ATGUCCTG,” it will be understood that the nucleotides are in 5′43′ order from left to right. Representation of the base sequence of the oligonucleotide in this manner does not imply the use of any particular type of internucleoside subunit in the oligonucleotide.

A “nucleobase” includes (i) native DNA and RNA nucleobases (uracil, thymine, adenine, guanine, and cytosine), (ii) modified nucleobases or nucleobase analogs (e.g., 5-methylcytosine, 5-bromouracil, or inosine) and (iii) nucleobase analogs. A nucleobase analog is a compound whose molecular structure mimics that of a typical DNA or RNA base.

The term “lipid” is used broadly herein to encompass substances that are soluble in organic solvents, but sparingly soluble, if at all, in water. The term lipid includes, but is not limited to, hydrocarbons, oils, fats (such as fatty acids and glycerides), sterols, steroids and derivative forms of these compounds. In some embodiments, lipids are fatty acids and their derivatives, hydrocarbons and their derivatives, and sterols, such as cholesterol. Fatty acids usually contain even numbers of carbon atoms in a straight chain (commonly 12-24 carbons) and may be saturated or unsaturated, and can contain, or be modified to contain, a variety of substituent groups. For simplicity, the term “fatty acid” also encompasses fatty acid derivatives, such as fatty or esters. In some embodiments, the term “lipid” also includes amphipathic compounds containing both lipid and hydrophilic moieties.

An “individual” or a “patient” or a “subject” can be a mammal, such as any common laboratory model organism. Mammals include, but are not limited to, humans and non-human primates, farm animals, sport animals, pets, mice, rats, and other rodents. In some embodiments, an individual or patient or subject is a human. In certain embodiments, the subject or patient has not previously received telomerase inhibitor therapy prior to certain embodiments, such patients are “telomerase inhibitor naïve”.

An “effective amount” or “therapeutically effective amount” or “clinically effective amount” refers to an amount of the telomerase inhibitor, administered to a mammalian subject, either as a single dose or as part of a series of doses, which is effective to produce a desired therapeutic effect.

As used herein, the term “neoplasm” or “neoplasia” or “neoplastic” refers to abnormal new cell growth. Unlike hyperplasia, neoplastic proliferation persists even in the absence of an original stimulus. “Neoplastic cells” refer to cells which exhibit relatively autonomous growth, so that they exhibit an aberrant growth phenotype characterized by a significant loss of control of cell proliferation. Neoplastic cells comprise cells which may be actively replicating or in a temporary non-replicative resting state (Gi or Go); similarly, neoplastic cells may comprise cells which have a well-differentiated phenotype, a poorly-differentiated phenotype, or a mixture of both type of cells. Thus, not all neoplastic cells are necessarily replicating cells at a given timepoint. “Neoplastic cells” encompass such cells in benign neoplasms and cells in malignant neoplasms. “Neoplastic progenitor cells” refers to cells of a cellular composition that possess the ability to become neoplastic.

A “proliferative disorder” is any cellular disorder in which the cells proliferate more rapidly than normal tissue growth. Thus a “proliferating cell” is a cell that is proliferating more rapidly than normal cells. The proliferative disorder includes, but is not limited to, neoplasms. A “neoplasm” is an abnormal tissue growth, generally forming a distinct mass that grows by cellular proliferation more rapidly than normal tissue growth. Neoplasms show partial or total lack of structural organization and functional coordination with normal tissue. These can be broadly classified into three major types. Malignant neoplasms arising from epithelial structures are called carcinomas, malignant neoplasms that originate from connective tissues such as muscle, cartilage, fat or bone are called sarcomas and malignant tumors affecting hematopoetic structures (structures pertaining to the formation of blood cells) including components of the immune system, are called leukemias and lymphomas. A tumor is the neoplastic growth of the disease cancer. As used herein, a neoplasm, also referred to as a “tumor”, is intended to encompass hematopoietic neoplasms as well as solid neoplasms. Other proliferative disorders include, but are not limited to, neurofibromatosis.

As used herein, the singular form “a”, “an”, and “the” includes plural references unless indicated otherwise.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present invention and are disclosed herein just as if each and every such sub-combination was individually and explicitly disclosed herein.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, methods and materials of interest are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

The compounds described herein can contain one or more chiral centers and/or double bonds and therefore, can exist as stereoisomers, such as double-bond isomers (i.e., geometric isomers), enantiomers or diastereomers. Accordingly, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

### DETAILED DESCRIPTION

### Subcutaneous Telomerase Inhibitor Compositions

Aspects of the disclosure include telomerase inhibitor compositions formulated for subcutaneous administration. The term “subcutaneous” is used herein in its conventional sense to refer to a hypodermal layer of the skin, such as the subcutis layer. The subcutaneous interstitial matrix is composed of fibrous proteins in a viscoelastic gel of glycosaminoglycans. Glycosaminoglycans in the subcutaneous tissue include glycoHyaluronan (HA), a non-sulfated repeating linear disaccharide.

Compositions of the present disclosure include a hyaluronidase enzyme. In some embodiments, the hyaluronidase enzyme is a mammalian-type hyaluronidase, such as an endo-beta-N-acetylhexosaminidase with tetrasaccharides and hexasaccharides as the major end products. In some instances, the mammalian hyaluronidase have both hydrolytic and transglycosidase activities, and can degrade hyaluronan and chondroitin sulfates. In other embodiments, the hyaluronidase enzyme is a bacterial hyaluronidase, such as an endo-beta-N-acetylhexosaminidase, that generates disaccharide end products by beta elimination. In still other embodiments, the hyaluronidase enzyme is an endo-beta-glucuronidase that generates tetrasaccharide and hexasaccharide end products through hydrolysis of a β-1-3 linkage. In some embodiments, hyaluronidase enzymes in the subject telomerase inhibitor compositions include a mammalian hyaluronidase having a neutral active site or an acidic active site. In certain embodiments, compositions of interest include a recombinant human hyaluronidase enzyme. In certain instances, the recombinant human hyaluronidase enzyme is a PH20 recombinant human hyaluronidase enzyme (rHuPH20). In some embodiments, the telomerase inhibitor subcutaneous composition includes one or more soluble hyaluronidase glycoproteins (sHASEGPs). In some embodiments, the hyaluronidase enzyme (e.g., soluble hyaluronidase glycoprotein) facilitates subcutaneous administration of the subject composition. In some instances, the hyaluronidase enzyme is present in an amount for rapidly depolymerizing hyaluronan in the extracellular space and reduces the viscosity of the interstitium, increasing hydraulic conductance and allowing for larger volumes to be administered into the subcutaneous tissue. In certain embodiments, the increased hydraulic conductance induced by hyaluronidase enzyme through reduced interstitial viscosity allows for greater dispersion, increasing the systemic bioavailability of subcutaneously administered telomerase inhibitors described herein.

In certain embodiments, compositions include one or more hyaluronidase enzymes (e.g., soluble hyaluronidase glycoproteins), such those described in International Patent Publication Nos. WO2004/078140 and WO 2006/091871 and U.S. Pat. No. 7,767,429, the disclosures of which are incorporated herein by reference.

In some embodiments, the hyaluronidase enzyme is a variant or fragment of a recombinant human hyaluronidase which is active and can degrade hyaluronan. The sequence (SEQ ID NO: 1) of wild-type human PH20 hyaluronidase enzyme is show in Table 1 below.

TABLE 1
Amino acid sequence of precursor, including
the signal sequence of wild type human
PH20 hyaluronidase enzyme (SEQ ID NO: 1)

MGVLKFKHIFFRSFVKSSGVSQIVFTFLLIPCCLTLNFRAPPVIPNVPFL
WAWNAPSEFCLGKFDEFLDMSLFSFIGSPRINATGQGVTIFYVDRLGYYP
YIDSITGVTVNGGIPQKISLQDHLDKAKKDITFYMPVDNLGMAVIDWEEW
RPTWARNWKPKDVYKNRSIELVQQQNVQLSLTSATEKAKQEFEKAGKDFL
VETIKLGKLLRPNHLWGYYLFPDCYNHHYKKPGYNGSCFNVEIKRNDDLS
WLWNESTALYPSIYLNTQOSPVAATLYVRKRVREAIRVSKIPDAKSPLPV
FAYTRIVFTDQVLKFLSQDELVYTFGETVALGASGIVIWGTLSIMRSMKS
CLLLDNYMETILNPYIINVTLAAKMCSQVLCQEQGVCIRKNWNSSDYLKL
NPDNFAIQLEKGGKFTVRGKPTLEDLEQFSEKFYCSCYSTLSCKEKADVK
DTDAVDVCIADGVCIDAFLKPPMETEEPQIFYNASPSTLSATMFIVSILF
LIISSVASL

In certain embodiments, the hyaluronidase is a soluble hyaluronidase. Soluble hyaluronidases include any, that, upon expression and secretion from a cell, exist in soluble form. Such soluble hyaluronidases include, but are not limited to, non-human soluble hyaluronidases, bacterial soluble hyaluronidases, bovine PH20, ovine PH20, and variants thereof. Included among the soluble hyaluronidases are human PH20 polypeptides that have been modified to be soluble. For example, hyaluronidases, such as human PH20, that contain a glycophosphatidylinositol (GPI) anchor can be made soluble by truncation of and removal of all or a portion of the GPI anchor. In one example, the human hyaluronidase PH20, which is normally membrane anchored via a GPI anchor, is made soluble by truncation of and removal of all or a portion of the GPI anchor at the C-terminus.

Soluble hyaluronidases also include neutral active hyaluronidases, such as the soluble human PH20 polypeptides. In a particular example, the hyaluronidase for use in the compositions, combinations and methods herein is a soluble neutral active hyaluronidase. Exemplary of hyaluronidases include a soluble form of a PH20 from any species, such as a soluble form of a PH20. Soluble forms of PH20 are known in the art, These include ovine and bovine PH20 polypeptides, and soluble forms of the human PH20 of SEQ ID NO:1. Soluble forms of the human PH20 of SEQ ID NO:1. Such soluble forms include truncated forms thereof lacking all or a portion of the C-terminal GPI anchor, so long as the hyaluronidase is soluble (secreted upon expression) and retains hyaluronidase activity. Such forms also typically are mature forms that, when expressed in a cell, lack the signal peptide. Full length mature human PH20 (residues 36-509 of SEQ ID NO:1) occurs as a GPI-anchored polypeptide. As known in the art, it is rendered soluble by truncation at the C-terminus. Such truncation can remove all of the GPI anchor attachment sequence, or can remove only some of the GPI anchor attachment. The resulting polypeptide, however, is soluble. In instances where the soluble hyaluronidase retains a portion of the GPI anchor attachment signal sequence, 1, 2, 3, 4, 5, 6, 7 or more amino acid residues in the GPI anchor attachment signal sequence can be retained, provided the polypeptide is soluble. Polypeptides containing one or more amino acids of the GPI anchor are termed extended soluble hyaluronidases. One of skill in the art can determine whether a polypeptide is GPI-anchored using methods well known in the art. Such methods include, but are not limited to, using known algorithms to predict the presence and location of the GPI anchor attachment signal sequence and w-site, and performing solubility analyses before and after digestion with phosphatidylinositol-specific phospholipase C (PI-PLC) or D (PI-PLD).

Exemplary of a soluble hyaluronidase is soluble human PH20. Soluble forms of recombinant human PH20 have been produced and can be used in the compositions, combinations and methods described herein. The description of and production of such soluble forms of PH20 is described, for example, in U.S. Pat. Nos. 7,767,429, 8,202,517, 8,431,380, 8,431,124, 8,450,470, 8,765,685, 8,772,246, 7,871,607, 7,846,431, 7,829,081, 8,105,586, 8,187,855, 8,257,699, 8,580,252, 9,677,061, and 9,677,062, which are incorporated by reference herein.

Recombinant soluble forms of human PH20 have been generated and can be used in the compositions, combinations and methods provided herein. For example, with reference to SEQ ID NO: 1, which sets forth the sequence of full length precursor PH20, which includes a signal sequence (residues 1-35), soluble forms include, but are not limited to, C-terminal truncated polypeptides of human PH20 set forth in SEQ ID NO: 1 having a C-terminal amino acid residue 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO: 1, or polypeptides that exhibit at least 85%, 90%, 91%, 92%, 93%, 94%, 95%, 96%, 97%, 98%, 99%, or more sequence identity thereto, have activity at neutral pH, and are soluble (secreted into the medium when expressed in a mammalian cell). Soluble forms of human PH20 generally include those that contain amino acids 36-464 set forth in SEQ ID NO: 1. For example, when expressed in mammalian cells, the 35 amino acid N-terminal signal sequence is cleaved during processing, and the mature form of the protein is secreted. Thus, the mature soluble polypeptides include those that contain amino acids 36 to 467, 468, 469, 470, 471, 472, 473, 474, 475, 476, 477, 478, 479, 480, 481, 482 and 483 of SEQ ID NO: 1. Exemplary of soluble hyaluronidases are soluble human PH20 polypeptides that are 442, 443, 444, 445, 446 or 447 amino acids in length, such as the soluble PH20 polypeptides that have a sequence of amino acids set forth as amino acid residues 36-482, 36-477, 366-478, 36-479, 36-480, 36-481, and 36-483 of SEQ ID NO: 1, and variants thereof that retain hyaluronidase activity, and have, for example, at least 85%, 86%, 87%, 88%, 89%, 90%, 91%, 92%, 93%, 94%, 95%, 96%, 97%, 98% or 99% sequence identity to a sequence of amino acids set forth as amino acid residues 36-482, 36-477, 366-478, 36-479, 36-480, 36-481, and 36-483 of SEQ ID NO: 1. Such soluble forms of recombinant human PH20 are described, for example, in U.S. Pat. Nos. 7,767,429, 8,202,517, 8,431,380, 8,431,124, 8,450,470, 8,765,685, 8,772,246, 7,871,607, 7,846,431, 7,829,081, 8,105,586, 8,187,855, 8,257,699, 8,580,252, 9,677,061, and 9,677,062, which are incorporated by reference herein. Generally soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g. DG44 CHO cells).

Soluble recombinant human PH20 can be produced recombinantly, which includes recombinant forms of human PH20. One such product is referred to rHuPH20; rHuPH20 refers to the composition produced upon expression in a cell, such as CHO cell, of nucleic acid encoding residues 36-482 of SEQ ID NO: 1, generally linked to the native or a heterologous signal sequence (residues 1-35 of SEQ ID NO: 1). rHuPH20 is produced by expression of a nucleic acid molecule, such as encoding amino acids 1-482 (set forth in SEQ ID NO: 1). Post translational processing removes the 35 amino acid signal sequence, leaving a polypeptide or a mixture of polypeptides. As produced in the culture medium there is heterogeneity at the C-terminus such that the product, designated rHuPH20, includes a mixture of species that terminate, with reference to SEQ ID NO:1, at residues 477, 478, 479, 480, 481, and 482 in various abundance. Hyaluronidase rHuPH20 is selected from the group consisting of a polypeptides with reference to SEQ ID NO:1 corresponding to amino acid residues 36-477; amino acid residues 36-478, c amino acid residues 36-479, amino acid residues 36-480, amino acid residues 36-481, and amino acid residues 36-482. Generally, the most abundant species is the 446 amino acid polypeptide corresponding to residues 36-481 of SEQ ID NO: 1.

The soluble human PH20 polypeptides include those referred to as extended soluble hyaluronidase. Extended soluble hyaluronidases, can be produced by making C-terminal truncations to any naturally GPI-anchored hyaluronidase such that the resulting polypeptide is soluble and contains one or more amino acid residues from the GPI anchor attachment signal sequence (see, e.g., U.S. Pat. No. 8,927,249). Extended soluble human PH20 polypeptides include those that terminate at any of residues about 495-500 of SEQ ID NO:1. The mature forms start at residue 36. The extended soluble human PH20 polypeptides are neutral active, soluble. They can contain amino acid substitutions, and have at least 80%, 85%, 90%, 91%, 92%, 93%, 94%, 95% to the extended soluble PH20 polypeptides.

Hyaluronidases can be recombinantly produced or can be purified or partially-purified from natural sources, such as, for example, from testes extracts. Methods for production of recombinant proteins, including recombinant hyaluronidases, are well known in the art. Soluble PH20 is produced in cells that facilitate correct N-glycosylation to retain activity, such as CHO cells (e.g. DG44 CHO cells).

Glycosylation, including N- and O-linked glycosylation, of some hyaluronidases, including the soluble PH20 hyaluronidases, can be important for their catalytic activity and stability. For some hyaluronidases, removal of N-linked glycosylation can result in near complete inactivation of the hyaluronidase activity. N-linked oligosaccharides fall into several primary types (oligomannose, complex, hybrid, sulfated), all of which have (Man) 3-GlcNAc-GlcNAc-cores attached via the amide nitrogen of Asn residues that fall within -Asn-Xaa-Thr/Ser-sequences (where Xaa is not Pro). Glycosylation at an -Asn-Xaa-Cys-site has been reported for coagulation protein C. In some instances, a hyaluronidase, such as a PH20 hyaluronidase, can contain N-glycosidic and O-glycosidic linkages. For example, PH20 has 0-linked oligosaccharides as well as N-linked oligosaccharides. There are six potential N-linked glycosylation sites at N82, N166, N235, N254, N368, N393 of human PH20 exemplified in SEQ ID NO: 1.

In certain embodiments, the hyaluronidase enzyme is a variant or fragment of hyaluronidase PH20 having one or more amino acid residue additions, deletions or substitutions to the amino acid sequence of wild-type PH20, such as a variant or fragment hyaluronidase PH20 having one or more amino acid residue additions, deletions or substitutions to the amino acid sequence of mature wild-type PH20. In some embodiments, the variant or fragment hyaluronidase PH20 includes one or more amino acid substitutions, additions or deletions that are located in the alpha-helix 8 region of PH20. In some embodiments, the variant or fragment hyaluronidase PH20 includes one or more amino acid substitutions, additions or deletions that are located in a linker region between alpha-helix 7 and alpha-helix 8 of PH20. In certain embodiments, the variant or fragment hyaluronidase PH20 includes an amino acid sequence from L36 to S490 of wild-type PH20 (i.e., L36 to S490 of SEQ ID NO: 1)

In embodiments, the PH20 variant or fragment thereof has an amino acid sequence homology of at least 80% to the amino acid sequence of wild type PH20 enzyme of SEQ ID NO: 1, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% and including at least 99%.

In some embodiments, the variant or fragment hyaluronidase PH20 includes one or more amino acid residue substitutions at one or more positions selected from among T341 to N363, such as one or more positions selected from T341, L342, S343, I344, M345, S347, M348, K349, L352, L353, L354, D355, N356, E359, I361 and N363, but is not limited thereto. For example, the amino acid residue substitution at one or more positions selected from T341, L342, S343, I344, M345, S347, M348, K349, L352, L353, L354, D355, N356, E359, I361 and N363 may be one or more amino acid residue substitutions selected from T341A, T341C, T341D, T341G, T341S, L342W, S343E, I344N, M345T, S347T, M348K, K349E, L352Q, L353A, D355K, E359D, I361T and N363G, but is not limited thereto. In embodiments, amino acid residue substitutions are described by code numbers and letters such as “T455S”, which means the amino acid residue threonine (“T”) at the numerical position 455 of a given SEQ ID NO. is substituted with the amino acid residue serine (“S”). In certain embodiments, the variant or fragment hyaluronidase PH20 includes one or more amino acid residue substitutions selected from T341S, L342W, S343E, I344N, M345T, S347T, M348K, K349E, L352Q, L353A, L354I, D355K, N356E, E359D and I361T. In certain embodiments, the variant or fragment hyaluronidase PH20 includes one or more amino acid residue substitutions selected from M345T, S347T, M348K, K349E, L352Q, L353A, L354I, D355K, N356E, E359D and I361T, and may further include one or more amino acid residue substitutions selected from the group consisting of T341A, T341C, T341D, T341G, T341S, L342W, S343E, I344N and N363G, but is not limited thereto.

In some embodiments, the variant or fragment hyaluronidase PH20 includes one or more amino acid residue substitutions selected from: (a) T341S, L342W, S343E, I344N, M345T, S347T, M348K, K349E, L352Q, L353A, L354I, D355K, N356E, E359D and I361T; (b) L342W, S343E, I344N, M345T, S347T, M348K, K349E, L352Q, L353A, L354I, D355K, N356E, E359D and I361T; (c) M345T, S347T, M348K, K349E, L352Q, L353A, L354I, D355K, N356E, E359D, I361T and N363G; (d) T341G, L342W, S343E, I344N, M345T, S347T, M348K, K349E, L352Q, L353A, L354I, D355K, N356E, E359D and I361T; (e) T341A, L342W, S343E, I344N, M345T, S347T, M348K, K349E, L352Q, L353A, L354I, D355K, N356E, E359D and I361T; (f) T341C, L342W, S343E, I344N, M345T, S347T, M348K, K349E, L352Q, L353A, L354I, D355K, N356E, E359D and I361T; (g) T341D, L342W, S343E, I344N, M345T, S347T, M348K, K349E, L352Q, L353A, L354I, D355K, N356E, E359D and I361T; (h) I344N, M345T, S347T, M348K, K349E, L352Q, L353A, L354I, D355K, N356E, E359D and I361T; and (i) S343E, I344N, M345T, S347T, M348K, K349E, L352Q, L353A, L354I, D355K, N356E, E359D and I361T.

In some embodiments, the variant or fragment hyaluronidase PH20 includes truncation of the amino acid sequence of SEQ ID NO: 1 before F38 at the N-terminus. In some instances, the variant or fragment of hyaluronidase PH20 includes truncation of the amino acid sequence of SEQ ID NO: 1 before an amino acid residue selected from M1 to P42. For example, the variant or fragment of hyaluronidase PH20 includes truncation before amino acid residue L36, N37, F38, R39, A40, P41 or P42 at the N-terminus such that one or more amino acid residues at the N-terminus are deleted. In embodiments, the phrase truncation before an amino acid residue selected from M1 to P42 at the N-terminus is meant that an amino acid residue immediately before amino acid residue M1 to P42 at the N-terminus is cleaved and deleted.

In some embodiments, the variant or fragment hyaluronidase PH20 includes truncation of the amino acid sequence of SEQ ID NO:1 after an amino acid selected from V455 to S490. For example, the variant or fragment of hyaluronidase PH20 includes truncation of the amino acid sequence of SEQ ID NO: 1 after amino acid residue V455, C458, D461, C464, I465, D466, A467, F468, K470, P471, P472, M473, E474, T475, E476, E477, P478, Q479, I480, F481, Y482, N483, A484, P486, T488, or S490 such that one or more amino acid residues at the C-terminus are deleted. In embodiments, the phrase truncation after an amino acid residue selected from V455 to S490 at the C-terminus is meant that an amino acid residue immediately after amino acid residue V455 to S490 at the C-terminus is cleaved and deleted.

In some embodiments, the variant or fragment of hyaluronidase PH20 may have an amino acid sequence of SEQ ID NO: 1 and may include one or more amino acid residue substitutions selected from T341S, L342W, S343E, I344N, M345T, S347T, M348K, K349E, L352Q, L353A, L354I, D355K, N356E, E359D and I361T, and truncation before F38 at the N-terminus, and truncation after F468 at the C-terminus (see HP46, SEQ ID NO: 99, Table 2 below). In some instances, the variant or fragment of hyaluronidase PH20 may have an amino acid sequence of SEQ ID NO: 1 and may include one or more amino acid residue substitutions selected from T341A, T341C, T341G, S343E, M345T, K349E, L353A, L354I, N356E and I361T. In certain instances, the one or more amino acid residue substitutions are located in an alpha-helix 8 region (S347 to C381) and/or the linker region (A333 to R346) between alpha-helix 7 and alpha-helix 8. For example, the amino acid substitution in the linker region between alpha-helix 7 and alpha-helix 8 may include the substitution of one or more amino acid residues in the region consisting of amino acid residues T341 to N363, T341 to I361, L342 to I361, L342 to I361, S343 to I361, I344 to I361, M345 to I361, or M345 to N363.

In some embodiments, the variant or fragment of hyaluronidase PH20 has one or more amino acid residue substitutions located in an alpha-helix 8 region (S347 to C381) and/or the linker region (A333 to R346) between alpha-helix 7 and alpha-helix 8 of wild-type PH20 (e.g., mature wild-type PH20) and may be substituted with some amino acid residues of an amino acid sequence of a region of human HYAL1, as set forth in Tables 2 and 3, having Sequence ID NO: 2.

TABLE 2
Amino acid sequence of wild type human
HYAL1 (SEQ ID NO: 2)

MAAHLLPICALFLTLLDMAQGFRGPLLPNRPFTTVWNANTQWCLERHGVD
VDVSVFDVVANPGQTFRGPDMTIFVSSQLGTYPYYTPTGEPVFGGLPQNA
SLIAHLARTFQDILAAIPAPDFSGLAVIDWEAWRPRWAFNWDTKDIYRQR
SRALVQAQHPDWPAPQVEAVAQDQFQGAARAWMAGTLQLGRALRPRGLWG
FYGFPDCYNYDFLSPNYTGQCPSGIRAQNDQLGWLWGQSRALYPSIYMPA
VLEGTGKSQMYVQHRVAEAFRVAVAAGDPNLPVLPYVQIFYDTTNHFLPL
DELEHSLGESAAQGAAGVVLWVSWENTRTKESCQAIKEYMDTTLGPFILN
VTSGALLCSQALCSGHGSCVRRTSHPKALLLLNPASFSIQLTPGGGPLSL
RGALSLEDQAQMAVEFKCRCYPGWQAPWCERKSMW

TABLE 3
Alpha-helix and amino acid sequence comparison
between PH20 and HYAL1

| | Amino acid sequence | Amino acid sequence of |
|---|---|---|
| Alpha helix | of PH20 | HYAL1 |
| Alpha-helix 1 | P56-D65 | N39-G48 |
| Alpha-helix 3 | S119-M135 | S101-I117 |
| Alpha-helix 4’ | K161-N176 | K144-H159 |
| Alpha-helix 4 | S180-R211 | P163-R194 |
| Alpha-helix 5 | F239-S256 | P222-S239 |
| Alpha-helix 6 | A274-D293 | K257-G277 |
| Alpha-helix 7 | S317-G332 | P299-G314 |
| Alpha-helix 8 | S347-C381 | T329-C363 |

In certain embodiments, the variant or fragment of hyaluronidase PH20 has an amino acid sequence selected from SEQ ID NOS: 60 to 115, as set forth in Table 4.

TABLE 4

| Name of | | | |
|---|---|---|---|
| Variant | | | |
| or | SEQ ID | | |
| Fragment | NO. | Substitution | Amino Acid Sequence |
| HM1 | 60 | 12 amino acids | LNFRAPPVIPKVPFLWAWNAPSEFCLGKFDEPLDM |
| | | substitution of | SLFSFIGSPRINATGQGVTIFYVDRLGYYPYIDSI |
| | | M345T, S347T, | TGVTVNGGIPQKISLQDHLDKAKKDITFYMPVDNL |
| | | M348K, K349E, | GMAVIDWEEWRPTWARMWKPKDVYKNRSIELVQQQ |
| | | L352Q, L353A, | NVQLSLTEATEKAKQEFEKAGKDFLVETIKLGKLL |
| | | L354I, D355K, | RPNHLKGYYLFPDCYNHHYKKPGYNGSCFNVEIKR |
| | | N356E, E359D, | NDDLSWLWKESTALYPSIYLNTQQSPVAATLYVRN |
| | | I361T and | RVREAIRVSKIPDAKSPLPVFAYTRIVFTDQVLKF |
| | | N363G | LSQDELVYTFGETVALGASGIVIWGTLSITRTKES |
| | | | CQAIKEYMDTTLGPYIINVTLAAKMCSQVLCQEQG |
| | | | VCIRKNWKSSDYLHLNPDNFAIQLEKGGKFTVRGK |
| | | | PTLEDLEQFSEKFYCSCYSTLSCKEKADVKDTDAV |
| | | | DVCIADGVCIDAFLKPPMETEEPQIFYNASPSTLS |
| HM2 | 61 | 7 amino acids | LNFRAPPVIPNVPFLWAWNAPSEFCLGKFDEPLDM |
| | | substitution of | SLFSFIGSPRINATGQGVTIFYVDRLGYYPYIDSI |
| | | Y365F, I367L, | TGVTVNGGIPQKISLQDHLDKAKKDITFYMPVDNL |
| | | L371S, A372G, | GMAVIDWEEWRPTWARMWKPKDVYKNRSIELVQQQ |
| | | K374L, M375L | NVQLSLTEATEKAKQEFEKAGKDFLVETIKLGKLL |
| | | and V379A | RPNHLWGYYLFPDCYMKHYKKPGYNGSCFNVEIKR |
| | | | NDDLSWLWNESTALYPSIYLNTQQSPVAATLYVRN |
| | | | RVREAIRVSKIPDAKSPLPVFAYTBIVFTDQVLKF |
| | | | LSQDELVYTFGETVALGASGIVIWGTLSITRTKES |
| | | | CQAIKEYMDTTLNPFILNVTSGALLCSQALCQEQG |
| | | | VCIRKNWNSSDYLHLMPDNFAIQLEKGGKFTVRGK |
| | | | PTLEDLEQFSEKFYCSCYSTLSCKEKADVKDTDAV |
| | | | DVCIADGVCIDAFLKPPMETEEPQIFYNASPSTLS |
| HM3 | 62 | 19 amino acids | LNFRAPPVIPNVPFLWAWNAPSEFCLGKFDEPLDM |
| | | substitution of | SLFSFIGSPRINATGQGVTIFYVDRLGYYPYIDSI |
| | | M345T, S347T, | TGVTVNGGIPQKISLQDHLDKAKKDITFYMPVDNL |
| | | M348K, K349E, | GMAVIDWEEWRPTWARNWKPKDVYKKRSIELVQQQ |
| | | L352Q, L353A, | NVQLSLTEATEKAKQEFEKAGKDFLVETIKLGKLL |
| | | L354I, D355K, | RPNHLWGYYLFPDCYNHHYKKPGYNGSCFNVEIKR |
| | | N356E, E359D, | NDDLSWLWSESTALYPSIYLNTQQSPVAATLYVRN |
| | | I361T, N363G, | RVREAIRVSKIPDAKSPLPVFAYTRIVFTDQVLKF |
| | | Y365F, I367L, | LSQDELVYTFGETVALGASGIVIWGTLSITRTKES |
| | | L371S, A372G, | CQAIKEYMDTTLGPFILNVTSGALLCSQALCQEQG |
| | | K374L, M375L | VCIRKNWNSSDYLHLNPDMFAIQLEKGGKFTVRGK |
| | | and V379A | PTLEDLEQFSEKFYCSCYSTLSCKEKADVKDTDAV |
| | | | DVCIADGVCIDAFLKPPMETEEPQIFYMASPSTLS |
| HM4 | 63 | 17 amino acids | LNFRAPPVIPNVPFLWAWNAPSEFCLGKFDEPLDM |
| | | substitution of | SLFSFIGSPRINATGQGVTIFYVDRLGYYPYIDSI |
| | | G340V, T341S, | TGVTVNGGIPQKISLQDHLDKAKKDITFYMPVDNL |
| | | L342W, S343E, | GMAVIDWEEWRPTWARNWKPKDVYKNRSIELVQQQ |
| | | I344N, M345T, | NVQLSLTEATEKAKQEFEKAGKDFLVETIKLGKLL |
| | | S347T, M348K, | RPNHLWGYYLFPDCYNHHYKKPGYNGSCFNVEIKR |
| | | K349E, L352Q, | NDDLSWLWNESTALYPSIYLNTQQSPVAATLYVRN |
| | | L353A, L354I, | RVREAIRVSKIPDAKSPLPVFAYTRIVFTDQVLKF |
| | | D355K, N356E, | LSQDELVYTFGETVALGASGIVIWVSWENTRTKES |
| | | E359D, 1361T | CQAIKEYMDTTLGPYIINVTLAAKMCSQVLCQEQG |
| | | and N363G | VCIRKNWNSSDYLHLNPDNFAIQLEKGGKFTVRGK |
| | | | PTLEDLEQFSEKFYCSCYSTLSCKEKADVKDTDAV |
| | | | DVCIADGVCIDAFLKPPMETEEPQIFYNASPSTLS |
| HM6 | 64 | 11 amino acids | LNFRAPPVIPHVPFLWAWNAPSEFCLGKFDEPLCM |
| | | substitution of | SLFSFIGSPRINATGQGVTIFYVDRLGYYPYIDSI |
| | | M345T, S347T, | TGVTVNGGIPQKISLQDHLDKAKKDITFYMPVDNL |
| | | M348K, K349E, | GMAVIDWEEWRPTWARNWKPKDVYKKRSIELVQQQ |
| | | L352Q, L353A, | NVQLSLTEATEKAKQEFEKAGKDFLVETIKLGKLL |
| | | L354I, D355K, | RPNHLWGYYLFPDCYNHHYKKPGYNGSCFNVEIKR |
| | | N356E, E359D | HDDLSWLWNESTALYPSIYLNTQQSPVAATLYVRN |
| | | and I361T | RVREAIRVSKIPDAKSPLPVFAYTRIVFTDQVLKF |
| | | | LSQDELVYTFGETVALGASGIVIWGTLSITRTKES |
| | | | CQAIKEYMDTTLKPYIINVTLAAKMCSQVLCQEQG |
| | | | VCIRKNWNSSDYLHLMPDNFAIQLEKGGKFTVRGK |
| | | | PTLEDLEQFSEKFYCSCYSTLSCKEKADVKDTDAV |
| | | | DVCIADGVCIDAFLKPPMETEEPQIFYNASPSTLS |
| HM7 | 65 | 16 amino acids | LNFRAPPVIPNVPFLWAWNAPSEFCLGKFDEPLDM |
| | | substitution of | SLFSFIGSPRINATGQGVTIFYVDRLGYYPYIDSI |
| | | G340V, T341S | TGVTVNGGIPQKISLQDHLDKAKKDITFYMPVDNL |
| | | L342W, S343E, | GMAVIDWEEWRPTWARNWKPKDVYKNRSIELVQQQ |
| | | I344N, M345T, | NVQLSLTEATEKAKQEFEKAGKDFLVETIKLGKLL |
| | | S347T, M348K, | RPNHLWGYYLFPDCYNHHYKKPGYKGSCFNVEIKR |
| | | K349E, L352Q, | NDDLSWLWNESTALYPSIYLNTQQSPVAATLYVRN |
| | | L353A, L354I, | RVREAIRVSKIPDAKSPLPVFAYTRIVFTDQVLKF |
| | | D355K, N356E, | LSQDELVYTFGETVALGASGIVIWVSWENTRTKES |
| | | E359D and I361T | CQAIKEYMDTTLHPYIINVTLAAKMCSQVLCQEQG |
| | | | VCIRKNWKSSDYLHLNPDNFAIQLEKGGKFTVRGK |
| | | | PTLEDLEQFSEKFYCSCYSTLSCKEKADVKDTDAV |
| | | | DVCIADGVCIDAFLKPPMETEEPQIFYNASPSTLS |
| HM8 | 66 | 12 amino acids | LNFRAPPVIPNVPFLWAWNAPSEFCLGKFDEPLDM |
| | | substitution of | SLFSFIGSPRINATGQGVTIFYVDRLGYYPYIDSI |
| | | I344N, M345T, | TGVTVNGGIPQKISLQDHLDKAKKDITFYMPVDNL |
| | | S347T, M348K, | GMAVIDWEEWRPTWARNWKPKDVYKNRSIELVQQQ |
| | | K349E, L352Q, | NVQLSLTEATEKAKQEFEKAGKDFLVETIKLGKLL |
| | | L353A, L354I, | RPNHLWGYYLFPDCYNHHYKKPGYNGSCFNVEIKR |
| | | D355K, N356E, | NDDLSWLWNESTALYPSIYLNTQQSPVAATLYVRN |
| | | E359D and I361T | RVREAIRVSKIPDAKSPLPVFAYTRIVFTDQVLKF |
| | | | LSQDELVYTFGETVALGASGIVIWGTLSNTRTKES |
| | | | CQAIKEYMDTTLNPYIINVTLAAKMCSQVLCQEQG |
| | | | VCIRKNWKSSDYLHLNPDNFAIQLEKGGKFTVRGK |
| | | | PTLEDLEQFSEKFYCSCYSTLSCKEKADVKDTDAV |
| | | | DVCIADGVCIDAFLKPPMETEEPQIFYKASPSTLS |
| HM9 | 67 | 13 amino acids | LNFRAPPVIPNVPFLWAWNAPSEFCLGKFDEPLDM |
| | | substitution of | SLFSFIGSPRINATGQGVTIFYVDRLGYYPYIDSI |
| | | S343E, I344N, | TGVTVNGGIPQKISLQDHLDKAKKDITFYMPYDNL |
| | | M345T, S347T, | GMAVIDWEEWRPTWARNWKPKDVYKNRSIELVQQQ |
| | | M348K, K349E, | NVQLSLTEATEKAKQEFEKAGKDFLVETIKLGKLL |
| | | L352Q, L353A, | RPNHLWGYYLFPDCYNHHYKKPGYNGSCFNVEIKR |
| | | L3541, D355K, | NDDLSWLWNESTALYPSIYLNTQQSPVAATLYVRN |
| | | N356E, E359D | RVREAIRVSKIPDAKSPLPVFAYTRIVFTDQVLKF |
| | | and I361T | LSQDELVYTFGETVALGASGIVIWGTLENTRTKES |
| | | | CQAIKEYMDTTLNPYIINVTLAAKMCSQVLCQEQG |
| | | | VCIRKKWNSSDYLHLNPDNFAIQLEKGGKFTVRGK |
| | | | PTLEDLEQFSEKFYCSCYSTLSCKEKADVKDTDAV |
| | | | DVCIADGVCIDAFLKPPMETEEPQIFYNASPSTLS |
| HM10 | 68 | 14 amino acids | LNFRAPPVIPNVPFLWAWNAPSEFCLGKFDEFLDM |
| | | substitution of | SLFSFIGSPRINATGQGVTIFYVDRLGYYPYIDSI |
| | | L342W, S343E, | TGVTVNGGIPQKISLQDHLDKAKKDITFYMPVDHL |
| | | I344N, M345T, | GMAVIDWEEWRPTWARNWKPKDVYKNRSIELVQQQ |
| | | S347T, M348K, | NVQLSLTEATEKAKQEFEKAGKDFLVETIKLGKLL |
| | | K349E, L352Q, | RPNHLWGYYLFFDCYNHHYKKPGYNGSCFNVEIKR |
| | | L353A, L354I, | NDDLSWLWNESTALYPSIYLNTQQSPVAATLYVRN |
| | | D355K, N356E, | RVREAIRVSKIPDAKSPLPVFAYTRIVFTDQVLKF |
| | | E359D and I361T | LSQDELVYTFGETVALGASGIVIWGTWENTRTKES |
| | | | CQAIKEYMDTTLNPYIINVTLAAKMCSQVLCQEQG |
| | | | VCIRKNWMSSDYLHLNPDNFAIQLEKGGKFTVRGK |
| | | | PTLEDLEQFSEKFYCSCYSTLSCKEKADVKDTDAV |
| | | | DVCIADGVCIDAFLKPPMETEEPQIFYNASPSTLS |
| HM11 | 69 | 13 amino acids | LNFRAPPVIPNVPFLWAWNAPSEFCLGKFDEPLDM |
| | | substitution of | SLFSFIGSPRINATGQGVTIFYVDRLGYYPYIDSI |
| | | M345T, S347T, | TGVTVNGGIPQKISLQDHLDKAKKDITFYMPVDNL |
| | | M348K, K349E, | GMAVIDWEEWRPTWARNWKPKDVYKNRSIELVQQQ |
| | | L352Q, L353A, | NVQLSLTEATEKAKQEFEKAGKDFLVETIKLGKLL |
| | | L354I, D355K, | RPNHLWGYYLFPDCYNHHYKKPGYNGSCFNVEIKR |
| | | N356E, E359D, | NDDLSWLWNESTALYPSIYLNTQQSPVAATLYVRN |
| | | I361T, Y365F | RVREAIRVSKIPDAKSPLPVFAYTRIVFTDQVLKF |
| | | and I367L | LSQDELVYTFGETVALGASGIVIWGTLSITRTKES |
| | | | CQAIKEYMDTTLMPFILNVTLAAKMCSQVLCQEQG |
| | | | VCIRKNWMSSDYLHLNPDNFAIQLEKGGKFTVRGK |
| | | | PTLEDLEQFSEKFYCSCYSTLSCKEKADVKDTDAV |
| | | | DVCIADGVCIDAFLKPPMETEEPQIFYNASPSTLS |
| HM12 | 70 | 15 amino acids | LNFRAPPVIPNVPFLWAWNAPSEFCLGKFDEPLDM |
| | | substitution of | SLFSFIGSPRINATGQGVTIFYVDRLGYYPYIDSI |
| | | M345T, S347T, | TGVTVNGGIPQKISLQDHLDKAKKDITFYMPVDNL |
| | | M348K, K349E, | GMAVIDWEEWRPTWARNWKPKDVYKNRSIELVQQQ |
| | | L352Q, L353A, | NVQLSLTEATEKAKQEFEKAGKDFLVETIKLGKLL |
| | | L354I, D355K, | RPNHLWGYYLFPDCYNHHYKKPGYNGSCFNVEIKR |
| | | N356E, E359D, | NDDLSWLWNSSTALYPSIYLNTQQSPVAATLYVRN |
| | | I361T, Y365F, | RVREAIRVSKIPDAKSPLPVEAYTRIVFTDQVLKF |
| | | I367L, L371S | LSQDELVYTFGETVALGASGIVIWGTLSITRTKES |
| | | and A372G | CQAIKEYMDTTLNPFILNVTSGAKMCSQVLCQEQG |
| | | | VCIRKNWNSSDYLHLNPDNFAIQLEKGGKFTVRGK |
| | | | PTLEDLEQFSEKFYCSCYSTLSCKEKADVKDTDAV |
| | | | DVCIADGVCIDAFLKPPMETEEPQIFYNRSPSTLS |
| HM13 | 71 | 11 amino acids | FRAPPVIPNVPFLWAWNAPSEFCLGKFDEPLDMSL |
| | | substitution of | FSFIGSPRINATGQGVTIFYVDRLGYYPYIDSITG |
| | | M345T, S347T, | YTVNGGIPQKISLQDHLDKAKKDITFYMPVDNLGM |
| | | M348K, K349E, | AVIDWEEWRPTWARNWKPKDVYKNRSIELVQQQNV |
| | | L352Q, L353A, | QLSLTEATEKAKQEFBKAGKDFLVETIKLGKLLRP |
| | | L354I, D355K, | NHLWGYYLFPDCYNHHYKKPGYNGSCFNVEIKRND |
| | | N356E, E359D | DLSWLWNESTALYPSIYLNTQQSPVAATLYVRNRV |
| | | and I361T, | REAIRVSKIPDAKSPLPVFAYTRIVFTDQVLKFLS |
| | | and truncation | QDSLVYTFGETVALGASGIVIWGTLSITRTKESCQ |
| | | before F38 at | AIKEYMDTTLNPYIINVTLAAKMCSQVLCQEQGVC |
| | | the N-terminus | IRKNWNSSDYLHLNPDNFAIQLEKGGKFTVRGKPT |
| | | | LEDLEQFSEKFYCSCYSTLSCKEKADVKDTDAVDV |
| | | | CIADGVCIDAFLKPPMETEEPQIFYNASPSTLS |
| HM14 | 72 | 11 amino acids | LNFRAPPVIPNVPFLWAWNAPSEFCLGKFDEPLDM |
| | | substitution of | SLFSFIGSPRINATGQGVTIFYVDRLGYYPYIDSI |
| | | M345T, S347T, | TGVTVNGGIPQKISLQDHLDKAKKDITFYMPVDNL |
| | | M348K, K349E, | GMAVIDWEEWRPTWARNWKPKDVYKNRSIELVQQQ |
| | | L352Q, L353A, | NVQLSLTEATEKAKQEFEKAGKDFLVETIKLGKLL |
| | | L354I, D355K, | RPNHLWGYYLFPDCYNHHYKKPGYNGSCFNVEIKR |
| | | N356E, E359D | NDDLSWLWNESTALYPSIYLNTQQSPVAATLYVRN |
| | | and I361T | RVREAIRVSKIPDAKSPLPVFAYTRIVFTDQVLKF |
| | | and truncation | LSQDELVYTFGETVALGASGIVIWGTLSITRTKES |
| | | after I465 at | CQAIKEYMDTTLHPYIIHVTLAAKMCSQVLCQEQG |
| | | the C-terminus | VCIRKNWNSSDYLHLNPDNFAIQLEKGGKFTVRGK |
| | | | PTLEDLEQFSEKFYCSCYSTLSCKEKADVKDTDAV |
| | | | DVCIADGVCI |
| HM15 | 73 | 11 amino acids | LNFRAPPVIPNVPFLWAWNAPSEFCLGKFDEPLDM |
| | | substitution of | SLFSFIGSPRINATGQGVTIFYVDRLGYYPYIDSI |
| | | M345T, S347T, | TGVTVNGGIPQKISLQDHLDKAKKDITFYMPVDNL |
| | | M348K, K349E, | GMAVIDWEEWRPTWARNWKPKDVYKMRSIELVQQQ |
| | | L352Q, L353A, | NVQLSLTEATEKAKQEFSKAGKDFLVETIKLGKLL |
| | | L354I, D355K, | RPNHLWGYYLFPDCYNHHYKKPGYNGSCFNVEIKR |
| | | N356E, E359D | NDDLSWLWNESTALYPSIYLNTQQSPVAATLYVRN |
| | | and I361T, | RVREAIRVSKIPDAKSPLPVFAYTRIVFTDQVLKF |
| | | and truncation | LSQDELVYTFGETVALGASGIVIWGTLSITRTKES |
| | | after F468 at | CQAIKEYMDTTLNPYIINVTLAAKMCSQVLCQEQG |
| | | the C-terminus | VCIRKHWNSSDYLHLNPDNFAIQLEKGGKFTVRGK |
| | | | PTLEDLEQFSEKFYCSCYSTLSGKEKADVKDTDAV |
| | | | DVCIADGVCIDAF |
| HM16 | 74 | 11 amino acids | LNFRAPPVIPNVPFLWAWNAPSEFCLGKFDEPLDM |
| | | substitution of | SLFSFIGSPRINATGQGVTIFYVDRLGYYPYIDSI |
| | | M345T, S347T, | TGVTVNGGIPQKISLQDHLDKAKKDITFYMPVDNL |
| | | M348K, K349E, | GMAVIDWEEWRPTWARNWKPKDVYKNRSIELVQQQ |
| | | L352Q, L353A, | NVQLSLTEATEKAKQEFEKAGKDFLVETIKLGKLL |
| | | L354I, D355K, | RPNHLWGYYLFPDCYNHHYKKPGYNGSCFNVEIKR |
| | | N356E, E359D | NDDLSWLWNESTALYPSIYLNTQQSPVAATLYVRN |
| | | and I361T, | RVREAIRVSKIPDAKSPLPVFAYTRIVFTDQVLKF |
| | | and truncation | LSQDELVYTFGETVALGASGIVIWGTLSITRTKES |
| | | after P471 at | CQAIKEYMDTTLNPYIINVTLAAKMCSQVLCQEQG |
| | | the C-terminus | VCIRKNWNSSDYLHLNPDNFAIQLEKGGKFTVRGK |
| | | | PTLEDLEQFSEKFYCSCYSTLSCKEKADVKDTDAV |
| | | | DVCIADGVCIDAFLKP |
| HM17 | 75 | Substitution of | FRGPLLPNRPFLWAWNAPSEFCLGKFPEPLDMSLF |
| | | L36-V47 with | SFIGSPRINATGQGVTIFYVDRLGYYPYIDSITGV |
| | | FRGPLLPNR, and | TVNGGIPQKISLQDHLDKAKKDITFYMPVDNLGMA |
| | | amino acids | VIDWEEWRPTWARNWKPKDVYKNRSIELVQQQKVQ |
| | | substitution of | LSLTEATEKAKQEFEKAGKDFLVETIKLGKLLRPN |
| | | M345T, S347T, | HLWGYYLFPDCYNHHYKKPGYNGSCFNVEIKRNDD |
| | | M348K, K349E, | LSWLWNESTALYPSIYLNTQQSPVAATLYVRNRVR |
| | | L352Q, L353A, | EAIRVSKIPDAKSPLPVFAYTRIVFTDQVLKFLSQ |
| | | L354I, D355K, | DELVYTFGETVALGASGIVIWGTLSITPTKESCQA |
| | | N356E, E359D | IKEYMDTTLNPYIINVTLAAKMCSQVLCQEQGVCI |
| | | and I361T | RKNBNSSDYLHLNPDNFAIQLEKGGKFTVRGKPTL |
| | | | EDLEQFSEKFYCSCYSTLSCKEKADVKDTDAVDVC |
| | | | IADGVCIDAFLKPPMSTEEPQIFYNASPSTLS |
| HM18 | 76 | Substitution of | FRGPLLPNRPFTTVWNAPSEFCLGKFDEPLDMSLF |
| | | L36-A52 with | SFIGSPRINATGQGVTIFYVDRLGYYPYIDSITGV |
| | | FRGPLLPNRPFTTV, | TVNGGIPQKISLQDHLDKAKKDITFYMPVDKLGMA |
| | | and amino acids | VIDWEEWRPTWARNQKPKDVYKNRSIELVQQQNVQ |
| | | substitution of | LSLTEATEKAKQEFEKAGKDFLVETIKLGKLLRPN |
| | | M345T, S347T, | HLWGYYLFPDCYNHHYKKPGYNGSCFNVEIKRNDD |
| | | M348K, K349E, | LSWLWNESTALYPSIYLNTQQSPVAATLYVRNRVR |
| | | L352Q, L353A, | EAIRVSKIPDAKSPLPVFAYTRIVFTDQVLKFLSQ |
| | | L354I, D355K, | DELVYTFGETVALGASGIVIWGTLSITRTKESCQA |
| | | N356E, E359D | IKEYMDTTLNPYIINVTLAAKMCSQVLCQEQGVCI |
| | | and I361T | RKNWNSSDYLHLNPDNFAIQLEKGGKFTVRGKPTL |
| | | | EDLEQFSEKFYCSCYSTLSCKEKADVKDTDAVDVC |
| | | | IADGVCIDAFLKPPMETEEPQIFYNASPSTLS |
| HM19 | 77 | 14 amino acids | FRAPPVIPNVPFLWAWNAPSEFCLGKFDEPLDMSL |
| | | substitution of | FSFIGSPRINATGQGVTIFYVDRLGYYPVIDSITG |
| | | L342W, S343E, | VTVNGGIPQKISLQDHLDKAKKDITFYMPVDNLGM |
| | | I344N, M345T, | AVIDWEEWRPTWARMWKPKDVYKNRSIELVQQQNV |
| | | S347T, M348K, | QLSLTEATEKAKQEFEKAGKDFLVETIKLGKLLRP |
| | | K349E, L352Q, | NHLWGYYLFPDCYNHHYKKPGYNGSCFNVEIKRND |
| | | L353A, L354I, | DLSWLWNESTALYPSIYLNTQQSPVAATLYVRNRV |
| | | D355K, N356E, | REAIRVSKIPDAKSPLPVFAYTRIVFTDQVLKFLS |
| | | E359D and I361T, | QDELVYTFGETVALGASGIVIWGTWENTRTKESCQ |
| | | truncation | AIKEYMDTTLNPYIINVTLAAKMCSQVLCQEQGVC |
| | | before F38 at | IRKHWNSSDYLHLNPDNFAIQLEKGGKFTVRGKPT |
| | | the N-terminus, | LEDLEQFSEKFYCSCYSTLSCKEKADVKDTDAVDV |
| | | and truncation | CIADGVCIDAFLK |
| | | after K470 at | |
| | | the C-terminus | |
| HM20 | 78 | 14 amino acids | FRAPPVIPNVPFLWAWNAPSEFCLGKFDEPLDMSL |
| | | substitution of | FSFIGSPRINATGQGVTIFYVDRLGYYPYIDSITG |
| | | L342W, S343E, | VTVNGGIPQKISLQDHLDKAKKDITFYMPVDNLGM |
| | | I344N, M345T, | AVIDWEEWRPTWARNWKPKDVYKNRSIELVQQQNV |
| | | S347T, M348K, | QLSLTEATEKAKQEFEKAGKDFLVETIKLGKLLRP |
| | | K349E, L352Q, | NHLWGYYLFPDCYNHHYKKPGYNGSCFNVEIKRND |
| | | L353A, L354I, | DLSWLWNESTALYPSIYLNTQQSPVAATLYVRNRV |
| | | D355K, N356E, | REAIRVSKIPDAKSPLPVFAYTRIVFTDQVLKFLS |
| | | E359D and I361T, | QDELVYTFGETVALGASGIVIWGTWENTRTKESCQ |
| | | truncation | AIKEYMDTTLNPYIINVTLAAKMCSQVLCQEQGVC |
| | | before F38 at | IRKNWNSSDYLHLNPDNFAIQLEKGGKFTVRGKPT |
| | | the N-terminus, | LEDLEQFSEKFYCSCYSTLSCKEKADVKDTDAVDV |
| | | and truncation | CIADGVCIDAF |
| | | after F468 at | |
| | | the C-terminus | |
| HM21 | 79 | 15 amino acids | LNFRAPPVIPNVPFLWAWNAPSEFCLGKFDEPLDM |
| | | substitution of | SLFSFIGSPRINATGQGVTIFYVDRLGYVPYIDSI |
| | | T341S, L342W, | TGVTVNGGIPQKISLQDHLDKAKKDITFYMPVDNL |
| | | S343E, I344N, | GMAVIDWEEWRPTWARNWKPKDVYKNRSIELVQQQ |
| | | M345T, S347T, | HVQLSLTEATEKAKQEFEKAGKDFLVETIKLGKLL |
| | | M348K, K349E, | RPNHLWGYYLFPDCYNHHYKKPGYNGSCFNVEIKR |
| | | L352Q, L353A, | NDDLSWLWNESTALYPSIYLNTQQSPVAATLYVRN |
| | | L354I, D355K, | RVREAIRVSKIPDAKSPLPVFAYTRIVFTDQVLKF |
| | | N356E, E359D | LSQDELVYTFGETVALGASGIVIWGSWENTRTKES |
| | | and I361T | CQAIKEYMDTTLNPYIINVTLAAKMCSQVLCQEQG |
| | | | VCIRKNWNSSDYLHLNPDNFAIQLEKGGKFTVRGK |
| | | | PTLEDLEQFSEKFYCSCYSTLSCKEKADVKDTDAV |
| | | | DVCIADGVCIDAFLKPPMETEEPQIFYNASPSTLS |
| HM24 | 80 | 11 amino acids | APPVIPNVPFLWAWNAPSEFCLGKFDEPLDMSLFS |
| | | substitution of | FIGSPRINATGQGVTIFYVDRLGYYPYIDSITGVT |
| | | M345T, S347T, | VNGGIPQKISLQDHLDKAKKDITFYMPVDNLGMAV |
| | | M348K, K349E, | IDWEEWRPTWARNWKPKDVYKNRSIELVQQQNVQL |
| | | L352Q, L353A, | SLTEATEKAKQEFEKAGKDFLVETIKLGKLLRPNH |
| | | L354I, D355K, | LWGYYLFPDCYNHHYKKPGYNGSCFKVEIKRNDDL |
| | | N356E, E359D | SWLWNESTALYPSIYLNTQQSPVAATLYVBNRVRE |
| | | and I361T, and | AIRVSKIPDAKSPLPVFAYTRIVFTDQVLKFLSQD |
| | | truncation | ELVYTFGETVALGASGIVIWGTLSITRTKESCQAI |
| | | before A40 at | KEYMDTTLNPYIINVTLAAKMCSQVLCQEQGVCIR |
| | | the N-terminus | KNWNSSDYLHLNPDNFAIQLEKGGKFTVRGKPTLE |
| | | | DLEQFSEKFYCSCYSTLSCKEKADVKDTDAVDVCI |
| | | | ADGVCIDAFLKPMETEEPQIFYNASPSTLS |
| HM25 | 81 | 11 amino acids | PVIPNVPFLWAWNAPSEFCLGKFDEPLDMSLFSFI |
| | | substitution of | GSPRINATGQGVTIFYVDRLGYYPYIDSITGVTVN |
| | | M345T, S347T, | GGIPQKISLQDHLDKAKKDITFYMPVDNLGMAVID |
| | | M348K, K349E, | WEEWRPTWARNWKPKDVYKNRSIELVQQQNVQLSL |
| | | L352Q, L353A, | TEATEKAKQEFEKAGKDFLVETIKLGKLLRPNHLW |
| | | L354I, D355K, | GYYLFPDCYNHHYKKPGYNGSCFNVEIKRNDDLSW |
| | | N356E, E359D | LWNESTALYPSIYLNTQQSPVAATLYVRNRVREAI |
| | | and I361T, | RVSKIPDAKSPLPVFAYTRIVFTDQVLKFLSQDEL |
| | | truncation | VYTFGETVALGASGIVIWGTLSITRTKESCQAIKE |
| | | before P42 at | YMDTTLNPYIINVTLAAKMCSQVLCQEQGVCIRKN |
| | | the N-terminus | WNSSDYLHLNPDNFAIQLEKGGKFTVRGKPTLEDL |
| | | | BQFSEKFYCSCYSTLSCKEKADVKDTDAVDVCIAD |
| | | | GVCIDAFLKPPMETEEPQIFYNASPSTLS |
| HM29 | 82 | 14 amino acids | LNFRAPPVIPNVPFLWAWNAPSEFCLGKFDEPLDM |
| | | substitution of | SLFSFIGSPRINATGQGVTIFYVDRLGYYPYIDSI |
| | | L342W, S343E, | TGVTVNGGIPQKISLQDHLDKAKKDITFYMPVDNL |
| | | I344N, M345T, | GMAVIDWEEWRPTWARNWKPKDVYKNRSIELVQQQ |
| | | S347T, M348K, | NVQLSLTEATEKAKQEFEKAGKDFLVETIKLGKLL |
| | | K349E, L352Q, | RPNHLWGYYLFPDCYNHHYKKPGYNGSCFNVEIKR |
| | | L353A, L354I, | NDDLSWLWNESTALYPSIYLNTQQSPVAATLYVRN |
| | | D355K, N356E, | RVREAIRVSKIPDAKSPLPVFAYTRIVFTDQVLKF |
| | | E359D and I361T, | LSQDELVYTFGETVALGASGIVIWGTWENTRTKES |
| | | truncation | CQAIKEYMDTTLNPYIINVTLAAKMCSQVLCQEQG |
| | | before L36 at | VCIRKNWNSSDYLHLNPDNFAIQLEKGGKFTVRGK |
| | | the N- terminus, | PTLEDLEQFSEKFYCSCYSTLSCKEKADVKDTDAV |
| | | and truncation | DVCIADGVCIDA |
| | | after A467 at | |
| | | the C-terminus | |
| HM30 | 83 | 14 amino acids | LNFRAPPVIPNVPFLWAWNAPSEFCLGKFDEPLDM |
| | | substitution of | SLFSFIGSPRINATGQGVTIFYVDRLGYYPYIDSI |
| | | L342W, S343E, | TGVTVNGGIPQKISLQDHLDKAKKDITFYMPVDNL |
| | | I344N, M345T, | GMAVIDWEEWRPTWARNWKPKDVYKNRSIELVQQQ |
| | | S347T, M348K, | NVQLSLTEATEKAKQEFEKAGKDFLVETIKLGKLL |
| | | K349E, L352Q, | RPNHLWGYYLFPDCYNHHYKKPGYNGSCFNVEIKR |
| | | L353A, L354I, | NDDLSWLWNESTALYPSIYLNTQQSPVAATLYVRN |
| | | D355K, N356E, | RVREAIRVSKIPDAKSPLPVFAYTRIVFTDQVLKF |
| | | E359D and | LSQDELVYTFGETVALGASGIVIWGTWENTRTKES |
| | | I361T, | CQAIKEYMDTTLNPYIINVTLAAKMCSQVLCQEQG |
| | | truncation | VCIRKNWNSSDYLHLNPDNFAIQLEKGGKFTVRGK |
| | | before L36 at | PTLEDLEQFSEKFYCSCYSTLSCKEKADVKDTDAV |
| | | the N-terminus, | DVCIADGVC |
| | | and truncation | |
| | | after C464 at | |
| | | the C-terminus | |
| HM31 | 84 | 14 amino acids | LNFRAPPVIPNVPFLWAWNAPSEFCLGKFDEPLDM |
| | | substitution of | SLFSFIGSPRINATGQGVTIFYVDRLGYYFYIDSI |
| | | L342W, S343E, | TGVTVNGGIPQKISLQDHLDKAKKDITFYMPVDNL |
| | | I344N, M345T, | GMAVIDWEEWRPTWARNWKPKDVYKNRSIELVQQQ |
| | | S347T, M348K, | NVQLSLTEATEKAKQEFEKAGKDFLVETIKLGKLL |
| | | K349E, L352Q, | RPNHLWGYYLFPDCYNHHYKKPGYNGSCFNVEIKR |
| | | L353A, L354I, | NDDLSWLWNESTALYPSIYLNTQQSPVAATLYVRN |
| | | D355K, N356E, | RVREAIRVSKIPDAKSPLPVFAYTRIVFTDQVLKF |
| | | E359D and I361T, | LSQDELVYTFGSTVALGASGIVIWGTWENTRTKES |
| | | truncation | CQAIKEYMDTTLNPYIINVTLAAKMCSQVLCQEQG |
| | | before L36 at | VCIRKNWNSSDYLHLNPDNFAIQLEKGGKFTVRGK |
| | | the N-terminus, | PTLEDLEQFSEKFYCSCYSTLSCKEKADVKDTDAV |
| | | and truncation | DVCIAD |
| | | after D461 at | |
| | | the C-terminus | |
| HM32 | 85 | 14 amino acids | LNFRAPPVIPNVPFLWAWNAPSEFCLGKFDEPLDM |
| | | substitution of | SLFSFIGSPRINATGQGVTIFYVDRLGYYPYIDSI |
| | | L342W, S343E, | TGVTVNGGIPQKISLQDHLDKAKKDITFYMPVDKL |
| | | I344N, M345T, | GMAVIDWEEWRPTWARNWKPKDVYKNRSIELVQQQ |
| | | S347T, M348K, | NVQLSLTEATEKAKQEFEKAGKDFLVETIKLGKLL |
| | | K349E, L352Q, | RPNHLWGYYLFPDCYNHHYKKPGYNGSCFNVEIKR |
| | | L353A, L354I, | NDDLSWLWNESTALYPSIYLNTQQSPVAATLYVRN |
| | | D355K, N356E, | RVREAIRVSKIPDAKSPLPVFAYTRIVFTDQVLKF |
| | | E359D and | LSQDELVYTFGETVALGASGIVIWGTWENTRTKES |
| | | I361T, | CQAIKEYMDTTLNPYIINVTLAAKMCSQVLCQEQG |
| | | truncation | VCIRKNWNSSDYLHLNPDNFAIQLEKGGKFTVRGK |
| | | before L36 at | PTLEDLEQFSEKFYCSCYSTLSCKEKADVKDTDAV |
| | | the N-terminus, | DVC |
| | | and truncation | |
| | | after C458 at | |
| | | the C-terminus | |
| HM33 | 86 | 14 amino acids | LNFRAPPVIPNVPFLWAWNAPSEFCLGKFDEPLDM |
| | | substitution of | SLFSFIGSPRINATGQGVTIFYVDRLGYYPYIDSI |
| | | L342W, S343E, | TGVTVMGGIPQKISLQDHLDKAKKDITFYMPVDNL |
| | | I344N, M345T, | GMAVIDWEEWRPTWARNWKPKDVYKNRSIELVQQQ |
| | | S347T, M348K, | NVQLSLTEATEKAKQEFEKAGKDFLVETIKLGKLL |
| | | K349E, L352Q, | RPNHLWGYYLFPDCYNHHYKKPGYNGSCFNVEIKR |
| | | L353A, L354I, | NDDLSWLWNESTALYPSIYLNTQQSPVAATLYVRN |
| | | D355K, N356E, | RVREAIRVSKIPDAKSPLPVFAYTRIVFTDQVLKF |
| | | E359D and I361T, | LSQDELVYTFGETVALGASGIVIWGTWENTRTKES |
| | | truncation | CQAIKEYMDTTLNPYIINVTLAAKMCSQVLCQEQG |
| | | before L36 at | VCIRKNWNSSDYLHLNPDNFAIQLEKGGKFTVRGK |
| | | the N-terminus, | PTLEDLEQFSEKFYCSCYSTLSCKEKADVKDTDAV |
| | | and truncation | |
| | | after V455 at | |
| | | the C-terminus | |
| HP34 | 87 | 15 amino acids | FRAPPVIPNVPFLWAWNAPSEFCLGKFDEPLDMSL |
| | | substitution of | FSFIGSPRINATGQGVTIFYVDRLGYYPYIDSITG |
| | | T341S, L342W, | VTVNGGIPQKISLQDHLDKAKKDITFYMPVDNLGM |
| | | S343E, 1344N, | AVIDWEEWRPTWARNWKPKDVYKNRSIELVQQQNV |
| | | M345T, S347T, | QLSLTEATEKAKQEFEKAGKDFLVETIKLGKLLRP |
| | | M348K, K349E, | NHLWGYYLFPDCYNHHYKKPGYNGSCFNVEIKRND |
| | | L352Q, L353A, | DLSWLWNESTALYPSIYLMTQQSPVAATLYVRNRV |
| | | L354I, D355K, | REAIRVSKIPDAKSPLPVFAYTRIVFTDQVLKFLS |
| | | N356E, E359D | QDELVYTFGETVALGASGIVIWGSWENTRTKESCQ |
| | | and I361T, | AIKEYMDTTLHPYIINVTLAAKMCSQVLCQEQGVC |
| | | truncation | IRKNWNSSDYLHLNPDNFAIQLEKGGKFTVRGKPT |
| | | before F38 at | LEDLEQFSEKFYCSCYSTLSCKEKADVKDTDAVDV |
| | | the N-terminus, | CIADGVCIDAFLK |
| | | and truncation | |
| | | after K470 at | |
| | | the C-terminus | |
| HM35 | 88 | 14 amino acids | FRAPPVIPNVPFLWAWNAPSEFCLGKFDEPLDMSL |
| | | substitution of | FSFIGSPRINATGQGVTIFYVDRLGYYPYIDSITG |
| | | L342W, S343E, | VTVNGGIPQKISLQDHLDKAKKDITFYMPVDNLGM |
| | | I344N, M345T, | AVIDWEEWRPTWARNWKPKDVYKNRSIELVQQQMV |
| | | S347T, M348K, | QLSLTEATSKAKQEFEKAGKDFLVETIKLGKLLRP |
| | | K349E, L352Q, | NHLWGYYLFPDCYNHHYKKPGYNGSCFNVEIKRND |
| | | L353A, L354I, | DLSWLWNESTALYPSIYLNTQQSPVAATLYVRNRV |
| | | D355K, N356E, | REAIRVSKIPDAKSPLPVFAYTRIVFTDQVLKFLS |
| | | E359D and | QDELVYTFGETVALGASGIVIWGTWENTRTKESCQ |
| | | I361T, | AIKEYMDTTLNPYIINVTLAAKMCSQVLCQEQGVC |
| | | truncation | IRKNWNSSDYLHLNPDNFAIQLEKGGKFTVRGKPT |
| | | before F38 at | LEDLEQFSEKFYCSCYSTLSCKEKADVKDTDAVDV |
| | | the N-terminus, | CIADGVCIDAFLKPP |
| | | and truncation | |
| | | after P472 at | |
| | | the C-terminus | |
| HM36 | 89 | 14 amino acids | FRAPPVIPNVPFLWAWNAPSEFCLGKFDEPLDMSL |
| | | substitution of | FSFIGSPRINATGQGVTIFYVDRLGYYPYIDSITG |
| | | L342W, S343E, | VTVNGGIPQKISLQDHLDKAKKDITFYMPVDNLGM |
| | | I344N, M345T, | AVIDWEEWRPTWARNWKPKDVYKNRSIELVQQQNV |
| | | S347T, M348K, | QLSLTEATEKAKQEFEKAGKDFLVETIKLGKLLRP |
| | | K349E, L352Q, | NHLWGYYLFPDCYNHHYKKPGYNGSCFNVEIKRND |
| | | L353A, L354I, | DLSWLWNESTALYPSIYLNTQQSPVAATLYVRNRV |
| | | D355K, N356E, | REAIRVSKIPDAKSPLPVFAYTRIVFTDQVLKFLS |
| | | E359D and I361T, | QDELVYTFGETVALGASGIVIWGTWENTRTKESCQ |
| | | truncation | AIKEYMDTTLNPYIINVTLAAKMCSGVLCQEQGVC |
| | | before F38 at | IRKNWNSSDYLHLNPDNFAIQLEKGGKFTVRGKPT |
| | | the N-terminus, | LEDLEQFSEKFYCSCYSTLSCKEKADVKDTDAVDV |
| | | and truncation | CIADGVCIDAFLKPPM |
| | | after M473 at | |
| | | the C-terminus | |
| HM37 | 90 | 14 amino acids | FRAPPVIPNVPFLWAWNAPSEFCLGKFDEPLDMSL |
| | | substitution of | FSFIGSPRINATGQGVTIFYVDRLGYYPYIDSITG |
| | | L342W, S343E, | VTVNGGIPQKISLQDHLDKAKKDITFYMPVDNLGM |
| | | I344N, M345T, | AVIDWEEWRPTWARNWKPKDVYKNRSIELVQQQNV |
| | | S347T, M348K, | QLSLTEATEKAKQEFEKAGKDFLVSTIKLGKLLRF |
| | | K349E, L352Q, | NHLWGYYLFPDCYNHHYKKPGYMGSCFNVEIKRND |
| | | L353A, L354I, | DLSWLWNESTALYPSIYLNTQQSPVAATLYVRNRV |
| | | D355K, N356E, | REAIRVSKIPDAKSPLPVFAYTRIVFTDQVLKFLS |
| | | E359D and | QDELVYTFGETYALGASGIVIWGTWENTRTKESCQ |
| | | I361T, | AIKEYMDTTLNPYIINVTLAAKMCSQVLCQEQGVC |
| | | truncation | IRKMWNSSDYLHLNPDNFAIQLEKGGKFTVRGKPT |
| | | before F38 at | LEDLEQFSEKFYCSCYSTLSCKEKADVKDTDAVDV |
| | | the N-truncation | CIADGVCIDAFLKPPME |
| | | after E474 at | |
| | | the C-terminus | |
| HM38 | 91 | 14 amino acids | FRAPPVIPNVPFLWAWNAPSEFCLGKFDEPLDMSL |
| | | substitution of | FSFIGSPRINATGQGVTIFYVDRLGYYPYIDSITG |
| | | L342W, S343E, | VTVNGGIPQKISLQDHLDKAKKDITFYMPVDNLGM |
| | | I344N, M345T, | AVIDWEEWRPTWARNWKPKDVYKMRSIELVQQQNV |
| | | S347T, M348K, | QLSLTEATEKAKQEFEKAGKDFLVETIKLGKLLRP |
| | | K349E, L352Q, | NHLWGYYLFPDCYNHHYKKPGYNGSCFNVEIKRND |
| | | L353A, L354I, | DLSWLWNESTALYPSIYLNTQQSPVAATLYVRNRV |
| | | D355K, N356E, | REAIRVSKIPDAKSPLPVFAYTRIVFTDQVLKFLS |
| | | E359D and | QDELVYTFGETVALGASGIVIWGTWENTRTKESCQ |
| | | I361T, | AIKEYMDTTLNPYIINVTLAAKMCSQVLCQEQGVC |
| | | truncation | IRKNWNSSDYLHLNPDNFAIQLEKGGKFTVRGKPT |
| | | before F38 at | LEDLEQFSEKFYCSCYSTLSCKEKADVKDTDAVDV |
| | | the N-terminus, | CIADGVCIDAFLKPPMET |
| | | and truncation | |
| | | after T475 at | |
| | | the C-terminus | |
| HM39 | 92 | 14 amino acids | FRAPPVIPNVPFLWAWNAPSEPCLGKFDEPLDMSL |
| | | substitution of | FSFIGSPRINATGQGVTIFYVDRLGYYPYIDSITG |
| | | L342W, S343E, | VTVNGGIPQKISLQDHLDKAKKDITFYMPVDNLGM |
| | | 1344N, M345T, | AVIDWEEWRPTWARNWKPKDVYKNRSIELVQQQNV |
| | | S347T, M348K, | QLSLTEATEKAKQEFEKAGKDFLVETIKLGKLLRP |
| | | K349E, L352Q, | NHLWGYYLFPDCYNHHYKKPGYNGSCFNVEIKRND |
| | | L353A, L354I, | DLSWLWNESTALYPSIYLNTQQSPVAATLYVRNRV |
| | | D355K, N356E, | REAIRVSKIPDAKSPLPVFAYTRIVFTDQVLKFLS |
| | | E359D and I361T, | QDELVYTFGETVALGASGIVIWGTWENTRTKESCQ |
| | | truncation | AIKEYMDTTLMPYIINVTLAAKMCSQVLCQEQGVC |
| | | before F38 at | IRKNWNSSDYLHLNPDNFAIQLEKGGKFTVRGKPT |
| | | the N-terminus, | LEDLEQFSEKFYCSCYSTLSCKEKADVKDTDAVDV |
| | | and truncation | CIADGVCIDAFLKPPMETE |
| | | after E476 at | |
| | | the C-terminus | |
| HM40 | 93 | 11 amino acids | NFRAPPVIPKVPFLWAWNAPSEFCLGKFDEPLDMS |
| | | substitution of | LFSFIGSPRINATGQGVTIFYVDRLGYYPYIDSIT |
| | | M345T, S347T, | GVTVNGGIPQKISLQDHLDKAKKDITFYMPVDNLG |
| | | M348K, K349E, | MAVIDWEEWRPTWARNWKPKDVYKNRSIELVQQQN |
| | | L352Q, L353A, | VQLSLTEATEKAKQEFEKAGKDFLVETIKLGKLLR |
| | | L354I, D355K, | PNHLWGYYLFPDCYNHHYKKPGYNGSCFNVEIKRN |
| | | N356E, E359D and | DDLSWLWNESTALYPSIYLNTQQSPVAATLYVRNR |
| | | I361T, and | VREAIRVSKIPDAKSPLPVFAYTRIVFTDQVLKFL |
| | | truncation | SQDELVYTFGETVALGASGIVIWGTLSITRTKESC |
| | | before N37 at | QAIKEYMDTTLNPYIINVTLAAKMCSQVLCQEQGV |
| | | the N-terminus | CIRKNWNSSDYLHLMPDNFAIQLEKGGKFTVRGKP |
| | | | TLEDLEQFSEKFYCSCYSTLSCKEKADVKDTDAVD |
| | | | VCIADGVCIDAFLKPPMETEEPQIFYHASPSTLS |
| HM41 | 94 | 11 amino acids | RAPPVIPNVPFLWAWNAPSEFCLGKFDEPLDMSLF |
| | | substitution of | SFIGSPRINATGQGVTIFYVDRLGYYPYIDSITGV |
| | | M345T, S347T, | TVNGGIPQKISLQDHLDKAKKDITFYMPVDNLGMA |
| | | M348K, K349E, | VIDWEEWRPTWARNWKPKDVYKNRSIELVQQQNVQ |
| | | L352Q, L353A, | LSLTEATEKAKQEFEKAGKDFLVETIKLGKLLRPN |
| | | L354I, D355K, | HLWGYYLFPDCYNHHYKKPGYNGSCFNVEIKRNDD |
| | | N356E, E359D | LSWLWNESTALYPSIYLNTQQSPVAATLYVRNRVR |
| | | and I361T, | EAIRVSKIPDAKSPLPVFAYTRIVFTDQVLKFLSQ |
| | | and truncation | DELVYTFGETVALGASGIVIWGTLSITRTKESCQA |
| | | before R39 at | IKEYMDTTLNPYIINVTLAAKMCSQVLCQEQGVCI |
| | | the N-terminus | RKNWNSSDYLHLNPDNFAIQLEKGGKFTVRGKPTL |
| | | | EDLEQFSEKFYCSCYSTLSCKEKADVKDTDAVDVC |
| | | | IADGVCIDAFLKPPMETEEPQIFYNASPSTLS |
| HM42 | 95 | 11 amino acids | PPVIPNVPFLWAWNAPSEFCLGKFDEPLDMSLPSF |
| | | substitution of | IGSPRINATGQGVTIFYVDRLGYYPYIDSITGVTV |
| | | M345T, S347T, | NGGIPQKISLQDHLDKAKKDITFYMPVDNLGMAVI |
| | | M348K, K349E, | DWEEWRPTWARNWKPKDVYKNRSIELVQQQNVQLS |
| | | L352Q, L353A, | LTEATEKAKQEFEKAGKDFLVETIKLGKLLRPNHL |
| | | L354I, D355K, | WGYYLFPDCYNHHYKKPGYNGSCFNVEIKRNDDLS |
| | | N356E, E359D | WLWNESTALYPSIYLNTQQSPVAATLYVRNRVREA |
| | | and I361T, | IRVSKIPDAKSPLPVFAYTRIVFTDQVLKFLSQDE |
| | | and truncation | LVYTFGETVALGASGIVIWGTLSITRTKESCQAIK |
| | | before P41 at | EYMDTTLNPYIINVTLAAKMCSQVLCQEQGVCIRK |
| | | the N-terminus | NWNSSDYLHLNPDNFAIQLEKGGKFTVRGKPTLED |
| | | | LEQFSEKFYCSCYSTLSCKEKADVKDTDAVDVCIA |
| | | | DGVCIDAFLKPPMETEEPQIFYNASPSTLS |
| HM43 | 96 | 14 amino acids | FRAPPVIPNVPFLWAWNAPSEFCLGKFDEPLDMSL |
| | | substitution of | FSFIGSPRINATGQGVTIFYVDRLGYYPYIDSITG |
| | | L342W, S343E, | VTVNGGIPQKISLQDHLDKAKKDITFYMPVDNLGM |
| | | I344N, M345T, | AVIDWEEWRPTWARNWKPKDVYKNRSIELVQQQNV |
| | | S347T, M348K, | QLSLTEATEKAKQEFEKAGKDFLVETIKLGKLLRP |
| | | K349E, L352Q, | NHLWGYYLFPDCYNHHYKKPGYNGSCFNVEIKRND |
| | | L353A, L354I, | DLSWLWNESTALYPSIYLNTQQSPVAATLYVRNRV |
| | | D355K, N356E, | REAIRVSKIPDAKSPLPVFAYTRIVFTDQVLKFLS |
| | | E359D and I361T, | QDELVYTFGETVALGASGIVIWGSWENTRTKESCQ |
| | | truncation | AIKEYMDTTLNPYIINVTLAAKMCSQVLCQEQGVC |
| | | before F38 at | IRKNWMSSDYLHLHPDNFAIQLEKGGKFTVRGKPT |
| | | the N-terminus, | LEDLEQFSEKFYCSCYSTLSCKEKADVKDTDAVDV |
| | | and truncation | CIADGVCI |
| | | after I465 at | |
| | | the C-terminus | |
| HM44 | 97 | 14 amino acids | FRAPPVIPNVPFLWAWNAPSEFCLGKFDEPLDMSL |
| | | substitution of | FSFIGSPRINATGQGVTIFYVDRLGYYPYIDSITG |
| | | L342W, S343E, | VTVNGGIPQKISLQDHLDKAKKDITFYMPVDNLGM |
| | | I344N, M345T, | AVIDWEEWRPTWARNWKPKDVYKNRSIELVQQQNV |
| | | S347T, M348K, | QLSLTEATEKAKQEFEKAGKDFLVETIKLGKLLRP |
| | | K349E, L352Q, | NHLWGYYLFPDCYNHHYKKPGYNGSCFNVEIKRND |
| | | L353A, L354I, | DLSWLWNESTALYPSIYLNTQQSPVAATLYVRMRV |
| | | D355K, N356E, | REAIRVSKIPDAKSFLPVFAYTRIVFTDQVLKFLS |
| | | E359D and I361T, | QDELVYTFGETVALGASGIVIWGSWENTRTKESCQ |
| | | truncation | AIKEYMDTTLNPYIINVTLAAKMCSQVLCQEQGVC |
| | | before F38 at | IRKNWNSSDYLHLNPDNFAIQLEKGGKFTVRGKPT |
| | | the N-terminus, | LEDLEQFSEKFYCSCYSTLSCKEKADVKDTDAVDV |
| | | and truncation | CIADGVCID |
| | | after D466 at | |
| | | the C-terminus | |
| HM45 | 98 | 14 amino acids | FRAPPVIPNVPFLWAWNAPSEFCLGKFDEPLDMSL |
| | | substitution of | FSFIGSPRINATGQGVTIFYVDRLGYYPYIDSITG |
| | | L342W, S343E, | VTVNGGIPQKISLQDHLDKAKKDITFYMPVDWLGM |
| | | I344N, M345T, | AVIDWEEWRPTWARNWKPKDVYKNRSIELVQQQNV |
| | | S347T, M348K, | QLSLTEATSKAKQEFEKAGKDFLVETIKLGKLLRP |
| | | K349E, L352Q, | NHLWGYYLFPDCYNHHYKKPGYNGSCFNVEIKRND |
| | | L353A, L354I, | DLSWLWNESTALYPSIYLNTQQSPVAATLYVRNRV |
| | | D355K, N356E, | REAIRVSKIPDAKSPLPVFAYTRIVFTDQVLKFLS |
| | | E359D and I361T, | QDELVYTFGETVALGASGIVIWGSWENTRTKESCQ |
| | | truncation | AIKEYMDTTLNPYIINVTLAAKMCSQVLCQEQGVC |
| | | before F38 at | IRKNWNSSDYLHLNPDHFAIQLEKGGKFTVRGKPT |
| | | the N-terminus, | LEDLEQFSEKFYCSCYSTLSCKEKADVKDTDAVDV |
| | | and truncation | CIADGVCIDA |
| | | after A467 at | |
| | | the C-terminus | |
| HP46 | 99 | 15 amino acids | FRAPPVIPNVPFLWAWNAPSEFCLGKFDEPLDMSL |
| | | substitution of | FSFIGSPRINATGQGVTIFYVDRLGYYPYIDSITG |
| | | T341S, L342W, | VTVHGGIPQKISLQDHLDKAKKDITFYMPVDNLGM |
| | | S343E, I344N, | AVIDWEEWRPTWARNWKPKDVYKKRSIELVQQQNV |
| | | M345T, S347T, | QLSLTEATEKAKQEFEKAGKDFLVETIKLGKLLRP |
| | | M348K, K349E, | NHLWGYYLFPDCYNHHYKKPGYNGSCFNVEIKRND |
| | | L352Q, L353A, | DLSWLWNESTALYPSIYLNTQQSPVAATLYVRNRV |
| | | L354I, D355K, | REAIRVSKIPDAKSPLPVFAYTRIVFTDQVLKFLS |
| | | N356E, E359D | QDELVYTFGETVALGASGIVIWGSWENTRTKESCQ |
| | | and I361T, | AIKEYMDTTLNPYIINVTLAAKMCSQVLCQEQGVC |
| | | truncation | IRKNWNSSDYLHLNPDNFAIQLEKGGKFTVRGKPT |
| | | before F38 at | LEDLEQFSEKFYCSCYSTLSCKEKADVKDTDAVDV |
| | | the N-terminus, | CIADGVCIDAF |
| | | and truncation | |
| | | after F468 at | |
| | | the C-terminus | |
| HM47 | 100 | 14 amino acids | FRAPPVIPNVPFLWAWNAFSEFCLGKFDEPLDMSL |
| | | substitution of | FSFIGSPRINATGQGVTIFYVDRLGYYPYIDSITG |
| | | L342W, S343E, | VTVNGGIPQKISLQDHLDKAKKDITFYMPVDNLGM |
| | | I344N, M345T, | AVIDWEEWRPTWARNWKPKDVYKNRSIELVQQQNV |
| | | S347T, M348K, | QLSLTEATEKAKQEFSKAGKDFLVETIKLGKLLRP |
| | | K349E, L352Q, | NHLWGYYLFPDCYNHHYKKPGYNGSCFKVEIKRND |
| | | L353A, L354I, | DLSWLWNESTALYPSIYLNTQQSPVAATLYVRNRV |
| | | D355K, N356E, | REAIRVSKIPDAKSPLPVFAYTRIVFTDQVLKFLS |
| | | E359D and I361T, | QDELVYTFGETVALGASGIVIWGTWENTRTKESCQ |
| | | truncation | AIKEYMDTTLNPYIINVTLAAKMCSQVLCQEQGVC |
| | | before F38 at | IRKNWNSSDYLHLNPDNFAIQLEKGGKFTVRGKPT |
| | | the N-terminus, | LEDLEQFSEKFYCSCYSTLSCKEKADVKDTDAVDV |
| | | and truncation | CIADGVCIDAFLKPPMETEEP |
| | | after P478 at | |
| | | the C-terminus | |
| HM48 | 101 | 14 amino acids | FRAPPVIPNVPFLWAWNAPSEFCLGKFDEPLDMSL |
| | | substitution of | FSFIGSPRINATGQGVTIFYVDRLGYYPYIDSITG |
| | | L342W, S343E, | VTVNGGIPQKISLQDHLDKAKKDITFYMPVDNLGM |
| | | I344N, M345T, | AVIDWEEWRFTWARNWKPKDVYKNRSIELVQQQNV |
| | | S347T, M348K, | QLSLTEATEKAKQEFEKAGKDFLVETIKLGKLLRP |
| | | K349E, L352Q, | NHLWGYYLFPDCYNHHYKKPGYNGSCFNVEIKRND |
| | | L353A, L354I, | DLSWLWNESTALYPSIYLNTQQSPVAATLYVRNRV |
| | | D355K, N356E, | REAIRVSKIPDAKSPLPVFAYTRIVFTDQVLKFLS |
| | | E359D and I361T, | QDELVYTFGETVALGASGIVIWGTWENTRTKESCQ |
| | | truncation | AIKEYMDTTLNPYIINVTLAAKMCSQVLCQEQGVC |
| | | before F38 at | IRKNWNSSDYLHLNPDNFAIQLEKGGKFTVRGKPT |
| | | the N-terminus, | LEDLEQFSEKFYCSCYSTLSCKEKADVKPTDAVDV |
| | | and truncation | CIADGVCIDAFLKPPMETBBPQI |
| | | after I480 at | |
| | | the C-terminus | |
| HM49 | 102 | 14 amino acids | FRAPPVIPNVPFLWAWNAPSEFCLGKFDEPLDMSL |
| | | substitution of | FSFIGSPRINATGQGVTIFYVDALGYYPYIDSITG |
| | | L342W, S343E, | VTVNGGIPQKISLQDHLDKAKKDITFYMPVDNLGM |
| | | I344N, M345T, | AVIDWEEWRPTWARNWKPKDVYKNRSIELVQQQNV |
| | | S347T, M348K, | QLSLTEATEKAKQEFEKAGKDFLVETIKLGKLLRP |
| | | K349E, L352Q, | NHLWGYYLFPDCYNHHYKKPGYNGSCFNVEIKRND |
| | | L353A, L354I, | DLSWLWNESTALYPSIYLNTQQSPVAATLYVRNRV |
| | | D355K, N356E, | REAIRVSKIPDAKSPLPVFAYTRIVFTDQVLKFLS |
| | | E359D and I361T, | QDELVYTFGETVALGASGIVIWGTWENTRTKESCQ |
| | | truncation | AIKEYMDTTLNPYIINVTLAAKMCSQVLCQEQGVC |
| | | before F38 at | IRKNWNSSDYLHLNPDNFAIQLSKGGKFTVRGKPT |
| | | the N-terminus, | LEDLEQFSEKFYCSCYSTLSCKEKADVKDTDAVDV |
| | | and truncation | CIADGVCIDAFLKPPMETEEPQIFY |
| | | after Y482 at | |
| | | the C-terminus | |
| HM50 | 103 | 14 amino acids | FRAPPVIPNVPFLWAWNAPSEFCLGKFDEPLDMSL |
| | | substitution of | FSFIGSPRINATGQGVTIFYVDRLGYYPYIDSITG |
| | | L342W, S343E, | VTVNGGIPQKISLQDHLDKAKKDITFYMPVDNLGM |
| | | I344N, M345T, | AVIDWEEWRPTWARNWKPKDVYKNRSIELVQQQNV |
| | | S347T, M348K, | QLSLTEATSKAKQEFEKAGKDFLVSTIKLGKLLRP |
| | | K349E, L352Q, | NHLWGYYLFPDCYNHHYKKPGYNGSCFNVEIKRND |
| | | L353A, L354I, | DLSWLWNESTALYPSIYLNTQQSPVAATLYVRNRV |
| | | D355K, N356E, | REAIRVSKIPDAKSPLPVFAYTRIVFTDQVLKFLS |
| | | E359D and I361T, | QDELVYTFGETVALGASGIVIWGTWENTRTKESCQ |
| | | truncation | AIKEYMDTTLNPYIINVTLAAKMCSQVLCQEQGVC |
| | | before F38 at | IRKNWNSSDYLHLNPDNFAIQLEKGGKFTYRGKPT |
| | | the N-terminus, | LEDLEQFSEKFYCSCYSTLSCKEKADVKDTDAVDV |
| | | and truncation | CIADGVCIDAFLKPPMETEEPQIFYNA |
| | | after A484 at | |
| | | the C-terminus | |
| HM51 | 104 | 14 amino acids | FRAPPVIPNVPFLWAWNAPSEFCLGKFDEPLDMSL |
| | | substitution of | FSFIGSPRINATGQGVTIFYVDRLGYYPYIDSITG |
| | | L342W, S343E, | VTVNGGIPQKISLQDHLDKAKKDITFYMPVDNLGM |
| | | I344N, M345T, | AVIDWEEWRPTWARNWKPKDVYKNRSIELVQQQNV |
| | | S347T, M348K, | QLSLTEATSKAKQEFEKAGKDFLVETIKLGKLLRP |
| | | K349E, L352Q, | NHLWGYYLFPDCYNHHYKKPGYNGSCFEVEIKRND |
| | | L353A, L354I, | DLSWLWNESTALYPSIYLNTQQSPVAATLYVRNRV |
| | | D355K, N356E, | REAIRVSKIPDAKSPLPVFAYTRIVFTDQVLKFLS |
| | | E359D and I361T, | QDELVYTFGETVALGASGIVIWGTWENTRTKESCQ |
| | | truncation | AIKEYMDTTLNPYIINVTLAAKMCSQVLCQEQGVC |
| | | before F38 at | IRKNWNSSDYLHLNPDNFAIQLEKGGKFTVRGKPT |
| | | the N-terminus, | LEDLEQFSEKFYCSCYSTLSCKEKADVKDTDAVDV |
| | | and truncation | CIADGVCIDAFLKPPMETEEPQIFYNASP |
| | | after P486 at | |
| | | the C-terminus | |
| HM52 | 105 | 14 amino acids | FRAPPVIPNVPFLWAWNAPSEFCLGKFDEPLDMSL |
| | | substitution of | FSFIGSPRINATGQGVTIFYVDRLGYYFYIDSITG |
| | | L342W, S343E, | VTVNGGIFQKISLQDHLDKAKKDITFYMPVDNLGM |
| | | I344N, M345T, | AVIDWEEWRPTWARNWKPKDVYKNRSIELVQQQNY |
| | | S347T, M348K, | QLSLTEATEKAKQEFEKAGKDFLVETIKLGKLLRP |
| | | K349E, L352Q, | NHLKGYYLFPDCYNHHYKKPGYNGSCFNVEIKRND |
| | | L353A, L354I, | DLSWLWNESTALYPSIYLNTQQSPVAATLYVRNRV |
| | | D355K, N356E, | REAIRVSKIPDAKSPLPVFAYTRIVFTDQVLKFLS |
| | | E359D and I361T, | QDELVYTFGETVALGASGIVIWGTWENTRTKESCQ |
| | | truncation | AIKEYMDTTLNPYIINVTLAAKMCSQVLCQEQGVC |
| | | before F38 at | IRKNWNSSDYLHLNFDNFAIQLEKGGKFTVRGKPT |
| | | the N-terminus, | LEDLEQFSEKFYCSCYSTLSCKEKADVKDTDAVDV |
| | | and truncation | CIADGVCIDAFLKPPMETEEPCIFYNASPST |
| | | after T488 at | |
| | | the C-terminus | |
| HM53 | 106 | 15 amino acids | LNFRAPFVIPNVPFLWAWNAPSEFCLGKFDEPLDM |
| | | substitution of | SLFSFIGSPRINATGQGVTIFYVDRLGYYPYIDSI |
| | | T341G, L342W, | TGVTVNGGIPQKISLQDHLDKAKKDITFYMPVDNL |
| | | S343E, I344N, | GMAVIDWEEWRPTWARNWKPKDVYKNRSIELVQQQ |
| | | M345T, S347T, | NVQLSLTEATEKAKQEFEKAGKDFLVSTIKLGKLL |
| | | M348K, K349E, | RPNHLWGYYLFPDCYNHHYKKPGYNGSCFNVEIKR |
| | | L352Q, L353A, | NDDLSMLWNESTALYPSIYLNTQQSPVSATLYVRN |
| | | L354I, D355K, | RVREAIRVSKIPDAKSPLPVFAYTRIVFTDQVLKF |
| | | N356E, E359D | LSQDKLVYTFGETVALGASGIVIWGGWENTRTKES |
| | | and I361T, | CQAIKEYMDTTLNPYIINVTLAAKMCSQVLCQEQG |
| | | truncation | VCIRKMWMSSDYLHLMPDNFAIQLEKGGKFTVRGK |
| | | before L36 at | PTLEDLEQFSEKFYCSCYSTLSCKEKADVKDTDAV |
| | | the N-terminus, | DVCIADGVCIDAFLKPPMETEEPQIFYNASPSTLS |
| | | and truncation | |
| | | after S490 at | |
| | | the C-terminus | |
| HM54 | 107 | 15 amino acids | LNFRAPPVIPNVPFLWAWNAPSEFCLGKFDEPLDM |
| | | substitution of | SLFSFIGSPRINATGQGVTIFYVDRLGYYPYIDSI |
| | | T341A, L342W, | TGVTVNGGIPQKISLQDHLDKAKKDITFYMPVDNL |
| | | S343E, I344N, | GMAVIDWEEWRPTWARNWKPKDVYKNRSIELVQQQ |
| | | M345T, S347T, | NVQLSLTEATEKAKQEFEKAGKDFLVSTIKLGKLL |
| | | M348K, K349E, | RPNHLWGYYLFPDCYNHHYKKPGYNGSCFNVEIKR |
| | | L352Q, L353A, | NDDLSWLWNESTALYPSIYLNTQQSPVAATLYVRK |
| | | L354I, D355K, | RVREAIRVSKIPDAKSPLPVFAYTRIVFTDQVLKF |
| | | N356E, E359D | LSQDELVYTFGETVALGASGIVIWGAWENTRTKES |
| | | and I361T, | CQAIKEYMDTTLNPYIINVTLAAKMCSQVLCQEQG |
| | | truncation | VCIRKNWNSSDYLHLNPDNFAIQLEKGGKFTVRGK |
| | | before L36 at | PTLEDLEQFSEKFYCSCYSTLSCKSKADVKDTDAV |
| | | the N-terminus, | DVCIADGVCIDAFLKPPMETEEPQIFYNASPSTLS |
| | | and truncation | |
| | | after S490 at | |
| | | the C-terminus | |
| HM55 | 108 | 15 amino acids | LNFRAPPVIPNVPFLWAWNAPSEFCLGKFDEPLDM |
| | | substitution of | SLPSFIGSPRINATGQGVTIFYVDRLGYYPYIDSI |
| | | T341C, L342W, | TGVTVNGGIPQKISLQDHLDKAKKDITFYMPVDNL |
| | | S343E, I344N, | GMAVIDWEEWRPTWARNWKPKDVYKNRSIELVQQQ |
| | | M345T, S347T, | NVQLSLTEATEKAKQEFEKAGKDFLVETIKLGKLL |
| | | M348K, K349E, | RPNHLWGYYLFPDCYNHHYKKPGYNGSCFNVEIKR |
| | | L352Q, L353A, | NDDLSWLWNESTALYPSIYLNTQQSPVAATLYVRN |
| | | L354I, D355K, | RVREAIRVSKIPDAKSPLPVFAYTRIVFTDQVLKF |
| | | N356E, E359D | LSQDELVYTFGETVALGASGIVIWGCWENTRTKES |
| | | and I361T, | CQAIKEYMDTTLNPYIINVTLAAKMCSQVLCQEQG |
| | | truncation | VCIRKNWNSSDYLHLNPDNFAIQLEKGGKFTVRGK |
| | | before L36 at | PTLEDLEQFSEKFYCSCYSTLSCKEKADVKDTDAV |
| | | the N-terminus, | DVCIADGVCIDAFLKPPMETEEPQ109IFYNASPS |
| | | and truncation | TLS |
| | | after S490 at | |
| | | the C-terminus | |
| HM56 | 109 | 15 amino acids | LNFRAPPVIPNVPFLWAWNAPSEFCLGKFDEPLDM |
| | | substitution of | SLFSFIGSPRINATGQGVTIFYVDRLGYYPYIDSI |
| | | T341D, L342W, | TGVTVNGGIPQKISLQDHLDKAKKDITFYMPVDNL |
| | | S343E, I344N, | GMAVIDWEEWRPTWARNWKPKDVYKNRSIELVQQQ |
| | | M345T, S347T, | NVQLSLTEATEKAKQEFEKAGKDFLVETIKLGKLL |
| | | M348K, K349E, | RPNRLWGYYLFPDCYNHHYKKPGYNGSCFNVEIKR |
| | | L352Q, L353A, | NDDLSWLWNESTALYPSIYLNTQQSPVAATLYVRN |
| | | L354I, D355K, | RVREAIRVSKIPDAKSPLPVFAYTRIVFTDQVLKF |
| | | N356E, E359D | LSQDELVYTFGETVALCASGIVIWGDWENTRTKES |
| | | and I361T, | CQAIKEYMDTTLNPYIINVTLAAKMCSQVLCQEQG |
| | | truncation | VCIRKNWNSSDYLHLNPDNFAIQLEKGGKFTVRGK |
| | | before L36 at | PTLEDLEQFSEKFYCSCYSTLSCKEKADVKDTDAV |
| | | the N-terminus, | DVCIADGVCIDAFLKPPMETEEFQIFYNASPSTLS |
| | | and truncation | |
| | | after S490 at | |
| | | the C-terminus | |
| HP57 | 110 | 12 amino acids | FRAPPVIPNVPFLWAWNAPSEFCLGKFDEPLDMSL |
| | | substitution of | FSFIGSPRINATGQGVTIFYVDRLGYYPYIDSITG |
| | | I344N, M345T, | VTVNGGIPQKISLQDHLDKAKKDITFYMPVDNLGM |
| | | S347T, M348K, | AVIDWEEWRPTWARNWKPKDVYKNRSIELVQQQNV |
| | | K349E, L352Q, | QLSLTEATEKAKQEFEKAGKDFLVETIKLGKLLRP |
| | | L353A, L354I, | NHLWGYYLFPDCYNHHYKKPGYNGSCFNVEIKRND |
| | | D355K, N356E, | DLSWLWNESTALYPSIYLNTQQSPVAATLYVRNEV |
| | | E359D and I361T, | REAIRVSKIPDAKSPLPVFAYTRIVFTDQVLKFLS |
| | | truncation | QDELVYTFGETVALGASGIVIWGTLSNTRTKESCQ |
| | | before F38 at | AIKEYMDTTLNPYIINVTLAAKMCSQVLCQEQGVC |
| | | the N-terminus, | IRKNWNSSDYLHLNPDNFAIQLSKGGKFTVRGKPT |
| | | and truncation | LEDLEQFSEKFYCSCYSTLSCKEKADVKDTDAVDV |
| | | after F468 at | CIADGVCIDAF |
| | | the C-terminus | |
| HP58 | 111 | 13 amino acids | FRAPPVIPNVPFLWAWNAPSEFCLGKFDEPLDMSL |
| | | substitution of | FSFIGSPRINATGQGVTIFYVDRLGYYPYIDSITG |
| | | S343E, I344N, | VTVNGGIPQKISLQDHLDKAKKDITFYMPVDNLGM |
| | | M345T, S347T, | AVIDWEEWRPTWARNWKPKDVYKNRSIELVQQQNV |
| | | M348K, K349E, | QLSLTEATEKAKQEFEKAGKDFLVETIKLGKLLRP |
| | | L352Q, L353A, | NHLWGYYLFPDCYNHHYKKPGYNGSCFNVEIKRND |
| | | L354I, D355K, | DLSWLWNESTALYPSIYLNTQQSPVAATLYVRNRV |
| | | N356E, E359D | REAIRVSKIPDAKSPLPVFAYTRIVFTDQVLKFLS |
| | | and I361T, | QDELVYTFGETVALGASGIVIWGTLENTRTKESCQ |
| | | truncation | AIKEYMDTTLNPYIINVTLAAKMCSQVLCQEQGVC |
| | | before F38 at | IRKNWNSSDYLHLNPDNFAIQLEKGGKFTVRGKPT |
| | | the N- terminus, | LEDLEQFSEKFYCSCYSTLSCKEKADVKDTDAVDV |
| | | and truncation | CTADGVCIDAF |
| | | after F46S at | |
| | | the C-terminus | |
| HP59 | 112 | 15 amino acids | FRAPPVIPNVPFLWAWNAPSEFCLGKFDEPLDMSL |
| | | substitution of | FSFIGSPRINATGQGVTIFYVDRLGYYPYIDSITG |
| | | T341A, L342W, | VTVNGGIPQKISLQDHLDKAKKDITFYMPVDNLGM |
| | | S343E, I344N, | AVIDWEEWRPTWARNWKPKDVYKKRSIELVQQQNV |
| | | M345T, S347T, | QLSLTEATSKAKQEFEKAGKDFLVETIKLGKLLRP |
| | | M348K, K349E, | NHLWGYYLFPDCYNHHYKKPGYNGSCFNVEIKRND |
| | | L352Q, L353A, | DLSWLWNESTALYPSIYLNTQQSPVAATLYVRNRV |
| | | L354I, D355K, | REAIRVSKIPDAKSPLPVFAYTRIVFTDQVLKFLS |
| | | N356E, E359D | QDELVYTFGETVALGASGIVIWGAWENTRTKESCQ |
| | | and I361T, | AIKEYMDTTLNPYIINVTLAAKMCSQVLGQEQGVC |
| | | truncation | IRKNWNSSDYLHLNPDNFAIQLSKGGKFTVRGKPT |
| | | before F38 at | LEDLEQFSEKFYCSCYSTLSCKSKADVKDTDAVDV |
| | | the N-terminus, | CIADGVCIDAF |
| | | and truncation | |
| | | after F468 at | |
| | | the C-terminus | |
| HP60 | 113 | 15 amino acids | FRAPPVIPNVPFLWAWNAPSEFCLGKFDEPLDMSL |
| | | substitution of | FSFIGSPRINATGQGVTIFYVDRLGYYPYIDSITG |
| | | T341G, L342W, | VTVNGGIPQKISLQDHLDKAKKDITFYMPVDNLGM |
| | | S343E, I344N, | AVIDWEEWRPTWARNWKPKDVYKNRSIELVQQQNV |
| | | M345T, S347T, | QLSLTEATEKAKQEFEKAGKDFLVETIKLGKLLRP |
| | | M348K, K349E, | NHLWGYYLFPDCYNHHYKKPGYNGSCFNVEIKRND |
| | | L352Q, L353A, | DLSWLWNESTALYPSIYLNTQQSPVAATLYVRNRV |
| | | L354I, D355K, | REAIRVSKIPDAKSPLPVFAYTRIVFTDQYLKFLS |
| | | N356E, E359D | QDELVYTFGETVALGASGIVIWGGWENTRTKESCQ |
| | | and I361T, | AIKEYMDTTLNPYIINVTLAAKMCSQVLCQEQGVC |
| | | truncation | IRKNWNSSDYLHLNPDNFAIQLEKGGKFTVRGKPT |
| | | before F38 at | LEDLEQFSEKFYCSCYSTLSCKEKADVKDTDAVDV |
| | | the N-terminus, | CIADGVCIDAF |
| | | and truncation | |
| | | after F468 at | |
| | | the C-terminus | |
| HP61 | 114 | 16 amino acids | FRGPPVIPNVPFLWAWNAPSEFCLGKFDEPLDMSL |
| | | substitution of | FSFIGSPRINATGQGVTIFYVDRLGYYPYIDSITG |
| | | A40G, T341S, | VTVNGGIPQKISLQDHLDKAKKDITFYMPVDNLGM |
| | | L342W, S343E, | AVIDWEEWRPTWARNWKPKDVYKKRSIELVQQQNV |
| | | 1344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTFGETVALGASGIVIWGSWENTRTKESCQ |
| | | E359D and I361T, | AIKEYMDTTLNPYIINVTLAAKMCSQVLCQEQGVC |
| | | truncation | IRKNWNSSDYLHLNPDNFAIQLEKGGKFTVRGKPT |
| | | before F38 at | LEDLEQFSEKFYCSCYSTLSCKEKADVKDTDAVDV |
| | | the N-terminus, | CIADGVCIDAF |
| | | and truncation | |
| | | after F468 at | |
| | | the C-terminus | |
| HP62 | 115 | Removal of P42, | FRGPPVIPNVPFLWAWNAPSEFCLGKFDEPLDMSL |
| | | 15 amino acids | FSFIGSPRINATGQGVTIFYVDRLGYYPYIDSITG |
| | | substitution of | VTVNGGIPQKISLQDHLDKAKKDITFYMPVDHLGM |
| | | T341S, L342W, | AVIDWEEWRPTWARNWKPKDVYKNRSIELVQQQNV |
| | | S343E, 1344N, | QLSLTEATEKAKQEFEKAGKDELVETIKLGKLLRP |
| | | M345T, S347T, | HHLWGYYLFPDCYNHHYKKPGYNGSCFNVEIKRND |
| | | M348K, K349E, | DLSWLWNESTALYPSIYLNTQQSPVAATLYVRNRV |
| | | L352Q, L353A, | REAIRVSKIPDAKSPLPVFAYTRIVFTDQVLKFLS |
| | | L354I, D355K, | QDELVYTFGETVALGASGIVIWGSWENTRTKESCQ |
| | | N356E, E359D | AIKEYMDTTLNPYIINVTLAAKMCSQVLCQEQGVC |
| | | and I361T, | IRKNWNSSDYLHLNPDNFAIQLEKGGKFTVRGKPT |
| | | truncation | LEDLEQFSEKFYCSCYSTLSCKEKADVKDTDAVDV |
| | | before F38 at | CIADGVCIDAF |
| | | the N-terminus, | |
| | | and truncation | |
| | | after F468 at | |
| | | the C-terminus | |

In some embodiments the variant or fragment of hyaluronidase is a hyaluronidase enzyme where the N-terminus further includes a human growth hormone signal peptide having an amino acid sequence MATGSRTSLLLAFGLLCLPWLQEGSA of SEQ ID NO: 3, a human serum albumin signal peptide having an amino acid sequence MKWVTFISLLFLFSSAYS of SEQ ID NO: 4, or a human Hyal1 signal peptide having an amino acid sequence MAAHLLPICALFLTLLDMAQG of SEQ ID NO: 5 as shown in Table 5 below, instead of the signal peptide of wild-type PH20, which consists of M1 to T35, but is not limited thereto. In embodiments, the signal peptide of wild-type PH20 (amino acid residues M1 to T35) is partially or completely deleted. In some embodiments, where a portion of the N-terminus is further deleted, for example, a case in which cleavage occurs before the N37, F38, R39, A40, P41 or P42 residue occurs so that an additional deletion of the N-terminus together with the deletion of the signal peptide of wild-type PH20 occurs.

TABLE 5
Amino acid sequences of signal peptide
of human growth hormone, human serum
albumin or human Hyal1

| | Amino acid | SEQ ID |
|---|---|---|
| | Sequence | NO. |
| Human growth | MATGSRTSLLLAF | 3 |
| hormone | GLLCLPWLQEGSA | |
| Human serum | MKWVTFISLLFLF | 4 |
| albumin | SSAYS | |
| Human HYAL1 | MAAHLLPICALFL | 5 |
| | TLLDMAQG | |

In some embodiments, the variant or fragment of hyaluronidase includes a variant having a C-terminal to which a 6×His-tag is attached (denoted as HM), a variant without a 6×His-tag (denoted as HP), a mature wild-type PH20 (L36-S490) having a C-terminal to which a 6×His-tag is attached (denoted as WT), and mature wild-type PH20 (L36 to Y482) that does not contain a 6×His-tag and has a C-terminal in which cleavage occurs after Y482 (denoted as HW2).

In certain embodiments, compositions include one or more variant or fragment of a hyaluronidase enzyme, such those described in European Patent Publication No. EP3636752A1 and International Patent Publication No. WO 2020/197230, the disclosures of which are herein incorporated by reference.

In embodiments, the amount of hyaluronidase enzyme present in the composition may vary and may be 100 U or more, such as 250 U or more, such as 500 U or more, such as 750 U or more, such as 1000 U or more, such as 1500 U or more, such as 2000 U or more, such as 2500 U or more, such as 3000 U or more, such as 3500 U or more, such as 4000 U or more, such as 4500 U or more, such as 5000 U or more, such as 10,000 U or more, such as 20,000 U or more, such as 30,000 U or more, such as 40,000 U or more and including 50,000 U of hyaluronidase enzyme or more. In some embodiments, the composition includes about 500 U of the hyaluronidase enzyme, about 600 U, about 700 U, about 800 U, about 900 U, about 1,000 U, about 1,100 U, about 1,200 U, about 1,300 U, about 1,400 U, about 1,500 U, about 1,600 U, about 1,700 U, about 1,800 U, about 1,900 U, about 2,000 U, about 2,100 U, about 2,200 U, about 2,300 U, about 2,400 U, about 2,500 U, about 2,600 U, about 2,700 U, about 2,800 U, about 2,900 U, about 3,000 U, about 3,100 U, about 3,200 U, about 3,300 U, about 3,400 U, about 3,500 U, about 3,600 U, about 3,700 U, about 3,800 U, about 3,900 U, about 4,000 U, about 4,100 U, about 4,200 U, about 4,300 U, about 4,400 U, about 4,500 U, about 4,600 U, about 4,700 U, about 4,800 U, about 4,900 U or about 5,000 U of the hyaluronidase enzyme. For example, the amount of hyaluronidase enzyme in the composition may range from 50 U to 50,000 U, such as from 100 U to 45,000 U, such as from 250 U to 40,000 U, such as from 500 U to 35,000 U, such as from 750 U to 30,000 U, such as from 1000 U to 25,000 U, such as from 1500 U to 20,000 U, such as from 2000 U to 15,000 U, such as from 2500 U to 10,000 U and including from 3000 U to 5000 U of the hyaluronidase enzyme. For example, the amount of hyaluronidase enzyme in the composition may range from 50 U to 50,000 U, such as from 100 U to 40,000 U, such as from 300 U to 30,000 U, such as from 500 U to 20,000 U, such as from 700 U to 10,000 U, such as from 800 U to 5,000 U, such as from 900 U to 4,000 U, such as from 1,000 U to 3,000 U, such as from 1,500 U to 2,500 U and including from 1,700 U to 2,200 U of the hyaluronidase enzyme.

In certain embodiments, the concentration of the hyaluronidase enzyme in the composition is 50 U/mL or more, such as 100 U/mL or more, such as 250 U/mL or more, such as 500 U/mL or more, such as 750 U/mL or more, such as 1000 U/mL or more, such as 2000 U/mL or more, such as 2500 U/mL or more, such as 3000 U/mL or more, such as 3500 U/mL or more, such as 4000 U/mL or more, such as 4500 U/mL or more, such as 5000 U/mL or more. In some instances, the concentration of the hyaluronidase enzyme is about 100 U/mL, 200 U/mL, 300 U/mL, 400 U/mL 500 U/mL, about 600 U/mL, about 700 U/mL, about 800 U/mL, about 900 U/mL, about 1,000 U/mL, about 1,100 U/mL, about 1,200 U/mL, about 1,300 U/mL, about 1,400 U/mL, about 1,500 U/mL, about 1,600 U/mL, about 1,700 U/mL, about 1,800 U/mL, about 1,900 U/mL, about 2,000 U/mL, about 2,100 U/mL, about 2,200 U/mL, about 2,300 U/mL, about 2,400 U/mL, about 2,500 U/mL, about 2,600 U/mL, about 2,700 U/mL, about 2,800 U/mL, about 2,900 U/mL, about 3,000 U/mL, about 3,100 U/mL, about 3,200 U/mL, about 3,300 U/mL, about 3,400 U/mL, about 3,500 U/mL, about 3,600 U/mL, about 3,700 U/mL, about 3,800 U/mL, about 3,900 U/mL, about 4,000 U/mL, about 4,100 U/mL, about 4,200 U/mL, about 4,300 U/mL, about 4,400 U/mL, about 4,500 U/mL, about 4,600 U/mL, about 4,700 U/mL, about 4,800 U/mL, about 4,900 U/mL or about 5,000 U/mL. For example, the concentration of the hyaluronidase enzyme in the composition may range from 50 U/mL to 5000 U/mL, such as from 100 U/mL to 4500 U/mL, such as from 250 U/mL to 4000 U/mL, such as from 500 U/mL to 3500 U/mL, such as from 750 U/mL to 3000 U/mL, including from 1000 U/ml to 2000 U/ml and including from 1500 U/mL to 2500 U/mL.

Compositions according to embodiments also include a telomerase inhibitor having an oligonucleotide and a lipid moiety linked to the 5′ and/or 3′ end of the oligonucleotide. In embodiments, the term telomerase inhibitor as used herein refers to a compound which is capable of reducing or inhibiting the activity of telomerase reverse transcriptase enzyme in a mammalian cell. Telomerase inhibitors of interest, in some instances, include a hTR template inhibitor including an oligonucleotide. An “hTR template inhibitor” is a compound that blocks the template region of the RNA component of human telomerase and can inhibit the activity of the enzyme. In some embodiments, the oligonucleotide includes a sequence effective to hybridize to a more specific portion of this region, having sequence 5′-CUAACCCUAAC-3′.

Telomerase inhibitors of interest include an oligonucleotide and a lipid moiety linked to the 5′ and/or 3′ end of the oligonucleotide. In some embodiments, the telomerase inhibitor includes an oligonucleotide having “nuclease-resistant linkages” having a backbone with subunit linkages that are substantially resistant to nuclease cleavage, in non-hybridized or hybridized form, by extracellular and intracellular nucleases. In some instances, the oligonucleotide shows little or no nuclease cleavage under physiological conditions.

The region of the therapeutic oligonucleotide that is targeted to the hTR sequence is, in some embodiments, complementary to the corresponding hTR sequence. In certain embodiments, the base sequence of the oligonucleotide includes a sequence of 5 nucleotides or more that are complementary to the hTR target, such as 8 nucleotides or more, such as 10 nucleotides or more, such as 12 nucleotides or more, such as 15 nucleotides or more that are complementary to the hTR target. In certain embodiments, oligonucleotides in telomerase inhibitors of the present disclosure are fully complimentary to the hTR target sequence, such as where the full length of the oligonucleotide is complementary to the hTR target sequence.

The telomerase inhibitor includes internucleoside linkages, such as phosphodiester, phosphotriester, methylphosphonate, P3′→N5′ phosphoramidate, N3′→P5′ phosphoramidate, N3′→P5′ thiophosphoramidate, and phosphorothioate linkages. In certain embodiments, telomerase inhibitors of interest include at least one N3′→P5′ phosphoramidate (NP) or N3′→P5′ thiophosphoramidate (NPS) linkage, which linkage may be represented by the structure: 3′-(—NH—P(═O)(—XR)—O—)-5′, wherein X is O or S and R is selected from the group consisting of hydrogen, alkyl, and aryl; and pharmaceutically acceptable salts thereof, when XR is OH or SH. In other embodiments, the oligonucleotide includes all NP or, in some embodiments, all NPS linkages. In one embodiment, the sequence for an hTR template inhibitor oligonucleotide is the sequence complementary to nucleotides 42-54 of SEQ ID NO: 6 (GGGUUGCGGAGGGUGGGCCUGGGAGGGGUGGUGGCCAUUU UUUGUCUAACCCUAACUGAGAAGGGCGUAGGCGCCGUGCUUUUGCUCCCC GCGCGCUGUUUUUCUCGCUGACUUUCAGCGGGCGGAAAAGCCUCGGCCUG CCGCCUUCCACCGUUCAUUCUAGAGCAAACAAAAAAUGUCAGCUGCUGGC CCGUUCGCCUCCCGGGGACCUGCGGCGGGUCGCCUGCCCAGCCCCCGAAC CCCGCCUGGAGCCGCGGUCGGCCCGGGGCUUCUCCGGAGGCACCCACUGC CACCGCGAAGAGUUGGGCUCUGUCAGCCGCGGGUCUCUCGGGGGCGAGGG CGAGGUUCACCGUUUCAGGCCGCAGGAAGAGGAACGGAGCGAGUCCCGCC GCGGCGCGAUUCCCUGAGCUGUGGGACGUGCACCCAGGACUCGGCUCACA CAUGCAGUUCGCUUUCCUGUUGGUGGGGGGAACGCCGAUCGUGCGCAUCC GUCACCCCUCGCCGGCAGUGGGGGCUUGUGAACCCCCAAACCUGACUGAC UGGGCCAGUGUGCU). In certain embodiments, the oligonucleotide includes a sequence which is complementary or near-complementary to some portion of the 11-nucleotide region having the sequence 5′-CUAACCCUAAC-3′ The oligonucleotide having this sequence (TAGGGTTAGACAA; SEQ ID NO:17) and N3′→P5′ thiophosphoramidate (NPS) linkages is designated herein as GRN163. See, for example, Asai et al., Cancer Research 63:3931-3939 (2003) and Gryaznov et al., Nucleosides Nucleotides Nucleic Acids 22(5-8):577-81 (2003). Another target region is the region spanning nucleotides 137-179 of hTR (see Pruzan et al., Nucl. Acids Research, 30:559-568, 2002). Within this region, the sequence spanning 141-153 is a preferred target. PCT publication WO 98/28442 describes the use of oligonucleotides of at least 7 nucleotides in length to inhibit telomerase, where the oligonucleotides are designed to be complementary to accessible portions of the hTR sequence outside of the template region, including nucleotides 137-196, 290-319, and 350-380 of hTR. Preferred hTR targeting sequence are given below, and identified by SEQ ID NOS: 7-27. In certain embodiments, the oligonucleotide of the telomerase inhibitor has a sequence targeting human telomerase RNA (hTR), including but not limited to the sequences:

| | Region of | |
|---|---|---|
| hTR Targeting | SEQ ID | SEQ ID |
| Sequence | NO: 6 | NO: |
| ACATTTTTTGTTTGCTCTAG | 160-179 | 7 |
| GCTCTAGAATGAACGGTGGA | 137-166 | 8 |
| AGGCGGCAGG | | |
| GTGGAGGCGGCAGG | 137-151 | 9 |
| GGAAGGCGGCAGG | 137-149 | 10 |
| GTGGAAGGCGGCA | 139-151 | 11 |
| GTGGAAGGCGG | 141-151 | 12 |
| CGGTGGAAGGCGG | 141-153 | 13 |
| ACGGTGGAAGGCG | 142-154 | 14 |
| AACGGTGGAAGGCGGC | 143-155 | 15 |
| ATGAACGGTGGAAGGCGG | 144-158 | 16 |
| TAGGGTTAGACAA | 42-54 | 17 |
| CAGTTAGGGTTAG | 46-58 | 18 |
| TAGGGTTAGACA | 42-53 | 19 |
| TAGGGTTAGAC | 42-52 | 20 |
| GTTAGGGTTAG | 46-56 | 21 |
| GTTAGGGTTAGAC | 44-56 | 22 |
| GTTAGGGTTAGACAA | 42-56 | 23 |
| GGGTTAGAC | 44-52 | 24 |
| CAGTTAGGG | 50-58 | 25 |
| CCCTTCTCAGTT | 54-65 | 26 |
| CGCCCTTCTCAG | 56-67 | 27 |

Telomerase inhibitors of the present disclosure include a lipid moiety linked to the 5′ and/or 3′ end of the oligonucleotide. In some instances, structural group provides for superior cellular uptake properties, such that an equivalent biological effect may be obtained using smaller amounts of the conjugated oligonucleotide compared to the unmodified form. The lipid moiety may be an aliphatic hydrocarbon or fatty acid, such as derivatives of hydrocarbons and fatty acids. For instance, the lipid moiety may be saturated straight chain compounds having 14-20 carbons, such as myristic (tetradecanoic) acid, palmitic (hexadecanoic) acid, and stearic (octadeacanoic) acid, and their corresponding aliphatic hydrocarbon forms, tetradecane, hexadecane and octadecane. Examples of other lipid moieties include sterols, such as cholesterol, and substituted fatty acids and hydrocarbons, particularly polyfluorinated forms of these groups. In certain embodiments, the lipid moiety includes one or more derivatives such as amine, amide, ester and carbamate derivative of the lipid moiety. In one example, the lipid moiety is a palmitoyl (C16) moiety, such as palmitoyl amide. The lipid moiety may be conjugated to the oligonucleotide through a linker, such as a glycerol or aminoglycerol linker.

In some embodiments, the telomerase inhibitor is a compound as described in U.S. Pat. No. 9,375,485, the disclosure of which is herein incorporated by reference. In certain embodiments, the telomerase inhibitor is imetelstat (5′ palmitoylated 13-mer thiophosphoramidate oligonucleotide composed of the sequence 5′-TAGGGTTAGACAA-3′ SEQ ID NO:17) or a pharmaceutically acceptable salt thereof, such as imetelstat sodium:

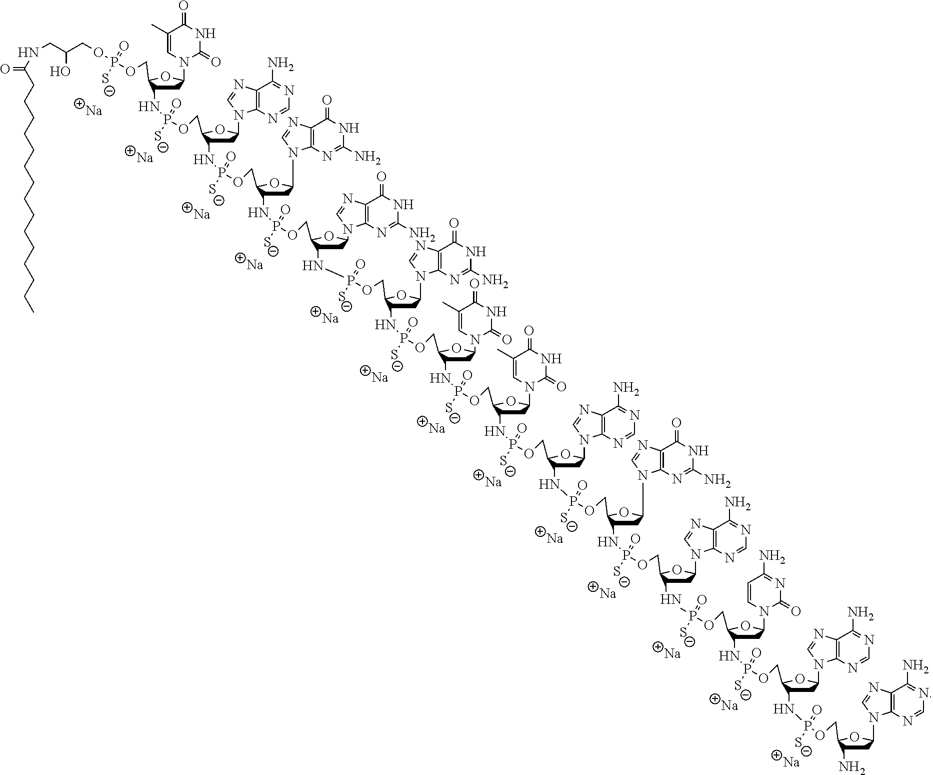

In some embodiments, the amount of telomerase inhibitor, such as imetelstat or imetelstat sodium, in the subcutaneous composition is from about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg, about 500 mg to about 600 mg, about 600 mg to about 700 mg, about 700 mg to about 800 mg, about 800 mg to about 900 mg, about 900 mg to about 1000 mg, about 1100 mg to about 1200 mg, about 1200 mg to about 1300 mg, about 1300 mg to about 1400 mg, about 1400 mg to about 1500 mg, about 1500 mg to about 1600 mg, about 1600 mg to about 1700 mg, about 1700 mg to about 1800 mg, about 1800 mg to about 1900 mg, about 1900 mg to about 2000 mg, about 2000 mg to about 2100 mg, about 2100 mg to about 2200 mg, about 2200 mg to about 2300 mg, about 2300 mg to about 2400 mg, about 2400 mg to about 2500 mg.

In some embodiments, the amount of a telomerase inhibitor is in a unit dosage form having an amount in the range of from about 5 mg to about 1000 mg, 5 mg to about 500 mg, such as about 30 mg to about 300 mg or about 50 mg to about 200 mg. In some embodiments, the amount of a telomerase inhibitor is in a unit dosage form having an amount in the range of from about 200 mg to about 3000 mg, 750 mg to about 2500 mg, such as about 1000 mg to about 2000 mg or about 500 mg to about 2000 mg. The unit dosage form may be liquid or lyophilized.

In some embodiments, the concentration of the telomerase inhibitor in the composition is dilute (about 0.1 mg/ml) or concentrated (about 300 mg/ml), including for example any of about 0.1 to about 300 mg/ml, 0.1 to about 200 mg/ml, about 0.1 to about 180 mg/ml, about 0.1 to about 160 mg/ml, about 0.1 to about 140 mg/ml, about 0.1 to about 120 mg/ml, about 0.1 to about 100 mg/ml, about 0.1 to about 80 mg/ml, about 0.1 to about 60 mg/ml, about 0.1 to about 40 mg/ml, about 0.1 to about 20 mg/ml, about 0.1 to about 10 mg/ml about 2 to about 40 mg/ml, about 4 to about 35 mg/ml, about 6 to about 30 mg/ml, about 8 to about 25 mg/ml, about 10 to about 20 mg/ml, about 12 to about 15 mg/ml, or any of about 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2 mg/ml, 2.1 mg/ml, 2.2 mg/ml, 2.3 mg/ml, 2.4 mg/ml, or 2.5 mg/ml. In some embodiments, the concentration of the telomerase inhibitor is at least about any of 0.1 mg/ml, 0.2 mg/ml, 0.3 mg/ml, 0.4 mg/ml, 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 33.3 mg/ml, 34 mg/ml, 35 mg/ml, 36 mg/ml, 37 mg/ml, 38 mg/ml, 39 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml, 200 mg/ml, 210 mg/ml, 220 mg/ml, 230 mg/ml, 240 mg/ml, or 250 mg/ml, 260 mg/ml, 270 mg/ml, 280 mg/ml, 290 mg/ml, 300 mg/ml.

In certain embodiments, the composition is formulated to include the telomerase inhibitor, such as imetelstat or imetelstat sodium, at a dosage ranging from about 2.0 mg/kg to about 20.0 mg/kg, such as from about 3.0 mg/kg to about 15.0 mg/kg, such as from about 4.0 mg/kg to about 10 mg/kg, such as from about 6 mg/kg to about 14 mg/kg, such as from about 7 mg/kg to about 13 mg/kg, such as from about 8 mg/kg to about 12 mg/kg, such as from about 7.5 mg/kg to 9.4 mg/kg, including from about 9 mg/kg to about 11 mg/kg, and including from about 11 mg/kg to about 14 mg/kg. In some embodiments, the composition is formulated to include the telomerase inhibitor, such as imetelstat or imetelstat sodium, at dosage ranging from about 7.5 mg/kg to about 9.4 mg/kg. For example, the dosage of telomerase inhibitor may be 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10 mg/kg, 10.1 mg/kg, 10.2 mg/kg, 10.3 mg/kg, 10.4 mg/kg, 10.5 mg/kg, 10.6 mg/kg, 10.7 mg/kg, 10.8 mg/kg, 10.9 mg/kg, 11 mg/kg, 11.1 mg/kg, 11.2 mg/kg, 11.3 mg/kg, 11.4 mg/kg, 11.5 mg/kg, 11.6 mg/kg, 11.7 mg/kg, 11.8 mg/kg, 11.9 mg/kg, 12 mg/kg, 12.1 mg/kg, 12.2 mg/kg, 12.3 mg/kg, 12.4 mg/kg, 12.5 mg/kg, 12.6 mg/kg, 12.7 mg/kg, 12.8 mg/kg, 12.9 mg/kg, 13 mg/kg, 10.5 mg/kg, 11.0 mg/kg, 11.5 mg/kg, 12.0 mg/kg, 12.5 mg/kg, 13.0 mg/kg, 13.5 mg/kg, 14.0 mg/kg, 14.5 mg/kg, 15.0 mg/kg, 15.5 mg/kg, 16.0 mg/kg, 16.5 mg/kg, 17.0 mg/kg, 17.0 mg/kg, 17.5 mg/kg, 18.0 mg/kg, 18.5 mg/kg, 19.0 mg/kg, 19.5 mg/kg, or 20.0 mg/kg.

In some embodiments, the subcutaneous telomerase inhibitor composition also includes one or more pharmaceutically acceptable carriers. Exemplary pharmaceutically acceptable carriers may include solvents, dispersion media, coatings, antibacterial and antifungal agents, isotonic and absorption delaying agents, aqueous or non-aqueous carriers, or combinations thereof. In certain embodiments, compositions may further include one or more pharmaceutically acceptable excipients as part of a pharmaceutical composition. Excipients may include, but are not limited to, carbohydrates, inorganic salts, antimicrobial agents, stabilizing agents, antioxidants, surfactants, amino acids, buffers, acids, bases, and combinations thereof. For example, excipients suitable for subcutaneously injectable compositions may include one or more of water, alcohols, polyols, monosaccharides, polysaccharides, stabilizing agents, buffers, amino acids, and surfactants. The amount of each pharmaceutically acceptable excipient or carrier may vary and may range from 1 mM to 1000 mM, such as from 2 mM to 900 mM, such as from 3 mM to 800 mM, such as from 4 mM to 700 mM, such as from 5 mM to 600 mM, such as from 6 mM to 500 mM, such as from 7 mM to 400 mM, such as from 8 mM to 300 mM, such as from 9 mM to 200 mM and including from 10 mM to 100 mM.

In some embodiments, compositions include a buffer. Example buffers that may be used are acetic acid, citric acid, formic acid, succinic acid, phosphoric acid, carbonic acid, malic acid, aspartic acid, histidine, boric acid, Tris buffers, HEPPSO and HEPES. In some instances, the buffers are present in the composition in an amount to maintain the composition at a predetermined pH. For example, the one or more buffers may be present in the composition to maintain the composition at a pH of from 3.0 to 9.0, such as a pH of from 3.5 to 8.5, such as a pH of from 4.0 to 8.0, such as a pH of from 4.5 to 7.5, such as a pH of from 5.0 to 7.0 and including a pH of from 5.5 to 7.5. For example, the composition may have a pH of 3.0, pH of 3.1, pH of 3.2, pH of 3.3, pH of 3.4, pH of 3.5, pH of 3.6, pH of 3.7, pH of 3.8, pH of 3.9, pH of 4.0, pH of 4.1, pH of 4.2, pH of 4.3, pH of 4.4, pH of 4.5, pH of 4.6, pH of 4.7, pH of 4.8, pH of 4.9, pH of 5.0, pH of 5.1, pH of 5.2, pH of 5.3, pH of 5.4, pH of 5.5, pH of 5.6, pH of 5.7, pH of 5.8, pH of 5.9, pH of 6.0, pH of 6.1, pH of 6.2, pH of 6.3, pH of 6.4, pH of 6.5, pH of 6.6, pH of 6.7, pH of 6.8, pH of 6.9, pH of 7.0, pH of 7.1, pH of 7.2, pH of 7.3, pH of 7.4, pH of 7.5, pH of 7.6, pH of 7.7, pH of 7.8, pH of 7.9, pH of 8.0, pH of 8.1, pH of 8.2, pH of 8.3, pH of 8.4, pH of 8.5, pH of 8.6, pH of 8.7, pH of 8.8, pH of 8.9 or a pH of 9.0. The buffer may be present in the composition in an amount of from 1 mM to 1000 mM, such as from 2 mM to 900 mM, such as from 3 mM to 800 mM, such as from 4 mM to 700 mM, such as from 5 mM to 600 mM, such as from 6 mM to 500 mM, such as from 7 mM to 400 mM, such as from 8 mM to 300 mM, such as from 9 mM to 200 mM and including from 10 mM to 100 mM. For example, the buffer may be present in the composition at a concentration of about 10 mM, about 20 mM, about 30 mM, about 40 mM, about 50 mM, about 75 mM, 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM or about 500 mM.

In some embodiments, compositions include a carbohydrate, such as a saccharide. Example saccharides include monosaccharides, di saccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol or iso-maltulose. In some instances, compositions include sucrose. In other instances, compositions include trehalose. The carbohydrate (e.g., saccharide such as sucrose or trehalose) may be present in the composition in an amount of from 1 mM to 1000 mM, such as from 2 mM to 900 mM, such as from 3 mM to 800 mM, such as from 4 mM to 700 mM, such as from 5 mM to 600 mM, such as from 6 mM to 500 mM, such as from 7 mM to 400 mM, such as from 8 mM to 300 mM, such as from 9 mM to 200 mM and including from 10 mM to 100 mM. For example, the carbohydrate (e.g., saccharide such as sucrose or trehalose) may be present in the composition at a concentration of about 10 mM, about 20 mM, about 30 mM, about 40 mM, about 50 mM, about 75 mM, 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM or about 500 mM.

In some embodiments, compositions include one or more amino acids. Example amino acids include histidine, isoleucine, methionine, glycine, arginine, lysine, L-leucine, tri-leucine, alanine, glutamic acid, L-threonine, and 2-phenylamine. In some instances, compositions include methionine. In other instances, compositions include histidine. The amino acid (e.g., methionine or histidine) may be present in the composition in an amount of from 0.1 mg/mL to about 5 mg/mL, such as from 0.1 mg/mL to about 2.5 mg/mL, such as from 1 mg/mL to about 2 mg/mL, such as from 4 mM to 700 mM, such as from 5 mM to 600 mM, such as from 6 mM to 500 mM, such as from 7 mM to 400 mM, such as from 8 mM to 300 mM, such as from 9 mM to 200 mM and including from 10 mM to 100 mM. For example, the amino acid (e.g., methionine or histidine) may be present in the composition at a concentration of about 0.5 mg/mL, about 1 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, about 3 mg/mL, about 3.5 mg/mL, about 4 mg/mL, about 4.5 mg/mL or about 5 mg/mL. In certain embodiments, compositions include histidine in an amount of about 0.5 mg/mL, about 1 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, about 3 mg/mL, about 3.5 mg/mL, about 4 mg/mL, about 4.5 mg/mL or about 5 mg/mL. In other embodiments, compositions include methionine in an amount of about 0.5 mg/mL, about 1 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, about 3 mg/mL, about 3.5 mg/mL, about 4 mg/mL, about 4.5 mg/mL or about 5 mg/mL.

In some embodiments, compositions include one or more surfactants. Example surfactants include polysorbates (e.g., polysorbate-20 or polysorbate-80); polyoxamers (e.g., poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™, PF68, etc.). In some instances, compositions include a polysorbate surfactant. The surfactant may be present in the composition in an amount of from 1 mM to 1000 mM, such as from 2 mM to 900 mM, such as from 3 mM to 800 mM, such as from 4 mM to 700 mM, such as from 5 mM to 600 mM, such as from 6 mM to 500 mM, such as from 7 mM to 400 mM, such as from 8 mM to 300 mM, such as from 9 mM to 200 mM and including from 10 mM to 100 mM. For example, the surfactant may be present in the composition at a concentration of about 10 mM, about 20 mM, about 30 mM, about 40 mM, about 50 mM, about 75 mM, 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM or about 500 mM.

Compositions may include one or more pharmaceutically acceptable salts. Pharmaceutically acceptable salts may be (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3 (4 hydroxybenzoyl) benzoic acid, cinnamic acid, mandelic acid, methanesulfonic acid, ethanesulfonic acid, 1,2 ethane disulfonic acid, 2 hydroxyethanesulfonic acid, benzenesulfonic acid, 4 chlorobenzenesulfonic acid, 2 naphthalenesulfonic acid, 4 toluenesulfonic acid, camphorsulfonic acid, 4 methylbicyclo[2.2.2] oct 2 ene 1 carboxylic acid, glucoheptonic acid, 3 phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the compound is replaced by a metal ion, e.g., an alkali metal ion, an alkaline earth ion, or an aluminum ion; or coordinates with an organic base such as ethanolamine, diethanolamine, triethanolamine, N methylglucamine and the like. In certain embodiments, compositions include a sodium chloride salt or other pharmaceutically acceptable salt, such as magnesium sulfate. The pharmaceutically acceptable salt may be present in the composition in an amount of from 1 mM to 1000 mM, such as from 2 mM to 900 mM, such as from 3 mM to 800 mM, such as from 4 mM to 700 mM, such as from 5 mM to 600 mM, such as from 6 mM to 500 mM, such as from 7 mM to 400 mM, such as from 8 mM to 300 mM, such as from 9 mM to 200 mM and including from 10 mM to 100 mM. For example, the pharmaceutically acceptable salt may be present in the composition at a concentration of about 10 mM, about 20 mM, about 30 mM, about 40 mM, about 50 mM, about 75 mM, 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM or about 500 mM.

Acids or bases may also be present in the subject compositions. For example, acids may include but are not limited to hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and any combinations thereof. Examples bases include, but are not limited to sodium hydroxide, sodium acetate, ammonium hydroxide, potassium hydroxide, ammonium acetate, potassium acetate, sodium phosphate, potassium phosphate, sodium carbonate monohydrate, sodium citrate, sodium formate, sodium sulfate, potassium sulfate, potassium fumarate, and any combinations thereof. The acid or base may be present in the composition at a concentration of from 1 mM to 1000 mM, such as from 2 mM to 900 mM, such as from 3 mM to 800 mM, such as from 4 mM to 700 mM, such as from 5 mM to 600 mM, such as from 6 mM to 500 mM, such as from 7 mM to 400 mM, such as from 8 mM to 300 mM, such as from 9 mM to 200 mM and including from 10 mM to 100 mM. For example, the acid or base may be present in the composition at a concentration of about 10 mM, about 20 mM, about 30 mM, about 40 mM, about 50 mM, about 75 mM, 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM or about 500 mM.

In some embodiments, compositions include one or more antioxidants. Antioxidants, which can reduce or prevent oxidation and thus deterioration of the composition, may include, for example, ascorbyl palmitate, butylated hydroxyanisole, butylated hydroxytoluene, hypophosphorous acid, monothioglycerol, propyl gallate, sodium bisulfate, sodium formaldehyde sulfoxylate, sodium metabisulfite, and any combinations thereof. The antioxidant may be present in the composition at a concentration of from 1 mM to 1000 mM, such as from 2 mM to 900 mM, such as from 3 mM to 800 mM, such as from 4 mM to 700 mM, such as from 5 mM to 600 mM, such as from 6 mM to 500 mM, such as from 7 mM to 400 mM, such as from 8 mM to 300 mM, such as from 9 mM to 200 mM and including from 10 mM to 100 mM. For example, the antioxidant may be present in the composition at a concentration of about 10 mM, about 20 mM, about 30 mM, about 40 mM, about 50 mM, about 75 mM, 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM or about 500 mM.

In some embodiments, compositions include one or more preservatives. Preservatives, which can reduce or prevent degradation of the composition, such as by microbial growth, may include, for example, antioxidants, antimicrobial agents and chelating agents, and may include methyl, ethyl, propyl and butyl parabens, aryl and alkyl acids, citric acid, sorbic acid, Na, K & Ca sorbate, benzoic acid, Na, K & Ca benzoate, benzyl alcohol, sodium metabisulfite, bronopol, propylene glycol (15-30%), BHT (butylatedhydroxytoluene), BHA (butylatedhydroxyanisole), propyl gallate, EDTA, chlorobutanol, benzaldehyde, phenol, meta cresol, chloro cresol, benzylkonium chloride, benzethonium chloride, and mercury compounds such as thiomersal, phenylmercuric nitrate, and any combinations thereof. The preservative may be present in the composition at a concentration of from 1 mM to 1000 mM, such as from 2 mM to 900 mM, such as from 3 mM to 800 mM, such as from 4 mM to 700 mM, such as from 5 mM to 600 mM, such as from 6 mM to 500 mM, such as from 7 mM to 400 mM, such as from 8 mM to 300 mM, such as from 9 mM to 200 mM and including from 10 mM to 100 mM. For example, the preservative may be present in the composition at a concentration of about 10 mM, about 20 mM, about 30 mM, about 40 mM, about 50 mM, about 75 mM, 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM or about 500 mM.

Pharmaceutical excipients along with other excipients that may be employed in the subject telomerase inhibitor compositions are described in A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc., the disclosure of which is incorporated herein by reference.

### Methods for Subcutaneously Administering a Telomerase Inhibitor Composition

Aspects of the disclosure also include methods for subcutaneously administering a telomerase inhibitor composition to a subject. In practicing methods according to certain embodiments, one or more compositions as described herein having a telomerase inhibitor and a hyaluronidase enzyme is subcutaneously administered to the subject. In some embodiments, the composition is administered to the subject by subcutaneous injection or subcutaneous infusion. In other embodiments, the composition may be administered to the subject from an implanted device, such as a subcutaneously-implanted catheter. In certain embodiments, the telomerase inhibitor composition is administered to the subject with a subcutaneous bolus injector configured to subcutaneously deliver a predetermined amount of the composition to the subject.

In some embodiments, methods include subcutaneously administering one or more compositions as described herein having a telomerase inhibitor and a hyaluronidase enzyme to a subject to treat a neoplasm. In some embodiments, the neoplasm may be a solid-tumor cancer. Examples of cancers for treatment according to embodiments of the present disclosure may include but are not limited to, e.g., Adrenocortical Carcinoma, Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Carcinoid Tumor (e.g., Childhood, Gastrointestinal, etc.), Carcinoma of Unknown Primary, Cardiac (Heart) Tumors, Cervical Cancer, Colon Cancer, Colorectal Cancer, Craniopharyngioma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, etc.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hypopharyngeal Cancer, Intraocular Melanoma, Islet Cell Tumors (e.g., Pancreatic Neuroendocrine Tumors, etc.), Kidney Cancer (e.g., Renal Cell, Wilms Tumor, Childhood Kidney Tumors, etc.), Langerhans Cell Histiocytosis, Laryngeal Cancer, Lip and Oral Cavity Cancer, Liver Cancer (Primary), Lobular Carcinoma In Situ (LCIS), Lung Cancer (e.g., Non-Small Cell, Small Cell, etc.), Malignant Fibrous Histiocytoma of Bone and Osteosarcoma, Melanoma, Merkel Cell Carcinoma, Mesothelioma, Metastatic Squamous Neck Cancer with Occult Primary, Mouth Cancer, Multiple Endocrine Neoplasia Syndromes, Nasal Cavity and Paranasal Sinus Cancer, Nasopharyngeal Cancer, Neuroblastoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sézary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like. In certain embodiments, methods include treating a subject having a neoplasm as described in U.S. Pat. No. 7,494,982.

In some embodiments, methods include subcutaneously administering one or more compositions as described herein having a telomerase inhibitor and a hyaluronidase enzyme to a subject to treat a hematological neoplasm. In some instances, treating a hematological neoplasm includes inducing apoptosis of a hematological neoplasm cell, such as inducing apoptosis of a hematological neoplasm cell in vitro. In other instances, treating a hematological neoplasm includes inducing apoptosis of a hematological neoplasm cell in a subject. In some embodiments, the hematological neoplasm cell is a malignant hematopoietic stem cell (HSC). In other embodiments, the hematological neoplasm cell is a malignant hematopoietic progenitor cell (HPC).

In some embodiments, methods include subcutaneously administering one or more compositions as described herein having a telomerase inhibitor and a hyaluronidase enzyme to a subject to treat a myeloproliferative neoplasm. In some instances, treating a myeloproliferative neoplasm includes inducing apoptosis of a myeloproliferative neoplasm cell, such as inducing apoptosis of a myeloproliferative neoplasm cell in vitro. In other instances, treating a myeloproliferative neoplasm includes inducing apoptosis of a myeloproliferative neoplasm cell in a subject. In some embodiments, the myeloproliferative neoplasm cell is a malignant hematopoietic stem cell (HSC). In other embodiments, the myeloproliferative neoplasm cell is a malignant hematopoietic progenitor cell (HPC). Myeloproliferative neoplasms treated according to the subject methods may include, for example myelofibrosis (MF), such as primary myelofibrosis, or myelofibrosis following previous ET or PV (post-ETMF or post-PVMF). In other embodiments, the myeloproliferative neoplasm includes Essential Thrombocythemia (ET), Polycythemia vera (PV), Chronic Myelogenous Leukemia (CIVIL), chronic neutrophilic leukemia, chronic eosinophilic leukemia and acute myelogenous leukemia (AML).

In other embodiments, the hematologic neoplasm is myelodysplastic syndromes (MDS). In still other embodiments, the hematologic neoplasm is myelodysplastic syndromes (MDS) with isolated non-del (5q). Myelodysplastic syndromes (MDS) include diseases such as, refractory anemia, refractory anemia with excess blasts, refractory cytopenia with multilineage dysplasia, refractory cytopenia with unilineage dysplasia, and chronic myelomonocytic leukemia (CMML). In still other embodiments, the hematological neoplasm is a lymphoid neoplasm.

Methods according to certain embodiments also include diagnosing a neoplasm. In some embodiments, methods include diagnosing a subject as having a solid tumor. In some embodiments, methods include diagnosing a subject as having a hematological neoplasm. In some embodiments, methods include diagnosing a subject as having a myeloproliferative neoplasm. In one example, methods include diagnosing the subject has having myelofibrosis, such as primary myelofibrosis. In some embodiments, the subject has not previously been administered a telomerase inhibitor (e.g., is telomerase inhibitor naïve). In some embodiments, the subject is a subject with lower risk transfusion dependent MDS who is relapsed or refractory to an erythropoietin stimulating agent (ESA). In some embodiments, the subject has not received prior treatment with a hypomethylating agent (HMA). In some embodiments, the subject has not received prior treatment with lenalidomide. In some embodiments, the subject is a subject who is non-del(5q). In some embodiments, the subject is a subject who is relapsed or refractory to a Janus kinase (JAK) inhibitor. In some embodiments, the subject methods include treating a subject having a myeloproliferative neoplasm, such as described in U.S. Pat. No. 9,375,485 and International Patent Publication Nos. WO 2019/023667 and WO 2020/028261, the disclosures of which are incorporated herein by reference.

In some embodiments the lymphoid neoplasm (e.g., lymphoma) is a B-cell neoplasm. Examples of B-cell neoplasms include, but are not limited to, precursor B-cell neoplasms (e.g., precursor B-lymphoblastic leukemia/lymphoma) and peripheral B-cell neoplasms (e.g., B-cell chronic lymphocytic leukemia, prolymphocytic leukemia, small lymphocytic lymphoma (small lymphocytic (SL) NHL), lymphoplasmacytoid lymphoma/immunocytoma, mantel cell lymphoma, follicle center lymphoma, follicular lymphoma (e.g., cytologic grades: I (small cell), II (mixed small and large cell), III (large cell) and/or subtype: diffuse and predominantly small cell type), non-Hodgkin's lymphoma (NHL), low grade/follicular non-Hodgkin's lymphoma (NHL), intermediate grade/follicular NHL, marginal zone B-cell lymphoma (e.g., extranodal (e.g., MALT-type+/−monocytoid B cells) and/or Nodal (e.g., +/− monocytoid B cells)), splenic marginal zone lymphoma (e.g., +/− villous lymphocytes), Hairy cell leukemia, plasmacytoma/plasma cell myeloma (e.g., myeloma and multiple myeloma), diffuse large B-cell lymphoma (e.g., primary mediastinal (thymic) B-cell lymphoma), intermediate grade diffuse NHL, Burkitt's lymphoma, High-grade B-cell lymphoma, Burkitt-like, high grade immunoblastic NHL, high grade lymphoblastic NHL, high grade small non-cleaved cell NHL, bulky disease NHL, AIDS-related lymphoma, and Waldenstrom's macroglobulinemia).

In some embodiments the lymphoid neoplasm (e.g., lymphoma) is a T-cell and/or putative NK-cell neoplasm. Examples of T-cell and/or putative NK-cell neoplasms include, but are not limited to, precursor T-cell neoplasm (precursor T-lymphoblastic lymphoma/leukemia) and peripheral T-cell and NK-cell neoplasms (e.g., T-cell chronic lymphocytic leukemia/prolymphocytic leukemia, and large granular lymphocyte leukemia (LGL) (e.g., T-cell type and/or NK-cell type), cutaneous T-cell lymphoma (e.g., mycosis fungoides/Sezary syndrome), primary T-cell lymphomas unspecified (e.g., cytological categories (e.g., medium-sized cell, mixed medium and large cell), large cell, lymphoepitheloid cell, subtype hepatosplenic γδ T-cell lymphoma, and subcutaneous panniculitic T-cell lymphoma), angioimmunoblastic T-cell lymphoma (AILD), angiocentric lymphoma, intestinal T-cell lymphoma (e.g., +/− enteropathy associated), adult T-cell lymphoma/leukemia (ATL), anaplastic large cell lymphoma (ALCL) (e.g., CD30+, T- and null-cell types), anaplastic large-cell lymphoma, and Hodgkin's lymphoma).

In some embodiments the lymphoid neoplasm (e.g., lymphoma) is Hodgkin's disease. For example, the Hodgkin's disease can be lymphocyte predominance, nodular sclerosis, mixed cellularity, lymphocyte depletion, and/or lymphocyte-rich.

In some embodiments, the cancer is leukemia. In some embodiments, the leukemia is chronic leukemia. Examples of chronic leukemia include, but are not limited to, chronic myelocytic I (granulocytic) leukemia, chronic myelogenous, and chronic lymphocytic leukemia (CLL). In some embodiments, the leukemia is acute leukemia. Examples of acute leukemia include, but are not limited to, acute lymphoblastic leukemia (ALL), acute myeloid leukemia, acute lymphocytic leukemia, and acute myelocytic leukemia (e.g., myeloblastic, promyelocytic, myelomonocytic, monocytic, and erythroleukemia).

In some embodiments, the cancer is liquid tumor or plasmacytoma. Plasmacytoma includes, but is not limited to, myeloma. Myeloma includes, but is not limited to, an extramedullary plasmacytoma, a solitary myeloma, and multiple myeloma. In some embodiments, the plasmacytoma is multiple myeloma.

In some embodiments, the cancer is multiple myeloma. Examples of multiple myeloma include, but are not limited to, IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma. In some embodiments, multiple myeloma may be resistant to a drug, such as, but not limited to, bortezomib, dexamethasone (Dex−), doxorubicin (Dox−), and melphalan (LR).

In describing methods of the present invention, the term “subject” is meant the person or organism to which the telomerase inhibitor composition is subcutaneously administered. As such, subjects of the invention may include but are not limited to mammals, e.g., humans and other primates, such as chimpanzees and other apes and monkey species; and the like, where in certain embodiments the subject are humans. The subject may be one that has been diagnosed as having a myeloproliferative neoplasm, where the subject may have been one that has been diagnosed by a health care professional as having the condition.

The dosage of telomerase inhibitor, such as imetelstat or imetelstat sodium that is subcutaneously administered to the subject may vary, ranging from about 2.0 mg/kg to 20.0 mg/kg, such as from about 3.0 mg/kg to about 15.0 mg/kg such as from about 4.0 mg/kg to about 10 mg/kg, such as from about 6 mg/kg to about 14 mg/kg, such as from about 7 mg/kg to about 13 mg/kg, such as from about 8 mg/kg to about 12 mg/kg, such as from about 7.5 mg/kg to 9.4 mg/kg and including from about 9 mg/kg to about 11 mg/kg and including from about 11 mg/kg to about 14 mg/kg. In some embodiments, the dosage of telomerase inhibitor administered to the subject is from about 7.5 mg/kg to about 9.4 mg/kg. In some embodiments, the dosage of telomerase inhibitor administered to the subject is from about 9 mg/kg to about 11 mg/kg. In some embodiments, the dosage of telomerase inhibitor administered to the subject is from about 11 mg/kg to about 14 mg/kg. For example, the dosage of telomerase inhibitor may be 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10 mg/kg, 10.1 mg/kg, 10.2 mg/kg, 10.3 mg/kg, 10.4 mg/kg, 10.5 mg/kg, 10.6 mg/kg, 10.7 mg/kg, 10.8 mg/kg, 10.9 mg/kg, 11 mg/kg, 11.1 mg/kg, 11.2 mg/kg, 11.3 mg/kg, 11.4 mg/kg, 11.5 mg/kg, 11.6 mg/kg, 11.7 mg/kg, 11.8 mg/kg, 11.9 mg/kg, 12 mg/kg, 12.1 mg/kg, 12.2 mg/kg, 12.3 mg/kg, 12.4 mg/kg, 12.5 mg/kg, 12.6 mg/kg, 12.7 mg/kg, 12.8 mg/kg, 12.9 mg/kg, 13 mg/kg, 13.5 mg/kg, 14.0 mg/kg, 14.5 mg/kg, 15.0 mg/kg, 15.5 mg/kg, 16.0 mg/kg, 16.5 mg/kg, 17.0 mg/kg, 17.0 mg/kg, 17.5 mg/kg, 18.0 mg/kg, 18.5 mg/kg, 19.0 mg/kg, 19.5 mg/kg, or 20.0 mg/kg. In certain embodiments, the dosage of telomerase inhibitor administered to the subject is about 9.4 mg/kg.

The dosage of the telomerase inhibitor, such as imetelstat or imetelstat sodium, may be administered to the subject in a cycle of once every other day, once every week, once every two weeks (14 days), once every three weeks (21 days) or once every four weeks (28 days), once every 6 weeks, once every 8 weeks, once every 10 weeks, once every 12 weeks. In certain embodiments of the method, imetelstat is administered for 1, 2, 3, 4, 5, 6, 7, 8 or more than 8 dosage cycles, each cycle comprising: subcutaneous administration of about 2-11 mg/kg imetelstat once every three weeks, subcutaneous administration of about 2-11 mg/kg imetelstat once every four weeks, subcutaneous administration of about 2-11 mg/kg imetelstat once every two weeks, or subcutaneous administration of about 7.5-9.4 mg/kg imetelstat once every three weeks. In certain instance, each dosage cycle comprises subcutaneous administration of about 7.5-9.4 mg/kg imetelstat once every four weeks. In some cases, each dosage cycle comprises subcutaneous administration of about 9.4 mg/kg imetelstat about once every three weeks. In some cases, each dosage cycle comprises subcutaneous administration of about 7.5 mg/kg imetelstat about once every four weeks. In certain embodiments of the method, imetelstat is administered for 1, 2, 3, 4, 5, 6, 7, 8 or more than 8 dosage cycles, each cycle comprising: subcutaneous administration of about 5-14 mg/kg imetelstat once every three weeks, subcutaneous administration of about 5-14 mg/kg imetelstat once every four weeks, subcutaneous administration of about 5-14 mg/kg imetelstat once every two weeks, or subcutaneous administration of about 7.5-14 mg/kg imetelstat once every three weeks. In certain instances, each dosage cycle comprises subcutaneous administration of about 7.5-14 mg/kg imetelstat once every four weeks. In some cases, each dosage cycle comprises subcutaneous administration of about 14 mg/kg imetelstat about once every three weeks. In some cases, each dosage cycle comprises subcutaneous administration of about 12 mg/kg imetelstat about once every four weeks.

In some embodiments, the amount of telomerase inhibitor, such as imetelstat or imetelstat sodium, administered to the individual is from about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg, about 500 mg to about 600 mg, about 600 mg to about 700 mg, about 700 mg to about 800 mg, about 800 mg to about 900 mg, about 900 mg to about 1000 mg, about 1100 mg to about 1200 mg, about 1200 mg to about 1300 mg, about 1300 mg to about 1400 mg, about 1400 mg to about 1500 mg, about 1500 mg to about 1600 mg, about 1600 mg to about 1700 mg, about 1700 mg to about 1800 mg, about 1800 mg to about 1900 mg, about 1900 mg to about 2000 mg, about 2000 mg to about 2100 mg, about 2100 mg to about 2200 mg, about 2200 mg to about 2300 mg, about 2300 mg to about 2400 mg, about 2400 mg to about 2500 mg.

In some embodiments, the amount of a telomerase inhibitor in the effective amount administered to the individual (e.g., a unit dosage form) is in the range of from about 5 mg to about 1000 mg, 5 mg to about 500 mg such as about 30 mg to about 300 mg or about 50 mg to about 200 mg. In some embodiments, the amount of a telomerase inhibitor is in a unit dosage form having an amount in the range of from about 500 mg to about 3000 mg, 750 mg to about 2500 mg, such as about 1000 mg to about 2000 mg or about 50 mg to about 200 mg. The unit dosage form may be liquid or lyophilized. In certain embodiments of the method, imetelstat is administered for 1, 2, 3, 4, 5, 6, 7, 8 or more than 8 dosage cycles, each cycle comprising: subcutaneous administration of about 200-3000 mg imetelstat once every three weeks, subcutaneous administration of about 200-3000 mg imetelstat once every four weeks, subcutaneous administration of about 750-2500 mg imetelstat once every three weeks, or subcutaneous administration of about 750-2500 mg imetelstat once every four weeks.

In some embodiments, the concentration of the telomerase inhibitor administered to the individual is dilute (about 0.1 mg/ml), or concentrated (about 300 mg/ml), including for example any of about 0.1 to about 300 mg/ml, about 0.1 to about 200 mg/ml, about 0.1 to about 180 mg/ml, about 0.1 to about 160 mg/ml, about 0.1 to about 140 mg/ml, about 0.1 to about 120 mg/ml, about 0.1 to about 100 mg/ml, about 0.1 to about 80 mg/ml, about 0.1 to about 60 mg/ml, about 0.1 to about 40 mg/ml, about 0.1 to about 20 mg/ml, about 0.1 to about 10 mg/ml about 2 to about 40 mg/ml, about 4 to about 35 mg/ml, about 6 to about 30 mg/ml, about 8 to about 25 mg/ml, about 10 to about 20 mg/ml, about 12 to about 15 mg/ml, or any of about 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2 mg/ml, 2.1 mg/ml, 2.2 mg/ml, 2.3 mg/ml, 2.4 mg/ml, or 2.5 mg/ml. In some embodiments, the concentration of the telomerase inhibitor is at least about any of 0.1 mg/ml, 0.2 mg/ml, 0.3 mg/ml, 0.4 mg/ml, 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 33.3 mg/ml, 34 mg/ml, 35 mg/ml, 36 mg/ml, 37 mg/ml, 38 mg/ml, 39 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml, 200 mg/ml, 210 mg/ml, 220 mg/ml, 230 mg/ml, 240 mg/ml, 250 mg/ml, 260 mg/ml, 270 mg/ml, 280 mg/ml, 290 mg/ml, or 300 mg/ml.

In embodiments, each dosage of telomerase inhibitor composition is subcutaneously administered to the subject once every 7 days or more, such as once every 10 days or more, such as once every 14 days or more, such as once every 21 days or more, such as once every 28 days or more and including once every 35 days or more. In some embodiments, the telomerase inhibitor composition is subcutaneously administered to the subject once every other day. In some embodiments, the telomerase inhibitor composition is subcutaneously administered to the subject once every week. In some embodiments, the telomerase inhibitor composition is subcutaneously administered to the subject once every two weeks. In other embodiments, the telomerase inhibitor composition is subcutaneously administered to the subject once every three weeks. In yet other embodiments, the telomerase inhibitor composition is subcutaneously administered once every 4 weeks.

### Kits

Also provided are kits, where kits at least include one or more, e.g., a plurality of, the subject subcutaneous telomerase inhibitor compositions, as described above. In certain embodiments, the subject subcutaneous telomerase inhibitor compositions in the kits may be provided in a package. For example, each composition of the kits may be presented in individual pouches, bottles, or analogous containers, to preserve the compositions until use. Kits may further include other components for practicing the subject methods, such as administration devices or fluids to rinse the skin before administering one or more of the subject compositions. In certain embodiments, kits include a subcutaneous injector configured to deliver a therapeutically effective amount of the composition to the subject. In some instances, the injector includes a syringe and needle. In other embodiments, the injector is a bolus injector configured to subcutaneously deliver a predetermined amount of the composition. In certain embodiments, the telomerase inhibitor composition is preloaded into the subcutaneous injector. Kits may also include gauze pads or other devices for cleaning the injection site which may find use in practicing the subject methods. In some embodiments, the telomerase inhibitor composition is formulated as a solid or lyophilate and kits may further include one or more buffer compositions or solvents for reconstituting the subject compositions for subcutaneous injection.

In addition, kits may also include instructions for how to use the subject telomerase inhibitor compositions, where the instructions may include information about to how administer the composition, dosing schedules, and record keeping devices for executing a treatment regimen. The instructions are recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the protocol for obtaining the instructions may be recorded on a suitable substrate.

### EXAMPLES

The following example is put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. By “average” is meant the arithmetic mean. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1—Bioavailability Studies of Imetelstat Sodium in Rats Via Subcutaneous and Intravenous Routes

### Materials and Methods

4 male, Sprague-Dawley rats (approximately 250 g-260 g) were dosed intravenously (IV volume 37.5 μL; 30 mg/kg) and 4 male, Sprague-Dawley rats (approximately 250 g-260 g) were dosed subcutaneously (SC volume 113 μL; 90 mg/kg) with imetelstat sodium. Imetelstat sodium is a 5′ palmitoylated 13-mer thiophosphoramidate oligonucleotide composed of the sequence 5′-TAGGGTTAGACAA-3′. Imetelstat sodium was dissolved in water immediately before dosing.

Blood was collected from each of the rats at time points of 0.083 hours (˜5 minutes), 0.25 hours (15 minutes), 1 hour, 2 hours, 4 hours, 8 hours and at 24 hours. A volume of 1.5 mL-2.0 mL of whole blood was collected, yielding about 700 μL to 1000 μL of plasma. Blood was collected through a secondary catheter in IV dosed rats (different from the one used for IV dosing).

Blood samples were collected in EDTA tubes and centrifuged immediately after collection. Collected plasma samples were transferred to a separate vial. Plasma concentration of imetelstat sodium was analyzed by LC/MS/MS.

### Results

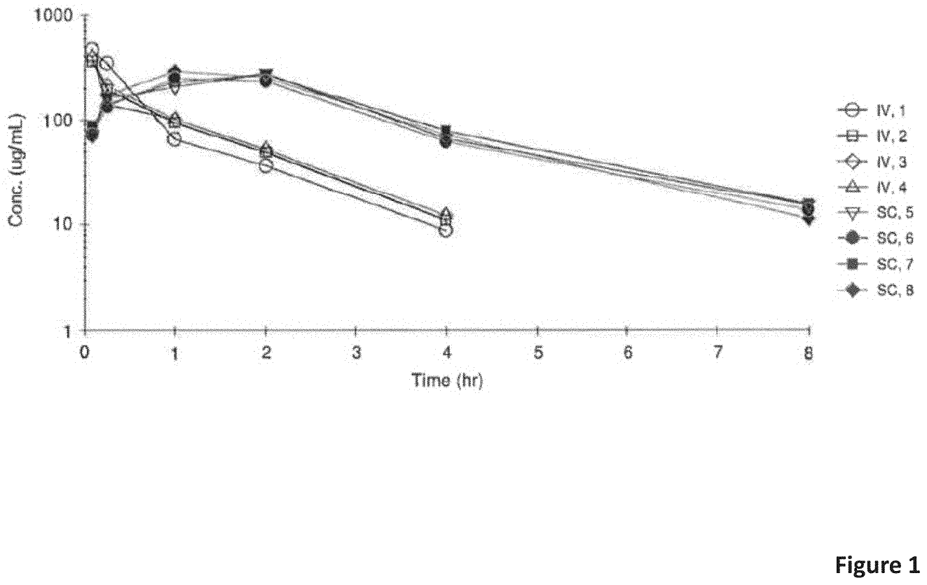

The plasma concentrations of imetelstat sodium that were analyzed over a 24 hour period are depicted in FIG. 1. The plasma concentrations of imetelstat sodium were analyzed by a noncompartmental PK analysis. The subcutaneous (s.c.) bioavailability was calculated based on the following equation: F=(AUC(s.c.)/Dose(s.c.))/(AUC(i.v.)/Dose(i.v.))×100%. The calculated subcutaneous bioavailability of imetelstat is about 95.8%. In a repeated experiment, the calculated subcutaneous bioavailability of imetelstat in rats was about 81.5%.

### Conclusions

The results shown in FIG. 1 demonstrate that imetelstat sodium was absorbed into systemic circulation after subcutaneous route of administration and showed bioavailability of approximately 80-95% compared to IV administration, supporting the use of imetelstat compositions for subcutaneous administration.

### Example 2—Compatibility and Stability of Imetelstat Sodium Incubated with Hyaluronidase

| Abbreviations | Definition |
|---|---|
| DS | Drug Substance |
| EDP | Enhanze (rHuPH20) Drug Product |
| NA | Not Applicable |
| RH | Relative Humidity |
| RRT | Relative Retention Time |
| RT | Reporting Threshold |

### Pre-Study:

Demonstration of Compatibility of Imetelstat Co-Formulated with rHuPH20

Data generated from initial feasibility testing performed as a pre-study to the following main compatibility and stability study demonstrated that results for the co-formulations of imetelstat+rHuPH20 represented by Formulations D, F and G (as defined below) prepared for this pre-study were similar to the results for the imetelstat drug substance lot without rHuPH20 used to prepare the co-formulations, providing support for compatibility of imetelstat co-formulated with rHuPH20 (see Tables below).

The following formulations were prepared

| | | Reconstituted | |
|---|---|---|---|
| | | Imetelstat | |
| Formulation | Matrix | (mg/mL) | rHuPH20 (U/mL) |
| D | 0.9% Sodium Chloride | 33 | 2,000 |
| F | 0.9% Sodium Chloride | 100 | 2,000 |
| G | EDP Buffer | 33 | 2,000 |

| | | Imetelstat + rHuPH20 Co-Formulations | | |
|---|---|---|---|---|
| Test | Imetelstat Only¹ | Formulation D | Formulation F | Formulation G |
| Appearance of | Clear solution, | Clear Solution, | Clear Solution, | Clear Solution, |
| Reconstituted | free of visible | free of visible | free of visible | free of visible |
| Solution | contaminants | contaminants | contaminants | contaminants |
| Assay by UV (μg/mg) | 1033 | 1028 | 961 | 1040 |
| Water Content | 4.2% | 4.02713% | 4.02713% | 4.02713% |
| pH (4 mL, Glass) | | 7.6 | 7.5 | 7.2 |
| pH (1 mL, Plastic) | 8.0 | 7.6 | 7.5 | 7.2 |

¹Data reflect most current stability data available for the lot of imetelstat drug substance used to prepare the imetelstat + rHuPH20 co-formulations.

| | | Imetelstat + rHuPH20 Co-Formulations | | |
|---|---|---|---|---|
| Test | Imetelstat Only¹ | Formulation D | Formulation F | Formulation G |
| Purity by RP-HPLC/UV | 93.1% | 93.0% | 93.0% | 93.0% |

Individual Impurities by RP-HPLC/UV:

| | RRT | % Area | RRT % | Area | RRT | % Area | RRT | % Area |
|---|---|---|---|---|---|---|---|---|
| Unlipidated species | 0.42 | 0.26% | 0.42 | 0.23% | 0.42 | 0.24% | 0.42 | 0.25% |
| Pre-Peak | 0.98 | 0.35% | 0.98 | 0.43% | 0.98 | 0.44% | 0.98 | 0.45% |
| Post-Peak 1 | 1.04 | 5.04% | 1.04 | 4.89% | 1.04 | 4.89% | 1.04 | 4.87% |
| Post-Peak 2 | 1.08 | 0.53% | 1.08 | 0.51% | 1.08 | 0.51% | 1.08 | 0.51% |
| Post-Peak 3 | 1.12 | <0.2% | 1.09 | 0.11% | 1.09 | 0.11% | 1.09 | 0.11% |
| Any individual | ND | ND | 0.94 | 0.16% | 0.94 | 0.16% | 0.94 | 0.16% |
| unspecified peak | | | | | | | | |
| Total Impurities by RP- | 6.9% | | 7.0% | | 7.0% | | 7.0% | |
| HPLC/UV | | | | | | | | |

¹Data reflect most current stability data available for the lot of imetelstat drug substance used to prepare the imetelstat + rHuPH20 co-formulations.

### Main Study:

### Objective

The objective of this study is to demonstrate the compatibility and stability of rHuPH20 co-formulated with imetelstat sodium under various storage conditions.

### Materials

Imetelstat Sodium
rHuPH20 Enhanze Drug Product (EDP): 1 mg/mL, 110,000 U/mL, 5° C. Storage
0.9% Sodium Chloride
L-Histidine (≥99% (TLC))
L-Methionine (reagent grade, ≥98% (HPLC))
Polysorbate 80
NaCl (ACS grade)
concentrated Hydrochloric acid (ACS reagent, 37%)
water
nylon 47 mm filter membranes 0.2 μm

### Sample Formulations

| | | Reconstituted | |
|---|---|---|---|
| | | Imetelstat | rHuPH20 |
| Formulation | Matrix | (mg/mL) | (U/mL) |
| A (Imetelstat Control) | 0.9% Sodium Chloride | 33 | 0 |
| B (rHuPH20 | 0.9% Sodium Chloride | 0 | 2,000 |
| Saline Control) | | | |
| C (Imetelstat EDP | EDP Buffer | 33 | 0 |
| buffer Control) | | | |
| D | 0.9% Sodium Chloride | 33 | 2,000 |
| E | 0.9% Sodium Chloride | 33 | 1,000 |
| F | 0.9% Sodium Chloride | 100 | 2,000 |
| G | EDP Buffer | 33 | 2,000 |

### Sample Preparation

| | | | Volume of | | |
|---|---|---|---|---|---|
| | | | rHuPH20 | | |
| | | Recon- | (mL) added | Volume | Volume of |
| | | stituted | from a 1 | of | Imetelstat |
| Formu- | | Imetelstat | mg/mL | Imetelstat | Matrix |
| lation | Matrix | (mg/mL) | stock | (mL) | (mL) |
| A | 0.9% | 33 | 0.0 | 150 | 0 |
| (Imetelstat | Sodium | | | | |
| Control) | Chloride | | | | |
| B | 0.9% | 0 | 2.7 | 0 | 147.3 |
| (rHuPH20 | Sodium | | | | |
| Saline | Chloride | | | | |
| Control) | | | | | |
| C | EDP | 33 | 0.0 | 150 | 0 |
| (Imetelstat | Buffer | | | | |
| EDP buffer | | | | | |
| Control) | | | | | |
| D | 0.9% | 33 | 2.7 | 147.3 | 0 |
| | Sodium | | | | |
| | Chloride | | | | |
| E | 0.9% | 33 | 1.4 | 148.6 | 0 |
| | Sodium | | | | |
| | Chloride | | | | |
| F | 0.9% | 100 | 2.7 | 147.3 | 0 |
| | Sodium | | | | |
| | Chloride | | | | |
| G | EDP | 33 | 2.7 | 147.3 | 0 |
| | Buffer | | | | |

Preparation of rHuPH20 Enhanze Drug Product (EDP) Buffer

Approximately 450 mL water is added into 500 mL volumetric flask. About 776 mg (±15 mg) L-histidine, about 3.80 g NaCl (±75 mg), and about 746 mg (±15 mg) L-methionine is added into the volumetric flask. The components are dissolved completely using a stir bar. Visual inspection is used to verify that the components are dissolved.

To the solution, 1 mL of the 10.0% polysorbate 80 composition is added to the volumetric flask. The composition is mixed with a stir bar. The pH is adjusted using concentrated HCl to a final pH of 6.5±0.3. QS to final volume of 500 mL. The composition is filtered through a nylon 47 mm filter membrane 0.2 μm and stored at 5° C. and is stable for at least 2 weeks.

The composition is stored in 20 mL borosilicate glass scintillation vials at 5° C. in upright orientation, 25° C. in upright orientation and 37° C. in upright orientation.

### Stability Protocol Schedule

The following Table summarizes the number of samples set down per Interval/Condition/Formulation (Initial Interval Samples are stored at −20° C. conditions)

| | | 5° C. | 25° C. | 37° C. |
|---|---|---|---|---|
| Stability | | Upright | Upright | Upright |
| Interval | | Orientation | Orientation | Orientation |
| Initial | | 6 | | |
| 4 | hours | 2 | 2 | 2 |
| 8 | hours | 2 | 2 | 2 |
| 24 | hours | 2 | 2 | — |
| 48 | hours | 2 | 2 | — |
| 1 | weeks | 2 | 2 | — |
| 2 | weeks | 2 | 2 | — |
| 3 | months | 2 | 2 | — |
| 6 | months | 2 | 2 | — |
| 9 | months | 2 | 2 | — |
| 12 | months | 2 | 2 | — |
| Reserves | | 2 | 2 | 0 |
| Total | | 28 | 22 | 4 |

### Stability Pull and Testing Protocol

The testing is performed on samples set down per the designated time intervals and conditions in order to assess the compatibility and stability of co-formulations of imetelstat+rHuPH20 (See Table below). The test methods include assessments of appearance, pH, oligo concentration (UV), purity by HPLC, assay by LC/MS, rHuPH20 enzyme activity, and telomerase activity for imetelstat by TRAP assay (Mender and Shay, Bio Protoc. 2015: 5(22)).

| | | 5° C. | 25° C. | 37° C. |
|---|---|---|---|---|
| Stability | | Upright | Upright | Upright |
| Interval | | Orientation | Orientation | Orientation |
| Initial | | ABC | | |
| 4 | hours | NA | NA | A |
| 8 | hours | A | A | AB |
| 24 | hours | A | A | NA |
| 48 | hours | A | A | NA |
| 1 | weeks | A | A | NA |
| 2 | weeks | AB | AB | NA |
| 3 | months | R | R | NA |
| 6 | months | R | R | NA |
| 9 | months | R | R | NA |
| 12 | months | R | R | NA |

One aliquot of T = 0 sample for conditions A, D, and F to be assessed by TRAP assay upon collection; a second aliquot to be retained for possible future analysis together with samples from other conditions and/or timepoints.
A = Appearance, pH, Oligo concentration (UV), RP-HPLC, Enzyme activity assay for rHuPH20
B = LC/MS
C = Telomerase activity (TRAP) assay for Imetelstat
R = Samples to be tested as needed

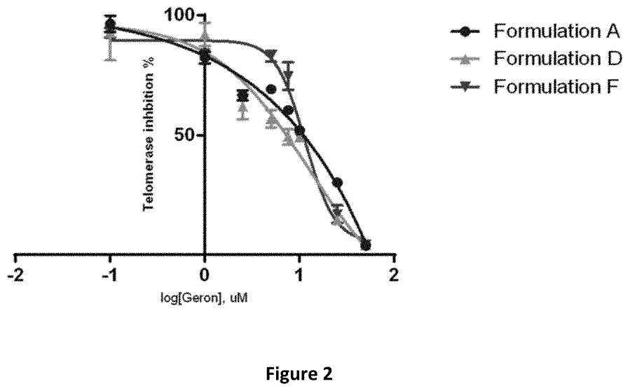

To test if rHuPH20 would have any impact on imetelstat telomerase inhibiting activity when co-mixed with imetelstat, three formulations A, D and F as indicated in the Sample Formulations Table above, i.e., imetelstat sodium alone (Formulation A) or imetelstat sodium co-mixed with rHuPH20 (Formulations D and F), were tested in a TRAP (Telomerase Repeated Amplification Protocol) assay for imetelstat activity. Briefly, cancer cell line Hela cells were treated in vitro in triplicate with seven (7) different concentrations (0.1, 1, 5, 7.5, 10, 25 and 50 μM) of each test Formulation, and one (1) no-drug control for 24 hours. The cell pellets were lysed for protein extraction and quantification and the same amount of protein from cells treated with each concentration of Formulation were tested by the TRAP assay. The Relative Telomerase Activity (RTA) for each of the quantitative polymerase chain reaction (qPCR) results was determined from a standard curve, which was generated by a TRAP assay of serial dilutions of the protein lysate from untreated Hela cells. The average RTA from replicates were calculated for each concentration of a given Formulation and compared to the average RTA value of the no-drug control to generate the percent (%) of telomerase activity inhibition, and plotted against the treatment concentration to determine the drug concentrations for each Formulation to achieve 50% inhibition (IC50 value) of telomerase activity. Results in FIG. 2 showed that the IC50 values for all 3 Formulations were very similar and close to log value of 1 (10 μM), indicating that co-mixing imetelstat sodium with rHuPH20 does not affect imetelstat's telomerase inhibition activity.

### Example 3—Assay of rHuPH20 Activity in Formulations of Imetelstat and rHuPH20

### Overview

In additional feasibility testing performed as a pre-study to the main compatibility and stability study described in Example 2 above, formulations having imetelstat co-mixed with rHuPH20 were tested for rHuPH20 activity. Results from the assay based on the reaction of rHuPH20 with biotinylated hyaluronic acid (HA) are provided and demonstrate that rHuPH20 activity can be measured in the presence of imetelstat in the co-mixed samples.

### Results

Biotinylated hyaluronic acid was used to determine the activity of rHuPH20 in the presence and absence of imetelstat. Compositions containing 2000 U/mL of rHuPH20 in saline were assayed with biotinylated hyaluronic acid to measure the level of degradation of the hyaluronic acid by rHuPH20 and compared to compositions containing 100 mg/mL imetelstat with 2000 U/mL rHuPH20. The rHuPH20 activity detected in the samples for each imetelstat+rHuPH20 composition was close to amounts based on activities seen with the rHuPH20 alone samples (Table below).

| | 2000 U/mL | | 100 mg/mL imetelstat with | |
|---|---|---|---|---|
| | rHuPH20 in saline | | 2000 U/mL rHuPH20 | |
| Target U/mL | Potency | % Recovery | Potency | % Recovery |
| in assay | (U/mL) | (2000 U/mL) | (U/mL) | (2000 U/mL) |
| 1 | 1526 | 76% | 1022 | 51% |
| 0.33 | 1860 | 93% | 2394 | 120% |
| 0.11 | 2124 | 106% | 2916 | 146% |
| 0.037 | 1944 | 97% | 2592 | 130% |
| 0.012 | 2268 | 113% | 3078 | 154% |
| 0.004 | 3402 | 170% | 3402 | 170% |

### Example 4—Single Dose Subcutaneous Bioavailability and Local Tolerability Study of Imetelstat with or without rHuPH20 in Rats

### Objective

The objectives of this study are to assess the systemic uptake of imetelstat (in terms of plasma levels) and the local (injection site) tolerability following a single subcutaneous (SC) injection alone or in combination with rHuPH20 (a recombinant human hyaluronidase enzyme product to be co-formulated with imetelstat) in rats. The subcutaneous bioavailability of imetelstat is determined by including a group that receives an IV dose of this test article. The study is designed to demonstrate that imetelstat co-formulated with rHuPH20 is tolerable and provides a pharmacokinetic profile for imetelstat indicative of clinical use of imetelstat co-formulated with rHuPH20 to deliver requisite liquid volumes containing sufficient doses of imetelstat via the subcutaneous route of administration.

### Animals

Sprague-Dawley rats, each having a standard age at the outset of the study.

### Test Articles

Imetelstat is tested with and without rHuPH20.

Study Design/Dosing

Imetelstat is given as a single dose on Day 1 either by slow-push intravenous injection (Group 1) or SC injection (Group 2). Imetelstat co-formulated with rHuPH20 is given as a single dose on Day 1 by SC injection (Groups 3a and 3b). The vehicle for imetelstat is given concurrently to Group 2 animals at a different SC site (control injection site). Likewise, the vehicle for imetelstat co-formulated with rHuPH20 is given to Groups 3a and 3b animals at a separate control injection site.

Group assignments are shown in the table below.

| | | | Solution | | Dosing Volume | |
|---|---|---|---|---|---|---|
| | | Dose | Concentration | | Imetelst | Vehicle |
| Group | | Imetelstat | Imetelstat | rHuPH20 | at | Control |
| No. | Route | (mg/kg) | (mg/mL) | (units/mL) | (mL/kg) | Injection (mL) |
| 1 | IV | 20-30 | 15 | — | 1.3-2 | N/A |
| 2 | SC | 20-30 | 100 | — | 0.2-0.3 | Same volume |
| | | | | | | as test |
| | | | | | | article* |
| 3a | SC | 20-30 | 3 | 2000 | 6.6-10 | Same volume |
| 3b | SC | 20-30 | 100 | 2000 | 0.2-0.3 | as test |
| | | | | | | article** |

SC: subcutaneous
*Vehicle control article for imetelstat, which is given at a different SC site than imetelstat dose (at the same dose volume as imetelstat alone)
**Vehicle control article for imetelstat/rHuPH20 co-formulation, which is given at a different SC site than imetelstat/rHuPH20 co-formulation dose.

### Observations and Sample Collection

Clinical Observations: Study observations include standard clinical observations, assessment of local injection sites, food consumption and body weight.

### PK Samples:

Blood samples are collected from all animals in K₂EDTA-containing tubes at various times post-dose from 5 minutes to 8 hours. The samples are processed to plasma under refrigerated conditions and the resulting plasma are stored deep-frozen.

### PK Sample Bioanalysis and Data Interpretation:

The rat plasma samples are analyzed for imetelstat concentration using a validated hybridization ELISA method. PK parameters such as AUC₀₋ₜ, AUC_{0-inf}, Cₘₐₓ, Tₘₐₓ, Kₑₗ, CL, V_{d} and t_{1/2} are calculated for imetelstat in plasma as appropriate.

### Example 5—Single Dose Subcutaneous Pharmacokinetic and Local Tolerability Study in Monkeys

### Objective

The objectives of this study are to assess the systemic uptake of imetelstat (in terms of plasma levels) and the local (injection site) tolerability following a single subcutaneous injection alone or in combination with rHuPH20 (a recombinant human hyaluronidase enzyme product to be co-formulated with imetelstat) in cynomolgus monkeys. The study is designed to demonstrate that imetelstat co-formulated with rHuPH20 is tolerable and provides a pharmacokinetic profile for imetelstat indicative of clinical use of imetelstat co-formulated with rHuPH20 to deliver requisite liquid volumes containing sufficient doses of imetelstat via the subcutaneous route of administration.

### Animals

Cynomolgus monkeys of standard age and weight at the outset of the study.

### Test Articles

Imetelstat is tested with and without rHuPH20.

### Study Design/Dosing

Imetelstat is given as a single dose on Day 1 by SC injection (Group 1), and imetelstat co-formulated with rHuPH20 is given as a single dose on Day 1 by subcutaneous injection (Group 2). The vehicle for imetelstat is given concurrently to Group 1 animals at a different subcutaneous site (control injection site). Likewise, the vehicle for the co-formulation of imetelstat and rHuPH20 is given to Group 2 animals at a separate control site.

Group assignments are shown in the table below.

| | | | | | Dosing Volume | |
|---|---|---|---|---|---|---|
| | | | Solution | | | Vehicle |
| | | Dose | Concentration | | | Control |
| Group | | Imetelstat | Imetelstat | rHuPH20 | Imetelstat | Injection |
| No. | Route | (mg/kg) | (mg/mL) | (units/mL) | (mL/kg) | (mL) |
| 1 | SC | 15 | 100 | — | 0.15 | Same |
| | | | | | | volume as |
| | | | | | | test article |
| | | | | | | * |
| 2 | SC | 15 | 100 | 2000 | 0.15 | Same |
| | | | | | | volume as |
| | | | | | | test article |
| | | | | | | ** |

SC: subcutaneous
* Vehicle control article for imetelstat, which is given at a different SC site than imetelstat dose.
** Vehicle control article for imetelstat/rHuPH20 co-formulation, which is given at a different SC site than imetelstat/rHuPH20 co-formulation dose.

### Observations and Sample Collection

### Clinical Observations:

Study observations to include standard clinical observations, assessment of local injection sites (including histopathology examination), food consumption and body weight.

### PK Samples:

Blood samples are collected from all animals in K₂EDTA-containing tubes at the various times post-dose from 5 minutes to 24 hours. The samples are processed to plasma under refrigerated conditions and the resulting plasma are stored deep-frozen.

### PK Sample Bioanalysis and Data Interpretation:

The monkey plasma samples are analyzed for imetelstat concentration using a validated hybridization ELISA method. PK parameters such as AUC₀₋ₜ, AUC_{0-inf}, Cₘₐₓ, Tₘₐₓ, Kₑₗ, CL, V_{d} and t_{1/2} are calculated for imetelstat in plasma as appropriate.

### Example 6—Safety and Pharmacokinetics Study in Healthy Volunteers or Patients

### Objective

The objectives of this study are to assess the safety, tolerability and pharmacokinetics of imetelstat after subcutaneous (SC) and intravenous (IV) administration. The study is designed to demonstrate co-formulations of imetelstat with rHuPH20 provides subcutaneous delivery of requisite liquid volumes containing sufficient doses of imetelstat to demonstrate acceptable safety, tolerability and pharmacokinetic profile for imetelstat via the subcutaneous route of administration.

### Summary of Study Design

The study is conducted in two parts. Part 1 is a dose-escalation phase with subcutaneous administration of imetelstat. In Part 1, up to about 4 cohorts are planned. Subjects participate in only 1 cohort. In each cohort, subjects receive a single subcutaneous dose of imetelstat on Day 1. Part 2 is an open label, randomized, 2-treatment, crossover or parallel study design. The dose for Part 2 is selected after review of the safety and pharmacokinetics data from Part 1.

If Part 2 is a crossover design, on Day 1 of Periods 1 and 2, alternating subcutaneous or intravenous doses of imetelstat is administered according to a randomization schedule, followed by pharmacokinetic sampling for 48 hours. There is a planned washout period between the imetelstat doses in Part 2 in the crossover design. If Part 2 is a parallel design, patients are randomized to receive on Day 1 either a subcutaneous or intravenous dose of imetelstat, followed by pharmacokinetic sampling for 48 hours.

### Dosage, Dosage Form, Route and Dosage Regimen

Subcutaneous imetelstat is formulated as a co-mixture with hyaluronidase (rHuPH20) in an appropriate vehicle. IV imetelstat does not contain hyaluronidase and is formulated in 0.9% sodium chloride.

### Part 1:

Single subcutaneous doses to be administered in Part 1. Cohort doses are selected based on review of safety and pharmacokinetics data from preclinical pharmacokinetics and local tolerability studies in rats and monkeys.

### Part 2:

Subjects are administered imetelstat subcutaneous or IV in one of the following sequences:
Sequence A (Crossover Design): A single subcutaneous dose of imetelstat on Day 1 followed by a single IV dose of imetelstat after an appropriate washout period; or
Sequence B (Crossover Design): A single IV dose of imetelstat on Day 1 followed by a single SC dose of imetelstat after an appropriate washout period; or
Sequence C (Parallel Design): A single subcutaneous dose of imetelstat on Day 1 administered to one cohort of subjects and a single IV dose of imetelstat on Day 1 administered to another cohort of subjects.

The subcutaneous dose is given as a single injection at one site, and the IV dose is given as a 2-hour infusion.

### Assessments

### Pharmacokinetics:

Blood samples for plasma concentrations of imetelstat are collected pre-dose, and at various times from 0.5 to 48 hours after subcutaneous dose or start of infusion.

The following PK parameters are calculated for imetelstat in plasma, as appropriate: AUC₀₋ₜ, AUC_{0-inf}, AUC %ₑₓₜᵣₐₚ, Cₘₐₓ, Tₘₐₓ, Kₑₗ, C_{L}, C_{L/F}, and t_{1/2}.

### Safety:

Assessment of Safety and tolerability is monitored through standard procedures which may include electrocardiograms (ECGs), physical examinations, vital sign measurements, clinical laboratory tests, and AEs. Summary statistics may be computed as deemed clinically appropriate.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a feature in the claim only when the exact phrase “means for” or the exact phrase “step for” is recited at the beginning of such feature in the claim; if such exact phrase is not used in a feature in the claim, then 35 U.S.C. § 112(f) or 35 U.S.C. § 112(6) is not invoked.

Other embodiments of the invention may include:

1. A composition formulated for subcutaneous administration, the composition comprising:

a telomerase inhibitor comprising an oligonucleotide and a lipid moiety linked to the 5′ and/or 3′ end of the oligonucleotide; and

a hyaluronidase enzyme.

2. The composition according to paragraph 1, wherein the hyaluronidase enzyme is a recombinant human hyaluronidase.
3. The composition according to paragraph 2, wherein the hyaluronidase enzyme is rHuPH20.
4. The composition according to paragraph 1, wherein the composition comprises a variant or fragment of a PH20 hyaluronidase enzyme.
5. The composition according to paragraph 4, wherein the variant or fragment of PH20 comprises one or more amino acid residue substitutions selected from the group consisting of T341A, T341C, T341G, S343E, M345T, K349E, L353A, L354I, N356E and I361T in wild-type PH20 having the amino acid sequence of SEQ ID NO: 1.
6. The composition according to paragraph 5, wherein the variant or fragment of PH20 comprises one or more amino acid residue substitutions selected from the group consisting of T341A, T341C, L354I and N356E.
7. The composition according to paragraph 4, wherein the variant or fragment of PH20 comprises one or more amino acid residue substitutions in the region corresponding to an alpha-helix region or a linker region in wild-type PH20 having the amino acid sequence of SEQ ID NO: 1.
8. The composition according to paragraph 7, wherein the alpha-helix region is an alpha-helix 8 region comprising the amino acid residues S347 to C381 and the linker region is a linker region comprising the amino acid residues A333 to R346 between alpha-helix 7 and alpha-helix 8.
9. The composition according to paragraph 7, wherein the alpha-helix region and the linker region comprises amino acid residues T341 to N363, T341 to I361, L342 to I361, S343 to I361, I344 to I361, M345 to I361, or M345 to N363.
10. The composition according to paragraph 7, wherein the alpha-helix 8 region and the linker region between alpha-helix 7 and alpha-helix 8 are substituted with one or more amino acid residues of the corresponding region of Hyal1.
11. The composition according to paragraph 4, wherein the variant or fragment of PH20 comprises one or more amino acid residue substitutions at one or more positions selected from the group consisting of T341, L342, S343, I344, M345, S347, M348, K349, L352, L353, D355, E359, I361 and N363.
12. The composition according to paragraph 11, wherein the variant or fragment of PH20 comprises amino acid residue substitutions of:

one or more of L354I and N356E; and

one or more amino acid residue substitutions selected from the group consisting of T341A, T341C, T341D, T341G, T341S, L342W, S343E, I344N, M345T, S347T, M348K, K349E, L352Q, L353A, D355K, E359D, I361T and N363G.

13. The composition according to paragraph 12, wherein the variant or fragment of PH20 comprises

(i) the substitutions M345T, S347T, M348K, K349E, L352Q, L353A, L354I, D355K, N356E, E359D and I361T;

(ii) the substitutions T341A, T341C, T341D, T341G, T341S, L342W, S343E, I344N and N363G; or

(iii) any one amino acid residue substitution selected from the following amino acid residue substitution groups:
(a) T341S, L342W, S343E, I344N, M345T, S347T, M348K, K349E, L352Q, L353A, L354I, D355K, N356E, E359D and I361T;
(b) L342W, S343E, I344N, M345T, S347T, M348K, K349E, L352Q, L353A, L354I, D355K, N356E, E359D and I361T;
(c) M345T, S347T, M348K, K349E, L352Q, L353A, L354I, D355K, N356E, E359D, I361T and N363G;
(d) T341G, L342W, S343E, I344N, M345T, S347T, M348K, K349E, L352Q, L353A, L354I, D355K, N356E, E359D and I361T;
(e) T341A, L342W, S343E, I344N, M345T, S347T, M348K, K349E, L352Q, L353A, L354I, D355K, N356E, E359D and I361T;
(f) T341C, L342W, S343E, I344N, M345T, S347T, M348K, K349E, L352Q, L353A, L354I, D355K, N356E, E359D and I361T;
(g) T341D, L342W, S343E, I344N, M345T, S347T, M348K, K349E, L352Q, L353A, L354I, D355K, N356E, E359D and I361T;
(h) I344N, M345T, S347T, M348K, K349E, L352Q, L353A, L354I, D355K, N356E, E359D and I361T; and
(i) S343E, I344N, M345T, S347T, M348K, K349E, L352Q, L353A, L354I, D355K, N356E, E359D and I361T.
   14. The composition according to any one of paragraphs 4-13, wherein one or more of the N-terminal or C-terminal amino acid residues of the variant or fragment of PH20 are deleted.
   15. The composition according to paragraph 14, wherein the cleavage

(i) is positioned before an amino acid residue selected from the group consisting of M1 to P42 at the N-terminus such that one or more residues at the N-terminus are deleted;

(ii) is positioned before an amino acid residue L36, N37, F38, R39, A40, P41, or P42 at the N-terminus such that one or more residues at the N-terminus are deleted;

(iii) is positioned after an amino acid residue selected from the group consisting of V455 to L509 at the C-terminus such that one or more amino acid residues at the C-terminus are deleted; or

(iv) is positioned after an amino acid residue selected from V455, C458, D461, C464, I465, D466, A467, F468, K470, P471, P472, M473, E474, T475, E476, E477, P478, Q479, I480, F481, Y482, N483, A484, P486, T488, or S490 at the C-terminus such that one or more amino acid residues at the C-terminus are deleted.

16. The composition according to any one of paragraphs 4 to 15, wherein the N-terminus comprises a human growth hormone-derived signal peptide having an amino acid sequence MATGSRTSLLLAFGLLCLPWLQEGSA of SEQ ID NO: 3, a human serum albumin-derived signal peptide having an amino acid sequence MKWVTFISLLFLFSSAYS of SEQ ID NO: 4, or a human Hyal1-derived signal peptide having an amino acid sequence

| of SEQ ID NO: 5 | |
|---|---|
| | MAAHLLPICALFLTLLDMAQG. |

22. The composition according to any one of paragraphs 4-21, wherein the variant or fragment of PH20
(i) is a peptide having at least 90% sequence identity to a sequence of amino acids set forth as SEQ ID NO:1 or amino acid residues 36-482, 36-477, 366-478, 36-479, 36-480, 36-481, and 36-483 of SEQ ID NO:1;
(ii) is a peptide having at least 95% sequence identity to a sequence of amino acids set forth as SEQ ID NO:1 or amino acid residues 36-482, 36-477, 366-478, 36-479, 36-480, 36-481, and 36-483 of SEQ ID NO:1;
(iii) consists of an amino acid sequence selected from SEQ ID NOs: 60 to 115; (iv) has an amino acid sequence of SEQ ID NO: 99.
   23. The composition according to any one of paragraphs 1-22, wherein the hyaluronidase is present in the composition in an amount of from 100 U to 50,000 U.
   24. The composition according to any one of paragraphs 1-23, wherein the hyaluronidase is present in the composition in an amount of from 100 U/mL to 50,000 U/mL.
   25. The composition according to any one of paragraphs 1-24, wherein the composition further comprises one or more pharmaceutically acceptable excipients.
   26. The composition according to any one of paragraphs 1-25, wherein the composition further comprises one or more saccharides.
   27. The composition according to paragraph 26, wherein the one or more saccharides comprises a monosaccharide.
   28. The composition according to any one of paragraphs 26-27, wherein the one or more saccharides comprises a polysaccharide.
   29. The composition according to paragraph 28, wherein the one or more polysaccharides is selected from the group consisting of trehalose and sucrose.
   30. The composition according to any one of paragraphs 26-29, wherein the one or more saccharides is present in the composition in an amount from 10 mM to 500 mM.
   31. The composition according to any one of paragraphs 1-30, wherein the composition further comprises one or more amino acids.
   32. The composition according to paragraph 31, wherein the amino acids are selected from methionine and histidine.
   33. The composition according to any one of paragraphs 31-32, wherein the one or more amino acids is present in the composition in an amount from 1 mM to 100 mM, optionally in an amount from 1 mM to 50 mM.
   34. The composition according to any one of paragraphs 1-33, wherein the composition further comprises a buffer.
   35. The composition according to paragraph 34, wherein the buffer is present in the composition in an amount sufficient to maintain the composition at a pH from 3.0 to 9.0, optionally in an amount sufficient to maintain the composition at a pH from 5.5 to 7.5.
   36. The composition according to any one of paragraphs 34-35, wherein the buffer is present in the composition in an amount of from 1 to 100 mM, optionally in an amount of from 1 mM to 50 mM.
   37. The composition according to any one of paragraphs 1-36, wherein the oligonucleotide of the telomerase inhibitor comprises at least one N3′ 4 P5′ thiophosphoramidate internucleoside linkage.
   38. The composition according to any one of paragraphs 1-37, wherein the lipid moiety of the telomerase inhibitor is linked to the 5′ and/or 3′ end of the oligonucleotide via a linker, optionally wherein the linker is a glycerol or aminoglycerol linker.
   39. The composition according to any one of paragraphs 1-38, wherein the lipid moiety of the telomerase inhibitor is a palmitoyl (C16) moiety.
   40. The composition according to any one of paragraphs 1-39, wherein the telomerase inhibitor is imetelstat or a pharmaceutically acceptable salt thereof, optionally wherein the telomerase inhibitor is imetelstat sodium.
   41. The composition according to any one of paragraphs 1-40, wherein the telomerase inhibitor is present in the composition at a dosage

(i) of from about 2.0 mg/kg to 20.0 mg/kg;

(ii) of from about 3 mg/kg to about 15 mg/kg;

(iii) of from about 9 mg/kg to about 11 mg/kg; or

(iv) of from about 11 mg/kg to about 14 mg/kg.

42. The composition according to paragraphs 1-41, wherein the composition is lyophilized.
43. A method of treating a subject having a neoplasm, the method comprising subcutaneously administering to the subject a composition comprising:

a telomerase inhibitor comprising an oligonucleotide and a lipid moiety linked to the 5′ and/or 3′ end of the oligonucleotide; and

a hyaluronidase enzyme.

44. The method according to paragraph 43, wherein the neoplasm is a hematologic neoplasm selected from myelofibrosis (MF), myelodysplastic syndromes (MDS), Essential Thrombocythemia (ET), Polycythemia vera (PV), Chronic Myelogenous Leukemia (CIVIL), chronic neutrophilic leukemia, chronic eosinophilic leukemia, and acute myeloid leukemia (AML).
45. The method according to any one of paragraphs 43-44, further comprising diagnosing a subject as having a hematologic neoplasm.
46. The method according to any one of paragraphs 43-45, wherein the composition is subcutaneously administered to the subject

(i) once every other day;

(ii) once every 7 days;

(iii) once every 21 days; or

(iv) once every 28 days.

47. The method according to any one of paragraphs 43-46, wherein the method is repeated 1 or more times, optionally 5 or more times.
48. The method according to any one of paragraphs 43-47, wherein the telomerase inhibitor is administered to the subject at a dosage of from

(i) about 2.0 mg/kg to 20.0 mg/kg;

(ii) about 3 mg/kg to about 15 mg/kg;

(iii) about 9 mg/kg to about 11 mg/kg; or

(iv) about 11 mg/kg to about 14 mg/kg.

49. The method according to any one of paragraphs 43-48, wherein the hyaluronidase enzyme is a recombinant human hyaluronidase, optionally rHuPH20.
50. The method according to any one of paragraphs 43-48, wherein the composition comprises a variant or fragment of a PH20 hyaluronidase enzyme.
51. The method according to paragraph 50, wherein the variant or fragment of PH20 comprises one or more amino acid residue substitutions selected from the group consisting of T341A, T341C, T341G, S343E, M345T, K349E, L353A, L354I, N356E and I361T in wild-type PH20 having the amino acid sequence of SEQ ID NO: 1, optionally wherein the variant or fragment of PH20 comprises one or more amino acid residue substitutions selected from the group consisting of T341A, T341C, L354I and N356E.
52. The method according to paragraph 50, wherein the variant or fragment of PH20 comprises one or more amino acid residue substitutions in the region corresponding to an alpha-helix region or a linker region in wild-type PH20 having the amino acid sequence of SEQ ID NO: 1.
53. The method according to paragraph 52, wherein

(i) the alpha-helix region is an alpha-helix 8 region comprising the amino acid residues S347 to C381 and the linker region is a linker region comprising the amino acid residues A333 to R346 between alpha-helix 7 and alpha-helix 8;

(ii) the alpha-helix region and the linker region comprises amino acid residues T341 to N363, T341 to I361, L342 to I361, S343 to I361, I344 to I361, M345 to I361, or M345 to N363; or

(iii) the alpha-helix 8 region and the linker region between alpha-helix 7 and alpha-helix 8 are substituted with one or more amino acid residues of the corresponding region of Hyal1.

54. The method according to paragraph 50, wherein the variant or fragment of PH20 comprises one or more amino acid residue substitutions at one or more positions selected from the group consisting of T341, L342, S343, I344, M345, S347, M348, K349, L352, L353, D355, E359, I361 and N363.
55. The method according to paragraph 54, wherein the variant or fragment of PH20 comprises amino acid residue substitutions of:

one or more of L354I and N356E; and

one or more amino acid residue substitutions selected from the group consisting of T341A, T341C, T341D, T341G, T341S, L342W, S343E, I344N, M345T, S347T, M348K, K349E, L352Q, L353A, D355K, E359D, I361T and N363G,

optionally wherein the variant or fragment of PH20 comprises the substitutions M345T, S347T, M348K, K349E, L352Q, L353A, L354I, D355K, N356E, E359D and I361T.

56. The method according to paragraph 55, wherein the variant or fragment of PH20 comprises

(i) the substitutions T341A, T341C, T341D, T341G, T341S, L342W, S343E, I344N and N363G; or

(ii) any one amino acid residue substitution selected from the following amino acid residue substitution groups:

(a) T341S, L342W, S343E, I344N, M345T, S347T, M348K, K349E, L352Q, L353A, L354I, D355K, N356E, E359D and I361T;

(b) L342W, S343E, I344N, M345T, S347T, M348K, K349E, L352Q, L353A, L354I, D355K, N356E, E359D and I361T;

(c) M345T, S347T, M348K, K349E, L352Q, L353A, L354I, D355K, N356E, E359D, I361T and N363G;

(d) T341G, L342W, S343E, I344N, M345T, S347T, M348K, K349E, L352Q, L353A, L354I, D355K, N356E, E359D and I361T;

(e) T341A, L342W, S343E, I344N, M345T, S347T, M348K, K349E, L352Q, L353A, L354I, D355K, N356E, E359D and I361T;

(f) T341C, L342W, S343E, I344N, M345T, S347T, M348K, K349E, L352Q, L353A, L354I, D355K, N356E, E359D and I361T;

(g) T341D, L342W, S343E, I344N, M345T, S347T, M348K, K349E, L352Q, L353A, L354I, D355K, N356E, E359D and I361T;

(h) I344N, M345T, S347T, M348K, K349E, L352Q, L353A, L354I, D355K, N356E, E359D and I361T; and

(i) S343E, I344N, M345T, S347T, M348K, K349E, L352Q, L353A, L354I, D355K, N356E, E359D and I361T.

57. The method according to any one of paragraphs 50-56, wherein one or more of the N-terminal or C-terminal amino acid residues of the variant or fragment of PH20 are deleted.
58. The method according to paragraph 57, wherein cleavage is positioned

(i) before an amino acid residue selected from the group consisting of M1 to P42 at the N-terminus such that one or more residues at the N-terminus are deleted;

(ii) before an amino acid residue L36, N37, F38, R39, A40, P41, or P42 at the N-terminus such that one or more residues at the N-terminus are deleted;

(iii) positioned after an amino acid residue selected from the group consisting of V455 to L509 at the C-terminus such that one or more amino acid residues at the C-terminus are deleted; or

(iv) after an amino acid residue selected from V455, C458, D461, C464, I465, D466, A467, F468, K470, P471, P472, M473, E474, T475, E476, E477, P478, Q479, I480, F481, Y482, N483, A484, P486, T488, or S490 at the C-terminus such that one or more amino acid residues at the C-terminus are deleted.

59. The method according to any one of paragraphs 50-58, wherein the N-terminus comprises a human growth hormone-derived signal peptide having an amino acid sequence MATGSRTSLLLAFGLLCLPWLQEGSA of SEQ ID NO: 3, a human serum albumin-derived signal peptide having an amino acid sequence MKWVTFISLLFLFSSAYS of SEQ ID NO: 4, or a human Hyal1-derived signal peptide having an amino acid sequence MAAHLLPICALFLTLLDMAQG of SEQ ID NO: 5.
60. The method according to any one of paragraphs 50-59, wherein the variant or fragment of PH20

(i) is a peptide having at least 90% sequence identity to a sequence of amino acids set forth as SEQ ID NO:1 or amino acid residues 36-482, 36-477, 366-478, 36-479, 36-480, 36-481, and 36-483 of SEQ ID NO:1;

(ii) is a peptide having at least 95% sequence identity to a sequence of amino acids set forth as SEQ ID NO:1 or amino acid residues 36-482, 36-477, 366-478, 36-479, 36-480, 36-481, and 36-483 of SEQ ID NO:1;

(iii) consists of an amino acid sequence selected from SEQ ID NOs: 60 to 115; or

(iv) has an amino acid sequence of SEQ ID NO: 99.

61. The method according to any one of paragraphs 43-60, wherein the hyaluronidase is present in the composition in an amount of from 100 U to 50,000 U.
62. The method according to any one of paragraphs 43-61, wherein the hyaluronidase is present in the composition in an amount of from 100 U/mL to 50,000 U/mL.
63. The method according to any one of paragraphs 43-62, wherein the composition further comprises one or more pharmaceutically acceptable excipients.
64. The method according to any one of paragraphs 43-63, wherein the composition further comprises one or more saccharides.
65. The method according to paragraph 64, wherein the one or more saccharides comprises a monosaccharide.
66. The method according to any one of paragraphs 64-65, wherein the one or more saccharides comprises a polysaccharide.
67. The method according to paragraph 66, wherein the one or more polysaccharides is selected from the group consisting of trehalose and sucrose.
68. The method according to any one of paragraphs 64-67, wherein the one or more saccharides is present in the composition in an amount from 10 mM to 500 mM, optionally in an amount from 100 mM to 300 mM.
69. The method according to any one of paragraphs 43-68, wherein the composition further comprises one or more amino acids.
70. The method according to paragraph 69, wherein the amino acids are selected from methionine and histidine.
71. The method according to any one of paragraphs 69-70, wherein the one or more amino acids is present in the composition in an amount from 1 mM to 100 mM, optionally in an amount from 1 mM to 50 mM.
72. The method according to any one of paragraphs 43-71 wherein the composition further comprises a buffer.
73. The method according to paragraph 72, wherein the buffer is present in the composition in an amount sufficient to maintain the composition at a pH from 3.0 to 9.0, optionally in an amount sufficient to maintain the composition at a pH from 5.5 to 7.5.
74. The method according to any one of paragraphs 72-73, wherein the buffer is present in the composition in an amount of from 1 to 100 mM, optionally in an amount of from 1 mM to 50 mM.
75. The method according to any one of paragraphs 72-74, wherein the buffer comprises histidine.
76. The method according to any one of paragraphs 43-75, wherein the oligonucleotide of the telomerase inhibitor comprises at least one N3′ 4 P5′ thiophosphoramidate internucleoside linkage.
77. The method according to any one of paragraphs 43-76, wherein the lipid moiety of the telomerase inhibitor is linked to the 5′ and/or 3′ end of the oligonucleotide via a linker.
78. The method according to paragraph 77, wherein the linker is a glycerol or aminoglycerol linker.
79. The method according to any one of paragraphs 43-78, wherein the lipid moiety of the telomerase inhibitor is a palmitoyl (C16) moiety.
80. The method according to any one of paragraphs 43-79, and 158, wherein the telomerase inhibitor is imetelstat or a pharmaceutically acceptable salt thereof, optionally imetelstat sodium.
81. A unit dosage form comprising a hyaluronidase enzyme and a telomerase inhibitor comprising an oligonucleotide and a lipid moiety linked to the 5′ and/or 3′ end of the oligonucleotide.
82. The unit dosage form according to paragraph 81, wherein the hyaluronidase enzyme is a recombinant human hyaluronidase.
83. The unit dosage form according to paragraph 82, wherein the hyaluronidase enzyme is rHuPH20.
84. The unit dosage form according to paragraph 83, wherein the composition comprises a variant or fragment of a PH20 hyaluronidase enzyme.
85. The unit dosage form according to paragraph 84, wherein the variant or fragment of PH20 comprises

(i) one or more amino acid residue substitutions selected from the group consisting of T341A, T341C, T341G, S343E, M345T, K349E, L353A, L354I, N356E and I361T in wild-type PH20 having the amino acid sequence of SEQ ID NO: 1;

(ii) one or more amino acid residue substitutions selected from the group consisting of T341A, T341C, L354I and N356E; or

(iii) one or more amino acid residue substitutions in the region corresponding to an alpha-helix region or a linker region in wild-type PH20 having the amino acid sequence of SEQ ID NO: 1.

86. The unit dosage form according to paragraph 85, wherein the alpha-helix region is an alpha-helix 8 region comprising the amino acid residues S347 to C381 and the linker region is a linker region comprising the amino acid residues A333 to R346 between alpha-helix 7 and alpha-helix 8.
87. The unit dosage form according to paragraph 85, wherein the alpha-helix region and the linker region comprises amino acid residues T341 to N363, T341 to I361, L342 to I361, S343 to I361, I344 to I361, M345 to I361, or M345 to N363.
88. The unit dosage form according to paragraph 86, wherein the alpha-helix 8 region and the linker region between alpha-helix 7 and alpha-helix 8 are substituted with one or more amino acid residues of the corresponding region of Hyal1.
89. The unit dosage form according to paragraph 84, wherein the variant or fragment of PH20 comprises

(i) one or more amino acid residue substitutions at one or more positions selected from the group consisting of T341, L342, S343, I344, M345, S347, M348, K349, L352, L353, D355, E359, I361 and N363;

(ii) amino acid residue substitutions of:

one or more of L354I and N356E; and

one or more amino acid residue substitutions selected from the group consisting of T341A, T341C, T341D, T341G, T341S, L342W, S343E, I344N, M345T, S347T, M348K, K349E, L352Q, L353A, D355K, E359D, I361T and N363G, optionally wherein the variant or fragment of PH20 comprises the substitutions M345T, S347T, M348K, K349E, L352Q, L353A, L354I, D355K, N356E, E359D and I361T;

(iii) the substitutions T341A, T341C, T341D, T341G, T341S, L342W, S343E, I344N and N363G; or

(iv) any one amino acid residue substitution selected from the following amino acid residue substitution groups:

(a) T341S, L342W, S343E, I344N, M345T, S347T, M348K, K349E, L352Q, L353A, L354I, D355K, N356E, E359D and I361T;

(b) L342W, S343E, I344N, M345T, S347T, M348K, K349E, L352Q, L353A, L354I, D355K, N356E, E359D and I361T;

(c) M345T, S347T, M348K, K349E, L352Q, L353A, L354I, D355K, N356E, E359D, I361T and N363G;

(d) T341G, L342W, S343E, I344N, M345T, S347T, M348K, K349E, L352Q, L353A, L354I, D355K, N356E, E359D and I361T;

(e) T341A, L342W, S343E, I344N, M345T, S347T, M348K, K349E, L352Q, L353A, L354I, D355K, N356E, E359D and I361T;

(f) T341C, L342W, S343E, I344N, M345T, S347T, M348K, K349E, L352Q, L353A, L354I, D355K, N356E, E359D and I361T;

(g) T341D, L342W, S343E, I344N, M345T, S347T, M348K, K349E, L352Q, L353A, L354I, D355K, N356E, E359D and I361T;

(h) I344N, M345T, S347T, M348K, K349E, L352Q, L353A, L354I, D355K, N356E, E359D and I361T; and

(i) S343E, I344N, M345T, S347T, M348K, K349E, L352Q, L353A, L354I, D355K, N356E, E359D and I361T.

90. The unit dosage form according to any one of paragraphs 84-89, wherein one or more of the N-terminal or C-terminal amino acid residues of the variant or fragment of PH20 are deleted.
91. The unit dosage form according to paragraph 90 wherein cleavage is positioned

(i) before an amino acid residue selected from the group consisting of M1 to P42 at the N-terminus such that one or more residues at the N-terminus are deleted;

(ii) before an amino acid residue L36, N37, F38, R39, A40, P41, or P42 at the N-terminus such that one or more residues at the N-terminus are deleted;

(iii) after an amino acid residue selected from the group consisting of V455 to L509 at the C-terminus such that one or more amino acid residues at the C-terminus are deleted;

(iv) after an amino acid residue selected from V455, C458, D461, C464, I465, D466, A467, F468, K470, P471, P472, M473, E474, T475, E476, E477, P478, Q479, I480, F481, Y482, N483, A484, P486, T488, or S490 at the C-terminus such that one or more amino acid residues at the C-terminus are deleted.

92. The unit dosage form according to any one of paragraphs 84-91, wherein the N-terminus comprises a human growth hormone-derived signal peptide having an amino acid sequence MATGSRTSLLLAFGLLCLPWLQEGSA of SEQ ID NO: 3, a human serum albumin-derived signal peptide having an amino acid sequence MKWVTFISLLFLFSSAYS of SEQ ID NO: 4, or a human Hyal1-derived signal peptide having an amino acid sequence

| of SEQ ID NO: 5 | |
|---|---|
| | MAAHLLPICALFLTLLDMAQG. |

93. The unit dosage form according to any one of paragraphs 84-92, wherein the variant or fragment of PH20

(i) is a peptide having at least 90% sequence identity to a sequence of amino acids set forth as SEQ ID NO:1 or amino acid residues 36-482, 36-477, 366-478, 36-479, 36-480, 36-481, and 36-483 of SEQ ID NO:1;

(ii) is a peptide having at least 95% sequence identity to a sequence of amino acids set forth as SEQ ID NO:1 or amino acid residues 36-482, 36-477, 366-478, 36-479, 36-480, 36-481, and 36-483 of SEQ ID NO:1;

(iii) consists of an amino acid sequence selected from SEQ ID NOs: 60 to 115; or

(iv) has an amino acid sequence of SEQ ID NO: 99.

94. The unit dosage form according to any one of paragraphs 81-93, wherein the hyaluronidase is present in the composition in an amount of from 100 U to 50,000 U.
95. The unit dosage form according to any one of paragraphs 81-94, wherein the hyaluronidase is present in the composition in an amount of from 100 U/mL to 50,000 U/mL.
96. The unit dosage form according to any one of paragraphs 81-95, wherein the composition further comprises one or more pharmaceutically acceptable excipients.
97. The unit dosage form according to any one of paragraphs 81-96, wherein the composition further comprises one or more saccharides.
98. The unit dosage form according to paragraph 97, wherein the one or more saccharides comprises a monosaccharide.
99. The unit dosage form according to any one of paragraphs 97-98, wherein the one or more saccharides comprises a polysaccharide.
100. The unit dosage form according to paragraph 99, wherein the one or more polysaccharides is selected from the group consisting of trehalose and sucrose.
101. The unit dosage form according to any one of paragraphs 97-100, wherein the one or more saccharides is present in the composition in an amount from 10 mM to 500 mM.
102. The unit dosage form according to any one of paragraphs 81-100, wherein the composition further comprises one or more amino acids.
103. The unit dosage form according to paragraph 102, wherein the amino acids are selected from methionine and histidine.
104. The unit dosage form according to any one of paragraphs 102-103, wherein the one or more amino acids is present in the composition in an amount from 1 mM to 100 mM, optionally in an amount from 1 mM to 50 mM.
105. The unit dosage form according to any one of paragraphs 81-104, wherein the composition further comprises a buffer.
106. The unit dosage form according to paragraph 105, wherein the buffer is present in the composition in an amount sufficient to maintain the composition at a pH from 3.0 to 9.0, optionally in an amount sufficient to maintain the composition at a pH from 5.5 to 7.5.
107. The unit dosage form according to any one of paragraphs 105-106, wherein the buffer is present in the composition in an amount of from 1 to 100 mM, optionally in an amount of from 1 mM to 50 mM.
108. The unit dosage form according to any one of paragraphs 81-107, wherein the oligonucleotide of the telomerase inhibitor comprises at least one N3′ 4 P5′ thiophosphoramidate internucleoside linkage.
109. The unit dosage form according to any one of paragraphs 81-108, wherein the lipid moiety of the telomerase inhibitor is linked to the 5′ and/or 3′ end of the oligonucleotide via a linker.
110. The unit dosage form according to paragraph 109, wherein the linker is a glycerol or aminoglycerol linker.
111. The unit dosage form according to any one of paragraphs 81-110, wherein the lipid moiety of the telomerase inhibitor is a palmitoyl (C16) moiety.
112. The unit dosage form according to any one of paragraphs 81-111, wherein the telomerase inhibitor is imetelstat or a pharmaceutically acceptable salt thereof, optionally imetelstat sodium.
113. The unit dosage form according to any one of paragraphs 81-112, wherein the telomerase inhibitor is present in the composition at a dosage of from

(i) about 2.0 mg/kg to 20.0 mg/kg;

(ii) about 3 mg/kg to about 15 mg/kg;

(iii) about 9 mg/kg to about 11 mg/kg; or

(iv) about 11 mg/kg to about 14 mg/kg.

114. The unit dosage form according to paragraphs 81-113, wherein the composition is liquid.
115. A kit comprising:

a composition comprising a hyaluronidase enzyme, and

a composition comprising a telomerase inhibitor comprising an oligonucleotide and a lipid moiety linked to the 5′ and/or 3′ end of the oligonucleotide.

116. The kit according to paragraph 115, further comprises an injector.
117. The kit according to paragraph 115-116, wherein the composition comprising a telomerase inhibitor is lyophilized.
118. The kit according to paragraph 115-117, wherein the kit further comprises a buffer for generating a reconstituted liquid composition.
119. The kit according to any one of paragraphs 115-118, wherein the subcutaneous injector comprises a needle and syringe.
120. The kit according to any one of paragraphs 115-119, wherein the subcutaneous injector is a bolus injector configured to subcutaneously deliver a predetermined amount of the composition.
121. The kit according to any one of paragraphs 115-120, wherein the hyaluronidase enzyme is a recombinant human hyaluronidase.
122. The kit according to paragraph 121, wherein the hyaluronidase enzyme is rHuPH20.
123. The kit according to any one of paragraphs 115-121, wherein the composition comprises a variant or fragment of a PH20 hyaluronidase enzyme.
124. The kit according to paragraph 123, wherein the variant or fragment of PH20 comprises

(i) one or more amino acid residue substitutions selected from the group consisting of T341A, T341C, T341G, S343E, M345T, K349E, L353A, L354I, N356E and I361T in wild-type PH20 having the amino acid sequence of SEQ ID NO: 1;

(ii) one or more amino acid residue substitutions selected from the group consisting of T341A, T341C, L354I and N356E;

(iii) one or more amino acid residue substitutions in the region corresponding to an alpha-helix region or a linker region in wild-type PH20 having the amino acid sequence of SEQ ID NO: 1.

125. The kit according to paragraph 124, wherein the alpha-helix region is an alpha-helix 8 region comprising the amino acid residues S347 to C381 and the linker region is a linker region comprising the amino acid residues A333 to R346 between alpha-helix 7 and alpha-helix 8.
126. The kit according to paragraph 124, wherein the alpha-helix region and the linker region comprises amino acid residues T341 to N363, T341 to I361, L342 to I361, S343 to I361, I344 to I361, M345 to I361, or M345 to N363.
127. The kit according to paragraph 124, wherein the alpha-helix 8 region and the linker region between alpha-helix 7 and alpha-helix 8 are substituted with one or more amino acid residues of the corresponding region of Hyal1.
128. The kit according to paragraph 123, wherein the variant or fragment of PH20 comprises one or more amino acid residue substitutions at one or more positions selected from the group consisting of T341, L342, S343, I344, M345, S347, M348, K349, L352, L353, D355, E359, I361 and N363.
129. The kit according to paragraph 128, wherein the variant or fragment of PH20 comprises

(i) amino acid residue substitutions of:

one or more of L354I and N356E; and

one or more amino acid residue substitutions selected from the group consisting of T341A, T341C, T341D, T341G, T341S, L342W, S343E, I344N, M345T, S347T, M348K, K349E, L352Q, L353A, D355K, E359D, I361T and N363G;

(ii) the substitutions M345T, S347T, M348K, K349E, L352Q, L353A, L354I, D355K, N356E, E359D and I361T;

(iii) the substitutions T341A, T341C, T341D, T341G, T341S, L342W, S343E, I344N and N363G; or

(iv) any one amino acid residue substitution selected from the following amino acid residue substitution groups:

(a) T341S, L342W, S343E, I344N, M345T, S347T, M348K, K349E, L352Q, L353A, L354I, D355K, N356E, E359D and I361T;

(b) L342W, S343E, I344N, M345T, S347T, M348K, K349E, L352Q, L353A, L354I, D355K, N356E, E359D and I361T;

(c) M345T, S347T, M348K, K349E, L352Q, L353A, L354I, D355K, N356E, E359D, I361T and N363G;

(d) T341G, L342W, S343E, I344N, M345T, S347T, M348K, K349E, L352Q, L353A, L354I, D355K, N356E, E359D and I361T;

(e) T341A, L342W, S343E, I344N, M345T, S347T, M348K, K349E, L352Q, L353A, L354I, D355K, N356E, E359D and I361T;

(f) T341C, L342W, S343E, I344N, M345T, S347T, M348K, K349E, L352Q, L353A, L354I, D355K, N356E, E359D and I361T;

(g) T341D, L342W, S343E, I344N, M345T, S347T, M348K, K349E, L352Q, L353A, L354I, D355K, N356E, E359D and I361T;

(h) I344N, M345T, S347T, M348K, K349E, L352Q, L353A, L354I, D355K, N356E, E359D and I361T; and

(i) S343E, I344N, M345T, S347T, M348K, K349E, L352Q, L353A, L354I, D355K, N356E, E359D and I361T.

130. The kit according to any one of paragraphs 123-129, wherein one or more of the N-terminal or C-terminal amino acid residues of the variant or fragment of PH20 are deleted.
131. The kit according to paragraph 130, wherein cleavage is positioned before an amino acid residue selected from the group consisting of M1 to P42 at the N-terminus such that one or more residues at the N-terminus are deleted.
132. The kit according to paragraph 131, wherein the cleavage is positioned

(i) before an amino acid residue L36, N37, F38, R39, A40, P41, or P42 at the N-terminus such that one or more residues at the N-terminus are deleted;

(ii) after an amino acid residue selected from the group consisting of V455 to L509 at the C-terminus such that one or more amino acid residues at the C-terminus are deleted; or

(iii) after an amino acid residue selected from V455, C458, D461, C464, I465, D466, A467, F468, K470, P471, P472, M473, E474, T475, E476, E477, P478, Q479, I480, F481, Y482, N483, A484, P486, T488, or S490 at the C-terminus such that one or more amino acid residues at the C-terminus are deleted.

133. The kit according to any one of paragraphs 123-132, wherein the N-terminus comprises a human growth hormone-derived signal peptide having an amino acid sequence MATGSRTSLLLAFGLLCLPWLQEGSA of SEQ ID NO: 3, a human serum albumin-derived signal peptide having an amino acid sequence MKWVTFISLLFLFSSAYS of SEQ ID NO: 4, or a human Hyal1-derived signal peptide having an amino acid sequence

| of SEQ ID NO: 5 | |
|---|---|
| | MAAHLLPICALFLTLLDMAQG. |

134. The kit according to any one of paragraphs 123-133, wherein the variant or fragment of PH20

(i) is a peptide having at least 90% sequence identity to a sequence of amino acids set forth as SEQ ID NO:1 or amino acid residues 36-482, 36-477, 366-478, 36-479, 36-480, 36-481, and 36-483 of SEQ ID NO:1;

(ii) is a peptide having at least 95% sequence identity to a sequence of amino acids set forth as SEQ ID NO:1 or amino acid residues 36-482, 36-477, 366-478, 36-479, 36-480, 36-481, and 36-483 of SEQ ID NO:1;

(iii) consists of an amino acid sequence selected from SEQ ID NOs: 60 to 115; or

(iv) has an amino acid sequence of SEQ ID NO: 99.

135. The kit according to any one of paragraphs 115-134, wherein the hyaluronidase is present in the composition in an amount of from 100 U to 50,000 U.
136. The kit according to any one of paragraphs 115-135, wherein the hyaluronidase is present in the composition in an amount of from 100 U/mL to 50,000 U/mL.
137. The kit according to any one of paragraphs 115-136, wherein the composition further comprises one or more pharmaceutically acceptable excipients.
138. The kit according to any one of paragraphs 115-137, wherein the composition further comprises one or more saccharides.
139. The kit according to paragraph 138 wherein the one or more saccharides comprises a monosaccharide.
140. The kit according to any one of paragraphs 138-139, wherein the one or more saccharides comprises a polysaccharide.
141. The kit according to paragraph 140, wherein the one or more polysaccharides is selected from the group consisting of trehalose and sucrose.
142. The kit according to any one of paragraphs 140-141, wherein the one or more polysaccharides is present in the composition in an amount from 10 mM to 500 mM, optionally in an amount from 100 mM to 300 mM.
143. The kit according to any one of paragraphs 115-142, wherein the composition further comprises one or more amino acids.
144. The kit according to paragraph 143, wherein the composition comprises methionine.
145. The kit according to any one of paragraphs 143-144, wherein the one or more amino acids is present in the composition in an amount from 1 mM to 100 mM, optionally in an amount from 1 mM to 50 mM.
146. The kit according to any one of paragraphs 115-145, wherein the composition further comprises a buffer.
147. The kit according to paragraph 146, wherein the buffer is present in the composition in an amount sufficient to maintain the composition at a pH from 3.0 to 9.0, optionally in an amount sufficient to maintain the composition at a pH from 5.5 to 7.5.
148. The kit according to any one of paragraphs 146-147, wherein the buffer is present in the composition in an amount of from 1 to 100 mM, optionally in an amount of from 1 mM to 50 mM.
149. The kit according to any one of paragraphs 146-148, wherein the buffer comprises histidine.
150. The kit according to any one of paragraphs 115-149, wherein the oligonucleotide of the telomerase inhibitor comprises at least one N3′ 4 P5′ thiophosphoramidate internucleoside linkage.
151. The kit according to any one of paragraphs 115-150, wherein the lipid moiety of the telomerase inhibitor is linked to the 5′ and/or 3′ end of the oligonucleotide via a linker.
152. The kit according to paragraph 151, wherein the linker is a glycerol or aminoglycerol linker.
153. The kit according to any one of paragraphs 115-152, wherein the lipid moiety of the telomerase inhibitor is a palmitoyl (C16) moiety.
154. The kit according to any one of paragraphs 115-153, wherein the telomerase inhibitor is imetelstat or a pharmaceutically acceptable salt thereof, optionally imetelstat sodium.
155. The kit according to any one of paragraphs 115-154, wherein the telomerase inhibitor is present in the composition at a dosage of from

(i) about 2.0 mg/kg to 20.0 mg/kg;

(ii) about 3 mg/kg to about 15 mg/kg;

(iii) about 9 mg/kg to about 11 mg/kg; or

(iv) about 11 mg/kg to about 14 mg/kg.

156. The kit according to any one of paragraphs 115-154, wherein the telomerase inhibitor is present in the composition at a dosage

(i) of from about 200 mg to 3000 mg;

(ii) of from about 750 mg to about 2500 mg;

(iii) of from about 1000 mg to about 2000 mg; or

(iv) of from about 500 mg to about 2000 mg.

157. The composition according to any one of paragraphs 1-40, wherein the telomerase inhibitor is present in the composition at a dosage

(i) of from about 200 mg to 3000 mg;

(ii) of from about 750 mg to about 2500 mg;

(iii) of from about 1000 mg to about 2000 mg; or

(iv) of from about 500 mg to about 2000 mg.

158. The method according to any one of paragraphs 43-47, wherein the telomerase inhibitor is present in the composition at a dosage

(i) of from about 200 mg to 3000 mg;

(ii) of from about 750 mg to about 2500 mg;

(iii) of from about 1000 mg to about 2000 mg; or

(iv) of from about 500 mg to about 2000 mg.

159. The method according to any one of paragraphs 81-112, wherein the telomerase inhibitor is present in the composition at a dosage

(i) of from about 200 mg to 3000 mg;

(ii) of from about 750 mg to about 2500 mg;

(iii) of from about 1000 mg to about 2000 mg; or

(iv) of from about 500 mg to about 2000 mg.

1. A composition formulated for subcutaneous administration, the composition comprising:
a telomerase inhibitor comprising an oligonucleotide and a lipid moiety linked to the 5′ and/or 3′ end of the oligonucleotide; and
a hyaluronidase enzyme.

2. The composition according to claim 1, wherein the hyaluronidase enzyme is a recombinant human hyaluronidase.

3. The composition according to claim 1, wherein the composition comprises a variant or fragment of a PH20 hyaluronidase enzyme.

4. The composition according to claim 3, wherein one or more of the N-terminal or C-terminal amino acid residues of the variant or fragment of PH20 are deleted.

5. The composition according to claim 4, wherein cleavage is positioned before an amino acid residue selected from the group consisting of M1 to P42 at the N-terminus such that one or more residues at the N-terminus are deleted.

6. The composition according to claim 5, wherein the cleavage is positioned before an amino acid residue L36, N37, F38, R39, A40, P41, or P42 at the N-terminus such that one or more residues at the N-terminus are deleted.

7. The composition according to claim 6, wherein the cleavage is positioned after an amino acid residue selected from the group consisting of V455 to L509 at the C-terminus such that one or more amino acid residues at the C-terminus are deleted.

8. The composition according to claim 7, wherein the cleavage is positioned after an amino acid residue selected from V455, C458, D461, C464, I465, D466, A467, F468, K470, P471, P472, M473, E474, T475, E476, E477, P478, Q479, I480, F481, Y482, N483, A484, P486, T488, or S490 at the C-terminus such that one or more amino acid residues at the C-terminus are deleted.

9. The composition according to claim 4, wherein the variant or fragment of PH20 comprises a polypeptide selected from the group set forth as amino acid residues 36-482, 36-477, 366-478, 36-479, 36-480, 36-481, and 36-483 of SEQ ID NO: 1.

10. The composition according to claim 4, wherein the N-terminus comprises a human growth hormone-derived signal peptide having an amino acid sequence MATGSRTSLLLAFGLLCLPWLQEGSA of SEQ ID NO: 3, a human serum albumin-derived signal peptide having an amino acid sequence MKWVTFISLLFLFSSAYS of SEQ ID NO: 4, or a human Hyal1-derived signal peptide having an amino acid sequence MAAHLLPICALFLTLLDMAQG of SEQ ID NO: 5.

11. The composition according to claim 2, wherein the hyaluronidase enzyme is rHuPH20.

12. The composition according to claim 4, wherein the variant or fragment of PH20 is a peptide having at least 90% sequence identity to a sequence of amino acids set forth as SEQ ID NO:1 or amino acid residues 36-482, 36-477, 366-478, 36-479, 36-480, 36-481, and 36-483 of SEQ ID NO:1.

13. The composition according to claim 4, wherein the variant or fragment of PH20 is a peptide having at least 95% sequence identity to a sequence of amino acids set forth as SEQ ID NO:1 or amino acid residues 36-482, 36-477, 366-478, 36-479, 36-480, 36-481, and 36-483 of SEQ ID NO:1.

14. The composition according to claim 1, wherein the hyaluronidase is present in the composition in an amount of from 100 U to 50,000 U.

15. The composition according to claim 1, wherein the composition further comprises one or more pharmaceutically acceptable excipients.

16. The composition according to claim 1, wherein the composition further comprises one or more saccharides.

17. The composition according to claim 16, wherein the one or more saccharides is present in the composition in an amount from 10 mM to 500 mM.

18. The composition according to claim 1, wherein the composition further comprises one or more amino acids.

19. The composition according to claim 18, wherein the amino acids are selected from methionine and histidine.

20. The composition according to claim 18, wherein the one or more amino acids is present in the composition in an amount from 1 mM to 100 mM.

21. The composition according to claim 1, wherein the composition further comprises a buffer.

22. The composition according to claim 21, wherein the buffer is present in the composition in an amount sufficient to maintain the composition at a pH from 3.0 to 9.0.

23. The composition according to claim 21, wherein the buffer is present in the composition in an amount of from 1 to 100 mM.

24. The composition according to claim 1, wherein the oligonucleotide of the telomerase inhibitor comprises at least one N3′ 4 P5′ thiophosphoramidate internucleoside linkage.

25. The composition according to claim 1, wherein the lipid moiety of the telomerase inhibitor is linked to the 5′ and/or 3′ end of the oligonucleotide via a linker.

26. The composition according to claim 25, wherein the linker is a glycerol or aminoglycerol linker.

27. The composition according to claim 1, wherein the lipid moiety of the telomerase inhibitor is a palmitoyl (C16) moiety.

28. The composition according to claim 1, wherein the telomerase inhibitor is imetelstat or a pharmaceutically acceptable salt thereof.

29. The composition according to claim 28 wherein the telomerase inhibitor is imetelstat sodium.

30. The composition according to claim 1, wherein the telomerase inhibitor is present in the composition at a dosage of from
(i) about 2.0 mg/kg to 20.0 mg/kg;
(ii) about 3 mg/kg to about 15 mg/kg;
(iii) about 9 mg/kg to about 11 mg/kg; or
(iv) about 11 mg/kg to about 14 mg/kg.

31. The composition according to claim 1, wherein the telomerase inhibitor is present in the composition at a dosage
(i) of from about 200 mg to 3000 mg;
(ii) of from about 750 mg to about 2500 mg;
(iii) of from about 1000 mg to about 2000 mg; or
(iv) of from about 500 mg to about 2000 mg.

32. The composition according to claim 1, wherein the composition is lyophilized.

33. A method of treating a subject having a neoplasm, the method comprising subcutaneously administering to the subject a composition comprising:
a telomerase inhibitor comprising an oligonucleotide and a lipid moiety linked to the 5′ and/or 3′ end of the oligonucleotide; and
a hyaluronidase enzyme.

34.-69. (canceled)

70. A unit dosage form comprising a hyaluronidase enzyme and a telomerase inhibitor comprising an oligonucleotide and a lipid moiety linked to the 5′ and/or 3′ end of the oligonucleotide.

71.-101. (canceled)

102. A kit comprising:
a composition comprising a hyaluronidase enzyme, and
a composition comprising a telomerase inhibitor comprising an oligonucleotide and a lipid moiety linked to the 5′ and/or 3′ end of the oligonucleotide.

103.-136. (canceled)


SEQUENCE LISTING (83 sequences)

SEQ ID NO: 1 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 2 - protein, 435 aa, Homo sapiens
MAAHLLPICALFLTLLDMAQGFRGPLLPNRPFTTVWNANTQWCLERHGVDVDVSVFDVVANPGQTFRGPDMTIFYSSQLGTYPYYTPTGEPVFGGLPQNASLIAHLARTFQDILAAPIPAPDFSGLAVIDWEAWRPRWAFNWDTKDIYRQRSRALVQAQHPDWPAPQVEAVAQDQFQGAARAWMAGTLQLGRALRPRGLWGFYGFPDCYNYDFLSPNYTGQCPSGIRAQNDQLGWLWGQSRALYPSIYMPAVLEGTGKSQMYVQHRVAEAFRVAVAAGDPNLPVLPYVQIFYDTTNHFLPLDELEHSLGEAAQGAAGVVLWVSWENTRTKESCQAIKEYMDTTLGPFILNVTSGALLCSQALCSGHGRCVRRTSHPKALLLLNPASFSIQLTPGGGPLSLRGALSLEDQAQMAVEFKCRCYPGWQAPWCERKSMW

SEQ ID NO: 3 - protein, 26 aa, Homo sapiens
MATGSRTSLLLAFGLLCLPWLQEGSA

SEQ ID NO: 4 - protein, 18 aa, Homo sapiens
MKWVTFISLLFLFSSAYS

SEQ ID NO: 5 - protein, 21 aa, Homo sapiens
MAAHLLPICALFLTLLDMAQG

SEQ ID NO: 6 - RNA, 554 nt, Artificial Sequence
  Synthetic Sequence
ggguugcggagggugggccugggaggggugguggccauuuuuugucuaacccuaacugagaagggcguaggcgccgugcuuuugcuccccgcgcgcuguuuuucucgcugacuuucagcgggcggaaaagccucggccugccgccuuccaccguucauucuagagcaaacaaaaaaugucagcugcuggcccguucgccucccggggaccugcggcgggucgccugcccagcccccgaaccccgccuggagccgcggucggcccggggcuucuccggaggcacccacugccaccgcgaagaguugggcucugucagccgcgggucucucgggggcgagggcgagguucaccguuucaggccgcaggaagaggaacggagcgagucccgccgcggcgcgauucccugagcugugggacgugcacccaggacucggcucacacaugcaguucgcuuuccuguugguggggggaacgccgaucgugcgcauccgucaccccucgccggcagugggggcuugugaacccccaaaccugacugacugggccagugugcu

SEQ ID NO: 7 - DNA, 20 bp, Artificial Sequence
  Synthetic Sequence
acattttttgtttgctctag

SEQ ID NO: 8 - DNA, 30 bp, Artificial Sequence
  Synthetic Sequence
gctctagaatgaacggtggaaggcggcagg

SEQ ID NO: 9 - DNA, 14 bp, Artificial Sequence
  Synthetic Sequence
gtggaggcggcagg

SEQ ID NO: 10 - DNA, 13 bp, Artificial Sequence
  Synthetic Sequence
ggaaggcggcagg

SEQ ID NO: 11 - DNA, 13 bp, Artificial Sequence
  Synthetic Sequence
gtggaaggcggca

SEQ ID NO: 12 - DNA, 11 bp, Artificial Sequence
  Synthetic Sequence
gtggaaggcgg

SEQ ID NO: 13 - DNA, 13 bp, Artificial Sequence
  Synthetic Sequence
cggtggaaggcgg

SEQ ID NO: 14 - DNA, 13 bp, Artificial Sequence
  Synthetic Sequence
acggtggaaggcg

SEQ ID NO: 15 - DNA, 16 bp, Artificial Sequence
  Synthetic Sequence
aacggtggaaggcggc

SEQ ID NO: 16 - DNA, 18 bp, Artificial Sequence
  Synthetic Sequence
atgaacggtggaaggcgg

SEQ ID NO: 17 - DNA, 13 bp, Artificial Sequence
  Synthetic Sequence
tagggttagacaa

SEQ ID NO: 18 - DNA, 13 bp, Artificial Sequence
  Synthetic Sequence
cagttagggttag

SEQ ID NO: 19 - DNA, 12 bp, Artificial Sequence
  Synthetic Sequence
tagggttagaca

SEQ ID NO: 20 - DNA, 11 bp, Artificial Sequence
  Synthetic Sequence
tagggttagac

SEQ ID NO: 21 - DNA, 11 bp, Artificial Sequence
  Synthetic Sequence
gttagggttag

SEQ ID NO: 22 - DNA, 13 bp, Artificial Sequence
  Synthetic Sequence
gttagggttagac

SEQ ID NO: 23 - DNA, 15 bp, Artificial Sequence
  Synthetic Sequence
gttagggttagacaa

SEQ ID NO: 24 - DNA, 9 bp, Artificial Sequence
  Synthetic Sequence
gggttagac

SEQ ID NO: 25 - DNA, 9 bp, Artificial Sequence
  Synthetic Sequence
cagttaggg

SEQ ID NO: 26 - DNA, 12 bp, Artificial Sequence
  Synthetic Sequence
cccttctcagtt

SEQ ID NO: 27 - DNA, 12 bp, Artificial Sequence
  Synthetic Sequence
cgcccttctcag

SEQ ID NO: 28 - protein, 455 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLGPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 29 - protein, 454 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPILNVTSGALLCSQALLQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 30 - protein, 455 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLGPFILNVTSGALLCSQALCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 31 - protein, 454 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWVSWENTRTKESCQAIKEMDTTLGPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 32 - protein, 454 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITREKSCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 33 - protein, 455 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWVSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 34 - protein, 455 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSNTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 35 - protein, 455 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLENTRTKESCQAIKEYMDTTLNPYIINVTLAAKMCSQVLCQEQGYCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 36 - protein, 455 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRHDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 37 - protein, 455 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 38 - protein, 454 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTSGAKMCSQVLCQEQGCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 39 - protein, 453 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 40 - protein, 430 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCI

SEQ ID NO: 41 - protein, 433 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGYCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 42 - protein, 436 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKP

SEQ ID NO: 43 - protein, 451 aa, Artificial Sequence
  Synthetic Sequence
FRGPLLPNRPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 44 - protein, 453 aa, Artificial Sequence
  Synthetic Sequence
FRGPLLPNRPFTTVWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 45 - protein, 433 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLK

SEQ ID NO: 46 - protein, 430 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 47 - protein, 455 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 48 - protein, 451 aa, Artificial Sequence
  Synthetic Sequence
APPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 49 - protein, 449 aa, Artificial Sequence
  Synthetic Sequence
PVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 50 - protein, 432 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 51 - protein, 429 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVC

SEQ ID NO: 52 - protein, 425 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGCIRKNWNSSDYLHLNPDNFAIQLEKGGKFTVRGKPTLEDLEQFSEKFYCSCYSTLSCKEKADVKDTDAVDVCIAD

SEQ ID NO: 53 - protein, 423 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVC

SEQ ID NO: 54 - protein, 420 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAV

SEQ ID NO: 55 - protein, 433 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLK

SEQ ID NO: 56 - protein, 435 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPP

SEQ ID NO: 57 - protein, 436 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPM

SEQ ID NO: 58 - protein, 437 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPME

SEQ ID NO: 59 - protein, 438 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMET

SEQ ID NO: 60 - protein, 439 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETE

SEQ ID NO: 61 - protein, 454 aa, Artificial Sequence
  Synthetic Sequence
NFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 62 - protein, 452 aa, Artificial Sequence
  Synthetic Sequence
RAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 63 - protein, 450 aa, Artificial Sequence
  Synthetic Sequence
PPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 64 - protein, 428 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCI

SEQ ID NO: 65 - protein, 429 aa, Artificial Sequence
  Synthetic Sequence
TRAPPVIPNVPFLWAWNAPSEFCLGKFDEPLDMSLTSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCID

SEQ ID NO: 66 - protein, 430 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 67 - protein, 431 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 68 - protein, 441 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 69 - protein, 443 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 70 - protein, 445 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 71 - protein, 447 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNA

SEQ ID NO: 72 - protein, 449 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASP

SEQ ID NO: 73 - protein, 450 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPIFYNASPST

SEQ ID NO: 74 - protein, 455 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGGWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 75 - protein, 455 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGAWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 76 - protein, 455 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGCWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 77 - protein, 454 aa, Artificial Sequence
  Synthetic Sequence
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 78 - protein, 431 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSNTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 79 - protein, 431 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPVHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 80 - protein, 431 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGAWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 81 - protein, 429 aa, Artificial Sequence
  Synthetic Sequence
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGGWENTRESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 82 - protein, 429 aa, Artificial Sequence
  Synthetic Sequence
FRGPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 83 - protein, 430 aa, Artificial Sequence
  Synthetic Sequence
FRGPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF